SEQUENCE LISTING (41 sequences)

SEQ ID NO: 1 - DNA, 29903 bp, SARS CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - protein, 4405 aa, SARS CoV-2 [sequence omitted]

SEQ ID NO: 3 - protein, 932 aa, SARS CoV-2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 4 - protein, 601 aa, SARS CoV-2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 5 - protein, 1273 aa, SARS CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 254 aa, SARS CoV-2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 7 - protein, 610 aa, SARS CoV-2
CDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLH

SEQ ID NO: 8 - protein, 1945 aa, SARS CoV-2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 9 - protein, 500 aa, SARS CoV-2
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 10 - protein, 275 aa, SARS CoV-2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 11 - protein, 75 aa, SARS CoV-2
MYSFVSEETGTLIVNSVLLFLAFVVFLLVTLAILTALRLCAYCCNIVNVSLVKPSFYVYSRVKNLNSSRVPDLLV

SEQ ID NO: 12 - protein, 222 aa, SARS CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 13 - protein, 61 aa, SARS CoV-2
MFHLVDFQVTIAEILLIIMRTFKVSIWNLDYIINLIIKNLSKSLTENKYSQLDEEQPMEID

SEQ ID NO: 14 - protein, 121 aa, SARS CoV-2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 15 - protein, 43 aa, SARS CoV-2
MIELSLIDFYLCFLAFLLFLVLIMLIIFWFSLELQDHNETCHA

SEQ ID NO: 16 - protein, 121 aa, SARS CoV-2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 17 - protein, 419 aa, SARS CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 18 - skipped

SEQ ID NO: 19 - protein, 38 aa, SARS CoV-2
MGYINVFAFPFTIYSLLLCRMNSRNYIAQVDVVNFNLT

SEQ ID NO: 20 - DNA, 36 bp, SARS CoV-2
ttgtgcagaatgaattctcgtaactacatagcacaa

SEQ ID NO: 21 - DNA, 29 bp, SARS CoV-2
taactacatagcacaagtagatgtagtta

SEQ ID NO: 22 - protein, 113 aa, SARS CoV-2
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 23-24 - skipped (2 entries)

SEQ ID NO: 25 - RNA, 24 nt, Artificial Sequence
  A synthetic sequence
acgaaccuaaacacgaaccuaaac

SEQ ID NO: 26 - RNA, 24 nt, Artificial Sequence
  A synthetic sequence
acgaacacgaacacgaacacgaac

SEQ ID NO: 27 - RNA, 24 nt, Artificial Sequence
  A synthetic sequence
cuaaaccuaaaccuaaaccuaaac

SEQ ID NO: 28 - DNA, 450 bp, SARS CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcc

SEQ ID NO: 29 - DNA, 361 bp, SARS CoV-2
gaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 30 - DNA, 1590 bp, SARS CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccagaattagatctctcgaggttaacgaattctgctatacgaagttatccctc

SEQ ID NO: 31 - DNA, 713 bp, SARS CoV-2
atttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 32 - DNA, 450 bp, SARS CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcc

SEQ ID NO: 33 - DNA, 1590 bp, SARS CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccagaattagatctctcgaggttaacgaattctgctatacgaagttatccctc

SEQ ID NO: 34-35 - skipped (2 entries)

SEQ ID NO: 36 - RNA, 6 nt, SARS CoV-2
cuaaac

SEQ ID NO: 37 - RNA, 6 nt, SARS CoV-2
acgaac

SEQ ID NO: 38 - RNA, 10 nt, SARS CoV-2
cuaaacgaac

SEQ ID NO: 39 - RNA, 24 nt, Artificial Sequence
  A synthetic sequence
acgaaccuaaacacgaaccuaaac

SEQ ID NO: 40 - RNA, 24 nt, Artificial Sequence
  A synthetic sequence
acgaacacgaacacgaacacgaac

SEQ ID NO: 41 - RNA, 24 nt, Artificial Sequence
  A synthetic sequence
cuaaaccuaaaccuaaaccuaaac

(19) United States
(12) Patent Application Publication
(10) Pub. No.: US 2023/0151367 A1
(43) Pub. Date: May 18, 2023

(54) THERAPEUTIC INTERFERING PARTICLES FOR CORONA VIRUS

(71) Applicants: The J. David Gladstone Institutes, A Testamentary Trust Established Under the Will of J. David Glads, San Franciso, CA (US); VxBiosciences, Inc., San Francisco, CA (US)

(72) Inventors: Sonali CHATURVEDI, San Francisco, CA (US); Robert RODICK, Berkeley, CA (US); Leor S. WEINBERGER, San Francisco, CA (US)

(21) Appl. No.: 17/920,682

(22) PCT Filed: Apr. 23, 2021
(86) PCT No.: PCT/US2021/028809

§ 371 Date: Oct. 21, 2022

Related U.S. Application Data
(60) Provisional application No. 63/014,394, filed on Apr. 23, 2020.

(51) Int. Cl. C12N 15/113; C07K 14/005; C12N 15/10; C12N 15/86; C40B 40/02

(52) U.S. Cl. CPC C12N 15/1131; C07K 14/005; C12N 15/1058; C12N 15/1065; C12N 15/86; C40B 40/02; C12N 2310/11; C12N 2770/20021; C12N 2770/20022

(57) ABSTRACT

Described herein are compositions defective SARS-CoV-2 constructs and particles that can interfere with or block infection of uninfected cells and methods for generating such defective SARS-CoV-2 constructs and particles. The compositions and methods described herein are useful for treatment of SARS-CoV-2 infections.

### PRIORITY APPLICATIONS

This application claims the benefit of priority of U.S. Provisional Application Ser. No. 63/014,394, filed Apr. 23, 2020, the content of which is specifically incorporated herein by reference in its entirety.

### GOVERNMENT SUPPORT

This invention was made with government support under 1-DP2-OD006677-01 awarded by the National Institutes of Health and under D17AC00009 awarded by DOD/DARPA. The government has certain rights in the invention.

### INCORPORATION BY REFERENCE OF SEQUENCE LISTING PROVIDED BY AS A TEXT FILE

A Sequence Listing is provided herewith as a text file, “2135793.txt” created on Apr. 23, 2021 and having a size of 143,360 bytes. The contents of the text file are incorporated by reference herein in their entirety.

### BACKGROUND

The World Health Organization has declared Covid-19 a global pandemic. A highly infectious coronavirus, officially called SARS-CoV-2, causes the Covid-19 disease. Even with the most effective containment strategies, the spread of the Covid-19 respiratory disease has only been slowed. While effective vaccines exist for current strain of SARS-CoV-2, new variants and mutant strains continue to develop. Hence, there is a need for treatments that interfere with infection as well and/or new vaccines that can facilitate recovery from infection and put an end to the SARS-CoV-2 pandemic.

### SUMMARY

Provided are defective SARS-CoV-2 constructs and methods for generating defective SARS-CoV-2 constructs that can interfere with or block infection of uninfected cells. The methods and compositions are useful for treatment of SARS-CoV-2 infections.

The defective SARS-CoV-2 constructs described herein are SARS-CoV-2 recombinant deletion mutants. Such recombinant SARS-CoV-2 deletion mutants can be interfering and/or conditionally replicating SARS-CoV-2 deletion mutants. Even without non-SARS-CoV-2 nucleic acids the SARS-CoV-2 constructs can be therapeutic interfering particles or therapeutic interfering nucleic acids.

These constructs can include cis-acting elements comprising a 5′ untranslated region (5′ UTR), a 3′ untranslated region (3′ UTR), a poly-A tail, or a combination thereof; and SARS-CoV-2 genomic nucleic acid segments. Typically, the SARS-CoV-2 genomic nucleic acid segments have substantial deletions relative to the wild type SARS-CoV-2 genome. Hence, the therapeutic interfering SARS-CoV-2 nucleic acids and particles can be incapable of replication and production of virus on their own, and can, for example, require replication-competent SARS-CoV-2 to act as a helper virus.

Examples of such therapeutic interfering particles, defective SARS-CoV-2 constructs, and therapeutic interfering nucleic acids can include any of the 5′ SARS-CoV-2 truncated sequences such as any of those with SEQ ID NO:28, 30, 32 or 33 and/or any of the 3′ SARS-CoV-2 truncated sequences such as any of those with SEQ ID NO:31 or 32. The 3′ SARS-CoV-2 sequences can include extended poly A sequences. For example, the extended poly-A sequences can have at least 100 adenine nucleotides to 250 adenine nucleotides. Such extended poly-A sequences can, for example, extend the half-life of the mRNA.

The SARS-CoV-2 therapeutic interfering particles can therefore include an RNA transcription signals, translation initiation sites, extended poly-A tails, or a combination thereof. In addition, to the deletions, the SARS-CoV-2 genomic nucleic acid segments can have one or more nucleotide sequence alterations compared to a wild type or native SARS-CoV-2 genomic nucleotide sequence.

Also described herein are one or more inhibitors of transcription regulating sequences (TRSs): TRS1-L: 5′-cuaaac-3′ (SEQ ID NO:36), TRS2-L: 5′-acgaac-3′ (SEQ ID NO:37), and TRS3-L, 5′-cuaaacgaac-3′ (SEQ ID NO:38), and compositions thereof. The TRS inhibitors can be used alone or in conjunction with therapeutic interfering particles SARS-CoV-2 constructs to inhibit and/or interfere with SARS-CoV-2 infection.

The therapeutic interfering SARS-CoV-2 nucleic acids and/or the TRS inhibitors can, for example, block wild type SARS-CoV-2 cellular entry, compete for structural proteins that mediate viral particle assembly, reduce the reproduction of wild type SARS-CoV-2, produce proteins that inhibit assembly of viral particles, inhibit transcription/replication of SARS-CoV-2 nucleic acids, or a combination thereof.

Methods are also described herein that include making and using a SARS-CoV-2 deletion library. In some embodiments, a subject method includes: (a) inserting transposon cassette comprising a target sequence for a sequence specific DNA endonuclease into a population of circular SARS-CoV-2 DNAs to generate a population of transposon-inserted circular SARS-CoV-2 DNAs; (b) contacting the population of transposon-inserted circular SARS-CoV-2 DNAs with the sequence specific DNA endonuclease to generate a population of cleaved linear SARS-CoV-2 DNAs; (c) contacting the population of cleaved linear SARS-CoV-2 DNAs with one or more exonucleases to generate a population of SARS-CoV-2 deletion DNAs; and (d) circularizing the SARS-CoV-2 deletion DNAs to generate a library of circularized SARS-CoV-2 deletion DNAs.

In some cases, the transposon cassette includes a first recognition sequence positioned at or near one end of the transposon cassette and a second recognition sequence positioned at or near the other end of the transposon cassette.

In some such cases, the method further includes introducing members of the library of circularized SARS-CoV-2 deletion DNAs into mammalian cells and assaying for viral infectivity. For example, the SARS-CoV-2 deletion DNAs can be introduced to epithelial cells, or alveolar cells (e.g., human alveolar type II cells). In some cases, the method further includes sequencing members of the library of circularized SARS-CoV-2 deletion DNAs to identify defective SARS-CoV-2 interfering particles (DIPs).

In some cases, the sequence specific DNA endonuclease is selected from: a meganuclease, a CRISPR/Cas endonuclease, a zinc finger nuclease, or a TALEN. In some cases, the one or more exonucleases includes T4 DNA polymerase. In some cases, the one or more exonucleases includes a 3′ to 5′ exonuclease and a 5′ to 3′ exonuclease. In some cases, the one or more exonucleases includes RecJ. In some cases, a subject method includes inserting a barcode sequence prior to or simultaneous with step (d).

In some cases, the step of contacting the population of cleaved linear SARS-CoV-2 DNAs with one or more exonucleases is performed in the presence of a single strand binding protein (SSB).

Also provided are methods of generating and identifying a defective SARS-CoV-2 interfering particle (DIP). In some cases, the methods include (a) inserting a target sequence for a sequence specific DNA endonuclease into a population of circular SARS-CoV-2 viral DNAs, each comprising a viral genome, to generate a population of sequence-inserted SARS-CoV-2 viral DNAs; (b) contacting the population of sequence-inserted SARS-CoV-2 viral DNAs with the sequence specific DNA endonuclease to generate a population of cleaved linear SARS-CoV-2 viral DNAs; (c) contacting the population of cleaved linear SARS-CoV-2 viral DNAs with an exonuclease to generate a population of deletion DNAs; (d) circularizing the SARS-CoV-2 deletion DNAs to generate a library of circularized SARS-CoV-2 deletion viral DNAs; and (e) sequencing members of the library of circularized deletion SARS-CoV-2 viral DNAs to identify SARS-CoV-2 deletion interfering particles (DIPs). In some cases, the method includes inserting a barcode sequence prior to or simultaneous with step (d).

In some cases, the method includes introducing members of the generated library of circularized SARS-CoV-2 deletion DNAs into cells, for example, mammalian cells, and assaying for viral infectivity. In some cases, the inserting of step (a) includes inserting a transposon cassette into the population of circular SARS-CoV-2 viral DNAs, where the transposon cassette includes the target sequence for the sequence specific DNA endonuclease, and wherein said generated population of sequence-inserted SARS-CoV-2 viral DNAs is a population of transposon-inserted viral DNAs. In some cases, the method includes, after step (d), infecting cells, for example, mammalian cells in culture with members of the library of circularized deletion SARS-CoV-2 viral DNAs at a high multiplicity of infection (MOI), culturing the infected cells for a period of time ranging from 12 hours to 2 days, adding naive cells to the to the culture, and harvesting virus from the cells in culture. In some cases, the method includes, after step (d), infecting cells, for example, mammalian cells in culture with members of the library of circularized deletion viral DNAs at a low multiplicity of infection (MOI), culturing the infected cells in the presence of an inhibitor of viral replication for a period of time ranging from 1 day to 6 days, infecting the cultured cells with functional virus at a high MOI, culturing the infected cells for a period of time ranging from 12 hours to 4 days, and harvesting virus from the cultured cells.

### DESCRIPTION OF THE FIGURES

FIG. 1 shows a schematic diagram of the SARS-CoV-2 genome and encoded open reading frames (ORFs).

FIG. 2A-2B illustrate infection of cells by wild type and defective SARS-CoV-2. FIG. 2A shows a schematic representation of infection by a wildtype SARS-CoV-2 genome. After integration into a cellular genome (DNA at left), SARS-CoV-2 RNAs are generated that ultimately produce the packaging proteins that form the virus capsid. Infective SARS-CoV-2 can escape their original host cell and infect new cells if they have the needed (functional wild type) surface recognition proteins. FIG. 2B shows a schematic of infection when defective SARS-CoV-2 particles (referred to as Therapeutic Interfering Particles, TIPs) are present with viable SARS-CoV-2. The defective SARS-CoV-2 particles have pared-down versions of the SARS-CoV-2 genome engineered to carry a packaging signal, and other viral cis elements required for packaging. The defective SARS-CoV-2 RNA can thus only be made by cells that also express SARS-CoV-2 proteins. The defective SARS-CoV-2 particles are engineered to produce substantially more defective SARS-CoV-2 genomic RNA copies than wild type SARS-CoV-2 in dually infected cells. With disproportionately more defective SARS-CoV-2 genomic RNA than wild type SARS-CoV-2 genomic RNA, the SARS-CoV-2 packaging materials are mainly wasted enclosing defective SARS-CoV-2 genomic RNA. The defective SARS-CoV-2 particles lower the wild type SARS-CoV-2 burst size and convert infected cells from producing wild type SARS-CoV-2 into producing mostly defective SARS-CoV-2 particles, thereby lowering the wild type SARS-CoV-2 viral load.

FIG. 3 schematically illustrates a method for constructing a randomized, barcoded deletion library for making defective SARS-CoV-2 particles. The schematic cycle method for constructing a barcoded TIP candidate library from a molecular clone involves: [1] in vitro introduction of a retrotransposition into circular SARS-CoV-2 double stranded DNA, [2] exonuclease-mediated excision of the randomly inserted retrotransposon, [3] enzymatic chew back to create a deletion (A) in the circular SARS-CoV-2, and [4] circularizing and barcoding during re-ligation to generate the barcoded TIP candidate library (see, e.g., WO201811225 by Weinberger et al. and WO/2014/151771 by Weinberger et al., which are both incorporated herein by reference in their entireties).

FIG. 4 a schematic diagram illustrating molecular details and steps for one embodiment of a method of generating a deletion library. In step (a) the meganuclease (e.g., 1-Sce1 or 1-Ceu1) cleaves the SARS-CoV-2 double stranded DNA. In step (b) the cleaved ends of the SARS-CoV-2 DNA are chewed back. In step (c), the chewed back ends are repaired. Thus, a deleted gap (A) is present between the ends. In step (d) the 5′ phosphate is removed by alkaline phosphatase (AP) and a dA tail is generated with Klenow. In step (e), the ends are ligated to a barcode cassette, thereby generating numerous circular, barcoded deletion SARS-CoV-2 mutants.

FIG. 5A-5C illustrate methods for generating and analyzing random deletion libraries of SARS-CoV-2 deletion mutants. FIG. 5A schematically illustrates generation of a random deletion library (RDL) for a 30 kb SARS-CoV-2 molecular clone. Three 10 kb fragments are shown that were used for RDL sub-libraries, where the three fragments were different segments of the SARS-CoV-2 genome. The ends of the three fragments were chewed back (e.g., as described in FIG. 4), and the barcodes (shaded circles) were inserted as the deleted SARS-CoV-2 DNA fragments were ligated. Hence, the barcodes will be at different positions along the fragments. Because the barcodes include sites for primer initiation, sequencing readily identified where the deletions reside in the different SARS-CoV-2 deletion mutants. FIG. 5B graphically illustrates illumina deep sequencing landscapes of barcode positions in the three random deletion sub-libraries. Such sequencing showed that the sub-libraries contain more than 587,000 unique SARS-CoV-2 deletion mutants. FIG. 5C shows gels of electrophoretically separated DNA from the ligated RDL libraries illustrating that there are bands of about 30 kb as well as lower molecular weight bands (ladder is in left lane: the 3 additional lanes are triplicates).

FIG. 6A-6D illustrate the ‘viroreactor’ strategy used to generate SARS-CoV-2 therapeutic interfering particles (TIPs). FIG. 6A schematically illustrates VeroE6 cells that were immobilized on beads, grown in suspension under gentle agitation, and infected with SARS-CoV-2 at the indicated MOI. 50% of the cells and media were harvested and replaced every other day. FIG. 6B shows flow cytometry plots of harvested cells stained for Propidium Iodide, a cell death marker. FIG. 6C graphically illustrates the percentage cell viability following SARS-CoV-2 infection at a MOI of 0.5. FIG. 6D graphically illustrates the cell viability (%) following SARS-CoV-2 infection at a MOI of 5.0. As shown in FIG. 6C-6D, the percentage of viable free cells (circular symbols) and viable immobilized cells (triangular symbols) exhibit an initial dip in cell viability, but the cultures recover by day 14 post infection.

FIG. 7A-7B schematically illustrate the structures of two therapeutic interfering particles constructs for SARS-CoV-2, TIP1 and TIP2. FIG. 7A shows an example of the TIP1 construct structure. FIG. 7B shows an example of the TIP2 construct structure. The schematics show that TIP1 and TIP2 encode portions of the 5′ and 3′ untranslated regions (UTRs) of SARS-CoV-2. TIP1 encodes 450nt of 5′UTR and 330nt of 3′UTR. TIP2 includes the 5′UTR region and a larger portion of SARS-CoV-2 ORF1a (i.e., TIP2 encodes a deletion of ORF1a). Hence, TIP1 and TIP2 include the packaging signal but cannot express a functional copy of the viral ORF1a gene. The 3′UTR that is encoded by the TIP2 extends upstream 413nt into the SARS-COV-2 N gene but TIP2 does not encode a functional form of the N gene (i.e., it encodes a deletion of part of the N gene). To facilitate analysis, the cassettes also include an IRES-mCherry reporter for flow cytometry analysis.

FIG. 8A-8C graphically illustrate that four different types of therapeutic interfering particles (TIPs) reduce SARS-CoV-2 replication by more than 50-fold. FIG. 8A graphically illustrates the fold change in with SARS-CoV-2 RNA when various therapeutic interfering particles (TIPs) are present. Cells were transfected with mRNA of TIP1 (T1), TIP1* (T1*), TIP2 (T2) or TIP2* (T2*) and the cells were infected with SARS-CoV-2 (MOI=0.005). Yield-reduction of SARS-CoV-2 replication was assessed by measuring the fold-reduction in SARS-CoV-2 mRNA (E gene) at 48 hrs post infection. mRNA was quantified by RT-qPCR with primers specific to 5′-end of N gene and the E gene that are not present in TIPs. The fold-reduction in SARS-CoV-2 mRNA as detected by E gene primers is shown. TIP2 exhibits the greatest interference with SARS-CoV-2. FIG. 8B graphically illustrates the relative Log 10 amounts of SARS-CoV-2 genome when TIP1 and TIP2 therapeutic interfering particles are incubated for about 24 hours with the SARS-CoV-2 genome, as compared to control without the therapeutic interfering particles. FIG. 8C graphically illustrates the relative Log 10 amounts of SARS-CoV-2 genome when TIP1 and TIP2 therapeutic interfering particles are incubated for about 48 hours with the SARS-CoV-2 genome, as compared to control without the therapeutic interfering particles.

FIG. 9A-9B illustrate that TIP candidates are mobilized by SARS-CoV-2 and transmit together with SARS-CoV-2. FIG. 9A shows flow cytometry analysis of mCherry expression by Vero cells that received supernatant transferred from SARS-CoV-2 infected cells incubated with TIP1 and TIP2 therapeutic interfering particles compared to control cells receiving supernatant from naïve uninfected cells that were incubated with the TIP1 and TIP2 particles. As shown, mCherry-expressing cells were detected when the TIP1 or TIP2 particles were present but essentially no mCherry-expressing cells were detected in the control cells. FIG. 9B graphically illustrates the log 10 amount of SARS-CoV-2 genome when TIP1 and TIP2 therapeutic interfering particles were incubated with cells that were infected with SARS-CoV-2 for 24 hours compared to controls that were not infected by SARS-CoV-2. FIG. 9C graphically illustrates the log 10 amount of SARS-CoV-2 genome when TIP1 and TIP2 therapeutic interfering particles were incubated with cells that were infected with SARS-CoV-2 for 48 hours compared to controls that were not infected by SARS-CoV-2.

FIG. 10 schematically illustrates a method for interfering with SARS-CoV-2 transcription by transfection with antisense Transcription Regulating Sequences (TRS).

FIGS. 11A-11C graphically illustrate that antisense Transcription Regulating Sequences (TRS) can reduce SARS-CoV-2 plaque forming units (pfus). FIG. 11A graphically illustrates the SARS-CoV-2 pfu after transfection with antisense TRS1 (ACGAACCUAAACACGAACCUAAAC (SEQ ID NO:25)). FIG. 11B graphically illustrates the SARS-CoV-2 pfu after transfection with antisense TRS2 (ACGAACACGAACACGAACACGAAC (SEQ ID NO:26)). FIG. 11C graphically illustrates the SARS-CoV-2 pfu after transfection with antisense TRS3 (CUAAACCUAAACCUAAACCUAAAC (SEQ ID NO:27)).

FIG. 12 graphically illustrates that the combination of the TRS with either the TIP1 or the TIP2 significantly reduced the SARS-CoV-2 genome numbers compared to the TRS alone.

FIG. 13A-13C illustrate that TIP1 and TIP2 therapeutic interfering particles significantly reduce the replication of different SARS-CoV-2 strains, including South African and U.K. strains of SARS-CoV-2. FIG. 13A illustrates that TIP1 and TIP2 significantly reduce the replication of South African 501Y.V2.HV delta variant of SARS-CoV-2. FIG. 13B illustrates that TIP1 and TIP2 significantly reduce the replication of South African 501Y.V2.HV variant of SARS-CoV-2. FIG. 13C illustrates that TIP1 and TIP2 significantly reduce the replication of U.K B.1.1.7 variant of SARS-CoV-2.

### DETAILED DESCRIPTION

Described herein are methods for making defective SARS-CoV-2 particles that can interfere with SARS-CoV-2 infection (SARS-CoV-2 therapeutic interfering particles), and compositions of such interfering therapeutic particles useful for reducing SARS-CoV-2 infection.

As shown herein, SARS-CoV-2 therapeutic interfering particles (TIPs) can reduce SARS-CoV-2 replication by more than 50-fold. The SARS-CoV-2 TIPs can include segments of the 5′ and 3′ ends of the SARS-CoV-2 genome. For example, the SARS-CoV-2 TIPs can include segments of the 5′-UTR and the 3′-UTR of SARS-CoV-2. In some cases, a detectable marker and/or a barcode can be present between the 5′ and 3′ segments of the SARS-CoV-2 genome. Examples of SARS-CoV-2 therapeutic interfering particles (TIPs) include the TIP1, TIP2, TIP1*, and TIP2* constructs described herein.

The 5′ SARS-CoV-2 sequences in TIP1 are as shown below (SEQ ID NO:28).

```
    1 ATTAAAGGTT TATACCTTCC CAGGTAACAA ACCAACCAAC
  41 TTTCGATCTC TTGTAGATCT GTTCTCTAAA CGAACTTTAA
  81 AATCTGTGTG GCTGTCACTC GGCTGCATGC TTAGTGCACT
121 CACGCAGTAT AATTAATAAC TAATTAGTGT CGTTGACAGG
161 ACACGAGTAA CTCGTCTATC TTCTGCAGGC TGCTTACGGT
201 TTCGTCCGTG TTGCAGCCGA TCATCAGCAC ATCTAGGTTT
241 CGTCCGGGTG TGACCGAAAG GTAAGATGGA GAGCCTTGTC
281 CCTGGTTTCA ACGAGAAAAC ACACGTCCAA CTCAGTTTGC
321 CTGTTTTACA GGTTCGCGAC GTGCTCGTAC GTGGCTTTGG
361 AGACTCCGTG GAGGAGGTCT TATCAGAGGC ACGTCAACAT
401 CTTAAAGATG GCACTTGTGG CTTAGTAGAA GTTGAAAAAG
411 gcgttttgcc
```

The 3′ SARS-CoV-2 sequences in TIP1 are shown below as SEQ ID NO:29.

```
    1 GACCACACAA GGCAGATGGG CTATATAAAC GTTTTCGCTT
  41 TTCCGTTTAC GATATATAGT CTACTCTTGT GCAGAATGAA
  81 TTCTCGTAAC TACATAGCAC AAGTAGATGT AGTTAACTTT
121 AATCTCACAT AGCAATCTTT AATCAGTGTG TAACATTAGG
161 GAGGACTTGA AAGAGCCACC ACATTTTCAC CGAGGCCACG
201 CGGAGTACGA TCGAGTGTAC AGTGAACAAT GCTAGGGAGA
241 GCTGCCTATA TGGAAGAGCC CTAATGTGTA AAATTAATTT
281 TAGTAGTGCT ATCCCCATGT GATTTTAATA GCTTCTTAGG
321 AGAATGACAA AAAAAAAAAA AAAAAAAAAA AAAAAAAAAA
361 A
```

The 5′ SARS-CoV-2 sequences in TIP2 are as shown below (SEQ ID NO:30).

```
      1 ATTAAAGGTT TATACCTTCC CAGGTAACAA ACCAACCAAC
    41 TTTCGATCTC TTGTAGATCT GTTCTCTAAA CGAACTTTAA
    81 AATCTGTGTG GCTGTCACTC GGCTGCATGC TTAGTGCACT
  121 CACGCAGTAT AATTAATAAC TAATTACTGT CGTTGACAGG
  161 ACACGAGTAA CTCGTCTATC TTCTGCAGGC TGCTTACGGT
  201 TTCGTCCGTG TTGCAGCCGA TCATCAGCAC ATCTAGGTTT
  241 CGTCCGGGTG TGACCGAAAG GTAAGATGGA GAGCCTTGTC
  281 CCTGGTTTCA ACGAGAAAAC ACACGTCCAA CTCAGTTTGC
  321 CTGTTTTACA GGTTCGCGAC GTGCTCGTAC GTGGCTTTGG
  361 AGACTCCGTG GAGGAGGTCT TATCAGAGGC ACGTCAACAT
  401 CTTAAAGATG GCACTTGTGG CTTAGTAGAA GTTGAAAAAG
  441 GCGTTTTGCC TCAACTTGAA CAGCCCTATG TGTTCATCAA
  481 ACGTTCGGAT GCTCGAACTG CACCTCATGG TCATGTTATG
  521 GTTGAGCTGG TAGCAGAACT CGAAGGCATT CAGTACGGTC
  561 GTAGTGGTGA GACACTTGGT GTCCTTGTCC CTCATGTGGG
  601 CGAAATACCA GTGGCTTACC GCAAGGTTCT TCTTCGTAAG
  641 AACGGTAATA AAGGAGCTGG TGGCCATAGT TACGGCGCCG
  681 ATCTAAAGTC ATTTGACTTA GGCGACGAGC TTGGCACTGA
  721 TCCTTATGAA GATTTTCAAG AAAACTGGAA CACTAAACAT
  761 AGCAGTGGTG TTACCCGTGA ACTCATGCGT GAGCTTAACG
  801 GAGGGGCATA CACTCGCTAT GTCGATAACA ACTTCTGTGG
  841 CCCTGATGGC TACCCTCTTG AGTGCATTAA AGACCTTCTA
  881 GCACGTGCTG GTAAAGCTTC ATGCACTTTG TCCGAACAAC
  921 TGGACTTTAT TGACACTAAG AGGGGTGTAT ACTGCTGCCG
  961 TGAACATGAG CATGAAATTG CTTGGTACAC GGAACGTTCT
1001 GAAAAGAGCT ATGAATTGCA GACACCTTTT GAAATTAAAT
1041 TGGCAAAGAA ATTTGACACC TTCAATGGGG AATGTCCAAA
1081 TTTTGTATTT CCCTTAAATT CCATAATCAA GACTATTCAA
1121 CCAAGGGTTG AAAAGAAAAA GCTTGATGGC TTTATGGGTA
1161 GAATTCGATC TGTCTATCCA GTTGCGTCAC CAAATGAATG
1201 CAACCAAATG TGCCTTTCAA CTCTCATGAA GTGTGATCAT
1241 TGTGGTGAAA CTTCATGGCA GACGGGCGAT TTTGTTAAAG
1281 CCACTTGCGA ATTTTGTGGC ACTGAGAATT TGACTAAAGA
1321 AGGTGCCACT ACTTGTGGTT ACTTACCCCA AAATGCTGTT
1361 GTTAAAATTT ATTGTCCAGC ATGTCACAAT TCAGAAGTAG
1401 GACCTGAGCA TAGTCTTGCC GAATACCATA ATGAATCTGG
1441 CTTGAAAACC ATTCTTCGTA AGGGTGGTCG CACTATTGCC
1481 TTTGGAGGCT GTGTGTTCTC TTATGTTGGT TGCCATAACA
1521 AGTGTGCCTA TTGGGTTCCA gaattagatc tctcgaggtt
1561 aacgaattct gctatacgaa gttatccctc
```

The 3′ SARS-CoV-2 sequences in TIP2 are as shown below (SEQ ID NO:31).

```
    1 ATTTGCCCCC AGCGCTTCAG CGTTCTTCGG AATGTCGCGC
  41 ATTGGCATGG AAGTCACACC TTCGGGAACG TGGTTGACCT
  81 ACACAGGTGC CATCAAATTG GATGACAAAG ATCCAAATTT
121 CAAAGATCAA GTCATTTTGC TGAATAAGCA TATTGACGCA
161 TACAAAACAT TCCCACCAAC AGAGCCTAAA AAGGACAAAA
201 AGAAGAAGGC TGATGAAACT CAAGCCTTAG CGCAGAGACA
241 GAAGAAACAG CAAACTGTGA CTCTTCTTCC TGCTGCAGAT
281 TTGGATGATT TCTCCAAACA ATTGCAACAA TCCATGAGCA
321 GTGCTGACTC AACTCAGGCC TAAACTCATG CAGACCACAC
361 AAGGCAGATG GGCTATATAA ACGTTTTCGC TTTTCCGTTT
401 ACGATATATA GTCTACTCTT GTGCAGAATG AATTCTCGTA
441 ACTACATAGC ACAAGTAGAT GTAGTTAACT TTAATCTCAC
481 ATAGCAATCT TTAATCAGTG TGTAACATTA GGGAGGACTT
521 GAAAGAGCCA CCACATTTTC ACCGAGGCCA CGCGGAGTAC
561 GATCGAGTGT ACAGTGAACA ATGCTAGGGA GAGCTGCCTA
601 TATGGAAGAG CCCTAATGTG TAAAATTAAT TTTAGTAGTG
641 CTATCCCCAT GTGATTTTAA TAGCTTCTTA GGAGAATGAC
681 AAAAAAAAAA AAAAAAAAAA AAAAAAAAAA AAA
```

Two additional TIP variants were also cloned TIP11* and TIP2*, these contain the common C-241-T mutation within the 5′ UTR. This C241T UTR mutation co-transmits across populations together with the spike protein D614G mutation.

Hence, the 5′ SARS-CoV-2 sequences in TIP1* are as shown below (SEQ ID NO:32).

```
    1 ATTAAAGGTT TATACCTTCC CAGGTAACAA ACCAACCAAC
  41 TTTCGATCTC TTGTAGATCT GTTCTCTAAA CGAACTTTAA
  81 AATCTGTGTG GCTGTCACTC GGCTGCATGC TTAGTGCACT
121 CACGCAGTAT AATTAATAAC TAATTAGTGT CGTTGACAGG
161 ACACGAGTAA CTCGTCTATC TTCTGCAGGC TGCTTACGGT
201 TTCGTCCGTG TTGCAGCCGA TCATCAGCAC ATCTAGGTTT
241 TGTCCGGGTG TGACCGAAAG GTAAGATGGA GAGCCTTGTC
281 CCTGGTTTCA ACGAGAAAAC ACACGTCCAA CTCAGTTTGC
321 CTGTTTTACA GGTTCGCGAC GTGCTCGTAC GTGGCTTTGG
361 AGACTCCGTG GAGGAGGTCT TATCAGAGGC ACGTCAACAT
401 CTTAAAGATG GCACTTGTGG CTTAGTAGAA GTTGAAAAAG
411 GCGTTTTGCC
```

Similarly, the 5′ SARS-CoV-2 sequences in TIP2* are as shown below (SEQ ID NO:33).

```
      1 ATTAAAGGTT TATACCTTCC CAGGTAACAA ACCAACCAAC
    41 TTTCGATCTC TTGTAGATCT GTTCTCTAAA CGAACTTTAA
    81 AATCTGTGTG GCTGTCACTC GGCTGCATGC TTAGTGCACT
  121 CACGCAGTAT AATTAATAAC TAATTACTGT CGTTGACAGG
  161 ACACGAGTAA CTCGTCTATC TTCTGCAGGC TGCTTACGGT
  201 TTCGTCCGTG TTGCAGCCGA TCATCAGCAC ATCTAGGTTT
  241 TGTCCGGGTG TGACCGAAAG GTAAGATGGA GAGCCTTGTC
  281 CCTGGTTTCA ACGAGAAAAC ACACGTCCAA CTCAGTTTGC
  321 CTGTTTTACA GGTTCGCGAC GTGCTCGTAC GTGGCTTTGG
  361 AGACTCCGTG GAGGAGGTCT TATCAGAGGC ACGTCAACAT
  401 CTTAAAGATG GCACTTGTGG CTTAGTAGAA GTTGAAAAAG
  441 GCGTTTTGCC TCAACTTGAA CAGCCCTATG TGTTCATCAA
  481 ACGTTCGGAT GCTCGAACTG CACCTCATGG TCATGTTATG
  521 GTTGAGCTGG TAGCAGAACT CGAAGGCATT TCATGTTATG
  561 GTAGTGGTGA GACACTTGGT GTCCTTGTCC CTCATGTGGG
  601 CGAAATACCA GTGGCTTACC GCAAGGTTCT TCTTCGTAAG
  641 AACGGTAATA AAGGAGCTGG TGGCCATAGT TACGGCGCCG
  681 ATCTAAAGTC ATTTGACTTA GGCGACGAGC TTGGCACTGA
  721 TCCTTATGAA GATTTTCAAG AAAACTGGAA CACTAAACAT
  761 AGCAGTGGTG TTACCCGTGA ACTCATGCGT GAGCTTAACG
  801 GAGGGGCATA CACTCGCTAT GTCGATAACA ACTTCTGTGG
  841 CCCTGATGGC TACCCTCTTG AGTGCATTAA AGACCTTCTA
  881 GCACGTGCTG GTAAAGCTTC ATGCACTTTG TCCGAACAAC
  921 TGGACTTTAT TGACACTAAG AGGGGTGTAT ACTGCTGCCG
  961 TGAACATGAG CATGAAATTG CTTGGTACAC GGAACGTTCT
1001 GAAAAGAGCT ATGAATTGCA GACACCTTTT GAAATTAAAT
1041 TGGCAAAGAA ATTTGACACC TTCAATGGGG AATGTCCAAA
1081 TTTTGTATTT CCCTTAAATT CCATAATCAA GACTATTCAA
1121 CCAAGGGTTG AAAAGAAAAA GCTTGATGGC TTTATGGGTA
1161 GAATTCGATC TGTCTATCCA GTTGCGTCAC CAAATGAATG
1201 CAACCAAATG TGCCTTTCAA CTCTCATGAA GTGTGATCAT
1241 TGTGGTGAAA CTTCATGGCA GACGGGCGAT TTTGTTAAAG
1281 CCACTTGCGA ATTTTGTGGC ACTGAGAATT TGACTAAAGA
1321 AGGTGCCACT ACTTGTGGTT ACTTAGCCCA AAATGCTGTT
1361 GTTAAAATTT ATTGTCCAGC ATGTCACAAT TCAGAAGTAG
1401 GACCTGAGCA TAGTCTTGCC GAATAGCATA ATGAATCTGG
1441 CTTGAAAACC ATTCTTCGTA AGGGTGGTCG CACTATTGCC
1481 TTTGGAGGCT GTGTGTTCTC TTATGTTGGT TGCCATAACA
1521 AGTGTGCCTA TTGGGTTCCA gaaatagatc tctcgaggtt
1561 aacgaattct gctatacgaa gttatccctc
```

The TIP constructs used in the experiments described herein included a marker (mCherry) encoded between the 5′ and 3′ SARS-CoV-2 nucleic acids. Expression of such a marker allowed replication the TIP constructs to be detected in cells transfected with the TIP constructs. Inclusion of such markers is useful for monitoring the TIPs but the marker may not be needed or included in therapeutic interfering particles that are administered as treatment of a patient or subject infected with SARS-CoV-2.

In general, the methods for making SARS-CoV-2 therapeutic interfering particles involve cleaving a population of circular SARS-CoV-2 DNA at different positions in the DNA circle to generate a library of cleaved (linearized) SARS-CoV-2 DNAs where members of the library are cut at different locations. One or more exonucleases are then used to ‘chew back’ the end(s) of the cut site and the ‘chewed ends’ are then ligated to reform circular DNA. This generates a deletion library. There are numerous ways to achieve each of the steps (e.g., the cleavage step at different positions for the members of the library), and there are optional steps that can be performed prior to the circularizing (e.g., ligation) step. As discussed in more detail below, more than one round of library generation can be performed, and thus the subject methods can be used the generate complex deletion libraries in which members of the library include more than one deletion.

### Generating a Library of Cleaved (Linearized) SARS-CoV-2 DNAs

The methods described herein include generating a library of cleaved (linearized) SARS-CoV-2 DNAs from a population of circular SARS-CoV-2 DNAs. In some cases, the position of cleavage of the SARS-CoV-2 DNA population is random. For example, a transposon cassette can be inserted at random positions into a population of SARS-CoV-2 DNAs, where the transposon cassette includes a target sequence (recognition sequence) for a sequence specific DNA endonuclease. In such a case, the transposon cassette is being used as a vehicle for inserting a recognition sequence into the population of SARS-CoV-2 DNAs (at random positions). A sequence specific DNA endonuclease (one that recognizes the recognition sequence) can then be used to cleave the SARS-CoV-2 DNAs, thereby generating a library of cleaved (linearized) SARS-CoV-2 DNAs where members of the library are cut at different locations.

The term “transposon cassette” is used herein to mean a nucleic acid molecule that includes a ‘sequence of interest’ flanked by sequences that can be used by a transposon to insert the sequence of interest into a SARS-CoV-2 DNA. Thus, in some cases, the ‘sequence of interest’ is flanked by transposon compatible inverted terminal repeats (ITRs), i.e., ITRs that are recognized and utilized by a transposon. In cases where a transposon cassette is used as a vehicle for inserting one or more target sequences (for one or more sequence specific DNA endonucleases) into SARS-CoV-2 DNAs, the sequence of interest can include the one or more recognition sequences.

In some cases, the sequence of interest includes a selectable marker gene, for example, a nucleotide sequence encoding a selectable marker such as a gene encoding a protein that provides for drug resistance, for example, antibiotic resistance. In some cases, a sequence of interest includes a first copy and a second copy of a recognition sequence for a first sequence specific DNA endonuclease (e.g., a first meganuclease). In some cases, a sequence of interest includes a selectable marker gene flanked by a first and second recognition sequence for a sequence specific DNA endonuclease (e.g., meganuclease). In some such cases, the first recognition sequence and the second recognition sequence are identical and can be considered a first copy and a second copy of a recognition sequence. In some such cases, the first recognition sequence is different than the second recognition sequence. In some cases, the first recognition sequence and second recognition sequence (e.g., first and second copies of a recognition sequence) flank a selectable marker gene, for example, one that encodes a drug resistance protein such as an antibiotic resistance protein. In some embodiments, a subject transposon cassette includes a first copy and a second copy of a recognition sequence for a first meganuclease; and a first copy and a second copy of a recognition sequence for a second meganuclease.

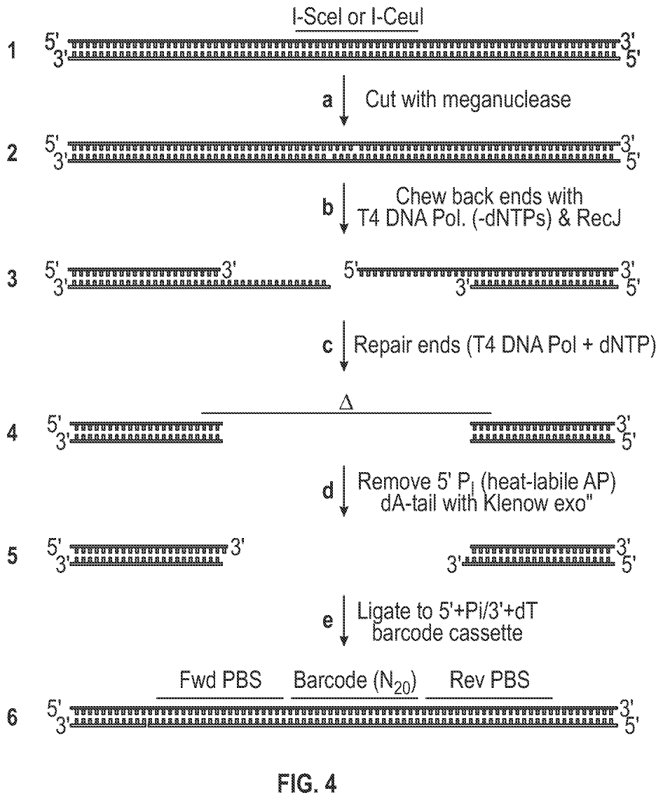

In any of the above scenarios, in some cases, the first and/or second recognition sequence is a site for 1-Sce1 meganuclease (e.g., aactataacggtcctaa\hat{ }ggtagcgaa (SEQ ID NO:34)). In some cases, the first and/or second recognition sequence is a site for 1-Ceu1 meganuclease (e.g., aactataacggtcctaa\hat{ }ggtagcgaa (SEQ ID NO:35)). See. FIG. 4. In some cases, a first recognition sequence is a site for 1-Sce1 and a second recognition sequence is a site for 1-Ceu1. In some cases a first and/or second recognition sequence is a recognition sequence for a meganuclease, for example, selected from: a LAGLIDADG meganuclease (LMNs), 1-Sce1, 1-Ceu1, 1-Cre1, 1-Dmo1, 1-Chu1, 1-Dir1, 1-Flmu1, 1-Flmu11, 1-Ani1, 1-Sce1V, 1-Csm1, 1-Pan1, 1-Pan11, 1-PanMI, 1-Sce11, I-Ppo1, 1-Sce111, 1-Ltd, 1-Gpi1, 1-GZe1, 1-Onu1, 1-HjeMI, I-Mso1, 1-Tev1, 1-Tev11, 1-Tev111, P1-Mie1, P1-Mtu1, P1-Psp1, P1-Tli I, P1-Tli II, and P1-SceV.

As noted above, a subject transposon cassette includes a sequence of interest flanked by transposase compatible inverted terminal repeats (ITRs). The ITRs can be compatible with any desired transposase, for example, a bacterial transposase such as Tn3, Tn5, Tn7, Tn9, Tn10, Tn903, Tn1681, and the like; and eukaryotic transposases such as Tc1/mariner super family transposases, piggyBac superfamily transposases, hAT superfamily transposases, Sleeping Beauty, Frog Prince, Minos, Himari, and the like. In some cases, the transposase compatible ITRs are compatible with (i.e., can be recognized and utilized by) a Tn5 transposase. Some of the methods provided herein include a step of inserting a transposase cassette into a SARS-CoV-2 DNA. Such a step includes contacting the SARS-CoV-2 DNA and the transposon cassette with a transposase. In some cases, this contacting occurs inside of a cell such as a bacterial cell, and in some cases this contacting occurs in vitro outside of a cell. As the transposase compatible ITRs listed above are suitable for compositions and methods disclosed herein, so too are the transposases. As such, suitable transposases include but are not limited to bacterial transposases such as Tn3, Tn5, Tn7, Tn9, Tn10, Tn903, Tn1681, and the like; and eukaryotic transposases such as Tc1/mariner super family transposases, piggyBac superfamily transposases, hAT superfamily transposases, Sleeping Beauty, Frog Prince, Minos, Himarl, and the like. In some cases, the transposase is a Tn5 transposase.

In some embodiments, a subject method includes a step of inserting a target sequence (e.g., one or more target sequences) for a sequence specific DNA endonuclease (e.g., one or more sequence specific DNA endonucleases) into a population of circular SARS-CoV-2 DNAs, thereby generating a population of sequence-inserted circular SARS-CoV-2 DNAs. In some cases, the inserting step is carried out by inserting a transposon cassette that includes the target sequence (e.g., the one or more target sequences), thereby generating a population of transposon-inserted circular SARS-CoV-2 DNAs. In some cases, the transposon cassette includes a single recognition sequence (e.g., in the middle or near one end of the transposon cassette) and can therefore be used to introduce a single recognition sequence into the population of SARS-CoV-2 DNAs. In some cases, the transposon cassette includes more than one recognition sequences (e.g., a first and a second recognition sequence). In some such cases, the first and second recognition sequences are positioned at or near the ends of the transposon cassette (e.g., within 20 bases, 30 bases, 50 bases, 60 bases, 75 bases, or 100 bases of the end) such that cleavage of the first and second recognition sequences effectively removes the transposon cassette (or most of the transposon cassette) from the SARS-CoV-2 DNA, while simultaneously generating a linearized SARS-CoV-2 DNA, and therefore generating the desired library of cleaved (linearized) SARS-CoV-2 DNAs where members of the library are cut at different locations.

In some cases when the transposon cassette include first and second recognition sequences, the first and second recognition sequences are the same, and are therefore first and second copies of a given recognition sequence. In some such cases, the same sequence specific DNA endonuclease (e.g., restriction enzyme, meganuclease, programmable genome editing nuclease) can then be used to cleave at both sites.

In some embodiments, the transposon cassette includes a first and a second recognition sequence where the first and second recognition sequences are not the same. In some such cases, a different sequence specific DNA endonuclease (e.g., restriction enzyme, meganuclease, programmable genome editing nuclease) is used to cleave the two sites (e.g., the library of transposon-inserted SARS-CoV-2 DNAs can be contacted with two sequence specific DNA endonucleases). However, in some cases one sequence specific DNA endonuclease can still be used. For example, in some cases two different guide RNAs can be used with the same CRISPR/Cas protein. As another example, in some cases a given sequence specific DNA endonuclease can recognize both recognition sequences.

In some cases, the population of circular SARS-CoV-2 DNAs (e.g., plasmids) are present inside of host cells (e.g., bacterial host cells such as E. coli) and the step of inserting a transposon cassette takes place inside of the host cell. For example, the methods can include introducing a transposase and/or a nucleic acid encoding a transposase into a selected cell or expression of a transposase within the cell from an existing expression cassette that encodes the transposase, and the like. In some such cases, a subject method can include a selection/growth step in the host cell. For example, if the transposon cassette includes a drug resistance marker, the host cells can be grown in the presence of drug to select for those cells harboring a transposon-inserted circular target DNA.

Once a population of transposon-inserted circular SARS-CoV-2 DNAs is generated (and in some cases after a selection/growth step in the host cells), the population can be isolated/purified from the host cells prior to the next step (e.g., prior to contacting them with a sequence specific DNA endonuclease).

Because the circular SARS-CoV-2 DNAs can be small circular DNAs (e.g., less than 50 kb), a selection and growth step in bacteria can in some cases be avoided through the use of in vitro rolling circle amplification (RCA). For example, after repair of nicked target DNA post-transposition, a highly-processive and strand-displacing polymerase (e.g., phi29 DNA polymerase), along with primers specific to the inserted transposon cassette, can be used to selectively amplify insertion mutants from the pool of circular plasmids. In other words, such a step can circumvent amplifying DNA through bacterial transformation. Use of RCA can decrease the time required for growth/selection of bacteria and can avoid biasing the library towards clones that do not impede bacterial growth.

### Non-Random Cleavage

As noted above, in some cases the position of cleavage of the SARS-CoV-2 DNA population is random, however in some cases the position of cleavage is not random. For example, a population of SARS-CoV-2 DNAs can be distributed (e.g., aliquoted) into different vessels (e.g., different tubes, different wells of a multi-well plate etc.). If a specific sequence of interest is selected within the SARS-CoV-2 genomic sequence, then that sequence of interest can be cleaved within the circular SARS-CoV-2 DNAs. Separate aliquots of circular SARS-CoV-2 DNAs can be placed within different vessels (e.g., wells of the multi-well plate) and the different aliquots of circular SARS-CoV-2 DNAs can be cleaved at different pre-determined locations by using a programmable sequence specific endonuclease. For example, if a CRISPR/Cas endonuclease (e.g., Cas9, Cpf1, and the like) is used, guide RNAs can readily be designed to target any desired sequence within the SARS-CoV-2 genome (e.g., while taking protospacer adjacent motif (PAM) sequence requirements into account in some cases). For example, guide RNAs can be tiled at any desired spacing along the circular SARS-CoV-2 DNAs (e.g., every 5 nucleotides (nt), every 10 nt, every 20 nt, every 50nt—overlapping, non-overlapping, and the like). The circular SARS-CoV-2 DNAs in each vessel (e.g., each well) can be contacted with one of the guide RNAs in addition to the CRISPR/Cas endonuclease. In this way, a library of cleaved SARS-CoV-2 DNAs can be generated where members of the library are separated from one another because they are in separate vessels. As would be understood by one of ordinary skill in the art, in some cases, one would take PAM sequences into account when designing guide RNAs, and therefore the spacing between guide RNA target sites can be a function of PAM sequence constraints, and consistent spacing across a given target sequence would not necessarily be possible in some cases. However, different CRISPR/Cas endonucleases (e.g., even the same protein, such as Cas9, isolated from different species) can have different PAM requirements, and thus, the use of more than one CRISPR/Cas endonuclease can in some cases relieve at least some of the constraints imposed by PAM requirements on available target sites. Further steps of the method can then be carried out separately (e.g., in separate vessels, in separate wells of a multi-well plate), or at any step, members can be pooled and treated together in one vessel.

As an illustrative but non-limiting example, one could use 96 different guide RNAs (or 384 different guide RNAs) to cleave aliquots of circular SARS-CoV-2 DNAs in 96 different wells of a 96-well plate (or 384 different wells of a 384 well plate), to generate 96 members (or 384 members) of a library where each member is cleaved at a different site. The cleavage sites can be designed by the user prior to starting the method. The exonuclease step (chew back) can then be performed in separate wells (e.g., by aliquoting exonuclease to each well), or two more wells can be pooled prior to adding exonuclease to the pool.

### Circular SARS-CoV-2 DNAs

A circular SARS-CoV-2 DNA of a population of circular SARS-CoV-2 DNAs can be any circular SARS-CoV-2 DNA and can be generated from any isolate of SARS-CoV-2. In some cases, the circular SARS-CoV-2 DNAs are plasmid DNAs. For example, in some cases, the circular SARS-CoV-2 DNAs include an origin of replication (ORI). In some cases, the circular SARS-CoV-2 DNAs include a drug resistance marker (e.g., a nucleotide sequence encoding a protein that provides for drug resistance). In some embodiments, a population of circular SARS-CoV-2 DNAs are generated from a population of linear DNA molecules (e.g., via intramolecular ligation). For example, a subject method can include a step of circularizing a population of linear SARS-CoV-2 DNA molecules (e.g., a population of PCR products, a population of linear viral SARS-CoV-2 genomes, a population of products from a restriction digest, etc.) to generate a population of circular SARS-CoV-2 DNAs. In some cases, members of such a population are identical (e.g., many copies of a PCR product or restriction digest can be used to generate a population of SARS-CoV-2 DNAs, where each circular DNA is identical). In some cases, members of such a population of circular SARS-CoV-2 DNAs can be different from one another. For example, the population of circular SARS-CoV-2 DNAs can be generated from two or more different SARS-CoV-2 isolates or be generated from different SARS-CoV-2 PCR products or be generated from different restriction digest products of SARS-CoV-2.

In some cases, the population of circular SARS-CoV-2 DNAs can itself be a deletion library. For example, the population of circular SARS-CoV-2 DNAs can be a library of known deletion mutants (e.g., known viral deletion mutants). As another example, if two rounds of a subject method are performed, the starting population of SARS-CoV-2 DNAs for the second round can be a deletion library (e.g., generated during a first round of deletion) where members of the library include deletions of different sections of DNA relative to other members of the library. Such a library can serve as a population of circular SARS-CoV-2 DNAs, e.g., a transposon cassette can still be introduced into the population. Performing a second round of deletion in this manner can therefore generate constructs with deletions at multiple different entry points. As an illustrative example, for a SARS-CoV-2 DNA of about 29-30 kb (kilobases) in length, the first round of deletion might have deleted bases 2000 through 2650 for a one member (of the library that was generated), of which multiple copies would likely be present. A second round of deletion might generate two new members, both of which are generated from copies of the same deletion member. Thus, for example, one new member might be generated with bases 3500 through 3650 deleted (in addition to bases 2000 through 2650), while a second new member might be generated with bases 1500 through 1580 deleted (in addition to bases 2000 through 2650). Thus, multiple rounds of deletion (e.g., 2, 3, 4, 5, etc.) can produce complex deletion libraries. In some cases, more than one round of library generation is performed where the second round includes the insertion of a transposon cassette, e.g., as described above.

For example, in some cases, a first round of deletion is performed using a CRISPR/Cas endonuclease to generate the cleaved linear SARS-CoV-2 DNAs by targeting the CRISPR/Cas endonuclease to pre-selected sites within the population of circular SARS-CoV-2 DNAs (e.g., by designing guide RNAs, e.g., at pre-selected spacing, to target one or more SARS-CoV-2 sequences of interest). After exonuclease treatment and circularization to generate a first library of circularized deletion DNAs, the library of circularized deletion DNAs is used as input (as a population of circular SARS-CoV-2 DNAs) for a second round of deletion. Thus, one or more target sequences for one or more sequence specific DNA endonucleases (e.g., one or more meganucleases) is inserted (e.g., at random positions via a transposon cassette) into the library of circularized SARS-CoV-2 deletion DNAs to generate a population of transposon-inserted circular SARS-CoV-2 DNAs, and the method is continued. In some such cases, the first round of deletion might only target a small number of locations of interest for deletion (one location, e.g., using only one guide RNA that targets a particular location; or a small number of locations, e.g., using a small number of guide RNAs to target a small number of locations), while the second round is used to generate deletion constructs that include the first deletion plus a second deletion.

In some cases, the circular SARS-CoV-2 DNAs include the whole viral genome. In other cases, the circular SARS-CoV-2 DNAs include a partial SARS-CoV-2 viral genome. Thus, in some cases the subject methods are used to generate a library of viral deletion mutants. In some such cases, a library of generated viral deletion mutants can be considered a library of potential defective interfering particles (DIPs). DIPs are mutant versions of SARS-CoV-2 viruses that include genomic deletions such that they are unable to replicate except when complemented by wild-type virus replicating within the same cell. Defective interfering particles (DIPs) can arise naturally because viral genomes encode both cis-acting and trans-acting elements. Trans-acting elements (trans-elements) code for gene products, such as capsid proteins or transcription factors, and cis-acting elements (cis-elements) are regions of the viral genome that interact with trans-element products to achieve productive viral replication including viral genome amplification, encapsidation, and viral egress. In other words, the SARS-CoV-2 viral genome of a DIP can still be copied and packaged into viral particles if the missing (deleted) trans-elements are provided in trans (e.g., by a co-infecting virus). In some cases, a DIP can be used therapeutically to reduce viral infectivity of a co-infecting virus, e.g., by competing for and therefore diluting out the available trans-elements. In such cases, where a SARS-CoV-2 DIP can be used as a therapeutic (e.g., as a treatment for Covid-19 infections), that SARS-CoV-2 DIP can be referred to as a therapeutic interfering particle (TIP).

While DIPs may arise naturally, methods of this disclosure can be used to generate useful types of SARS-CoV-2 DIPs, for example, by generating a deletion library of viral SARS-CoV-2 genomes. DIPs can then be identified from such a deletion library by sequencing the library members to identify those predicted to be DIPs. Alternatively, or additionally, a generated deletion library can be screened. For example, a library of SARS-CoV-2 DIPs can be introduced into cells, to identify those members with viral genomes having the desired function. Additional description of DIPs and TIPs and uses thereof is provided in U.S. Patent Application Publication No. 20160015759, the disclosure of which is incorporated by reference herein in its entirety.

Thus, in some cases a subject method includes introducing members of a library of generated SARS-CoV-2 deletion constructs into a target cell (e.g., a eukaryotic cell, such as a mammalian cell, such as a human cell) and assaying for infectivity. In some such cases, the assaying step also includes complementation of the library members with a co-infecting SARS-CoV-2 virus.

Such introducing is meant herein to encompass any form of introduction of nucleic acids into cells (e.g., electroporation, transfection, lipofection, nanoparticle delivery, viral delivery, and the like). For example, such ‘introduction’ encompasses infecting mammalian cells in culture (e.g., with members of a generated library of circularized SARS-CoV-2 deletion viral DNAs that can be encapsulated as viral particles that contain viral genomes encoded by the members of the generated library of circularized deletion viral DNAs).

In some cases, a method includes generating from a library of SARS-CoV-2 deletion DNAs, at least one of: linear double stranded DNA (dsDNA) products, linear single stranded DNA (ssDNA) products, linear single stranded RNA (ssRNA) products, and linear double stranded RNA (dsRNA) products. Thus in some such cases, a subject method includes introducing such linear dsDNA products, linear ssDNA products, linear ssRNA products, and/or linear dsRNA products into mammalian cells (e.g., via any convenient method for introducing nucleic acids into cells, including but not limited to electroporation, transfection, lipofection, nanoparticle delivery, viral delivery, and the like).

Such methods can also include assaying for viral infectivity. Assaying for viral infectivity can be performed using any convenient method. Assaying for viral infectivity can be performed on the cells into which the members of the library of circularized SARS-CoV-2 deletion DNAs (and/or at least one of: linear double stranded DNA (dsDNA) products, linear single stranded DNA (ssDNA) products, linear single stranded RNA (ssRNA) products, and linear double stranded RNA (dsRNA) products generated from the library of circularized deletion DNAs) are introduced. For example, in some cases the members and/or products are introduced as encapsulated particles. In some cases, members of the library of circularized SARS-CoV-2 deletion DNAs (and/or at least one of: linear dsDNA products, linear ssDNA products, linear ssRNA products, and linear dsRNA products generated from the library of circularized SARS-CoV-2 deletion DNAs) are introduced into a first population of cells (e.g., mammalian cells) in order to generate viral particles, and the viral particles are then used to contact a second population of cells (e.g., mammalian cells). Thus, as used herein, unless otherwise explicitly described, the phrase “assaying for viral infectivity” encompasses both of the above scenarios (e.g., encompasses assaying for infectivity in the cells into which the members and/or products were introduced, and also encompasses assaying the second population of cells as described above).

In some embodiments a subject method (e.g., a method of generating and identifying a DIP) includes, after generating a deletion library (e.g., a library of circularized SARS-CoV-2 deletion DNAs), a high multiplicity of infection (MOI) screen (e.g., utilizing a MOI of >2). As used herein, a “high MOI” is a MOI of 2 or more (e.g., 2.5 or more, 3 or more, 5 or more, etc.). In some cases, a subject method uses a high MOI. Thus, in some cases, a subject method uses a MOI (a high MOI) of 2 or more, 3 or more, or 5 or more. In some cases, a subject method uses a MOI (a high MOI) in a range of from 2-150 (e.g., from 2-100, 2-80, 2-50, 2-30, 3-150, 3-100, 3-80, 3-50, 3-30, 5-150, 5-100, 5-80, 5-50, or 5-30). In some cases, a subject method uses a MOI (a high MOI) in a range of from 3-100 (e.g., 5-100). At high MOI, many (if not all) cells are infected by more than one virus, which allows for complementation of defective viruses by wildtype counterparts. Repeated passaging of deletion mutant libraries at high-MOI can select for mutants that can be mobilized effectively by a wild type SARS-CoV-2. For example, in some cases the method includes infecting mammalian cells in culture with members of the library of circularized SARS-CoV-2 deletion viral DNAs at a high multiplicity of infection (MOI), culturing the infected cells for a period of time ranging from 12 hours to 2 days (e.g., from 12 hours to 36 hours or 12 hours to 24 hours), adding naive cells to the to the culture, and harvesting virus from the cells in culture. However, this screening step can in some cases select for DIPs/TIPs which can be mobilized effectively by the wildtype virus but are cytopathic in the absence of the wildtype coinfection.

Thus, in some embodiments a subject method (e.g., a method of generating and identifying a DIP) includes a more stringent screen (referred to herein as a “low multiplicity of infection (MOI) screen”). As used herein, a “low MOI” includes use of a MOI of less than 1 (e.g., less than 0.8, less than 0.6, etc.). In some cases, a subject method uses a low MOI. Thus, in some cases, a subject method uses a MOI (a low MOI) of less than 1 (e.g., less than 0.8, less than 0.6). In some cases, a subject method uses a MOI (a low MOI) in a range of from 0.001-0.8 (e.g., from 0.001-0.6, 0.001-0.5, 0.005-0.8, 0.005-0.6, 0.01-0.8, or 0.01-0.5). In some cases, a subject method uses a MOI (a low MOI) in a range of from 0.01-0.5. For example, a low-MOI infection of target cells with a deletion library (e.g., utilizing a MOI of <1) can be alternated with a high-MOI infection of the transduced population with wildtype virus (e.g., SARS-CoV-2) to mobilize DIPs to naive cells.

In some cases, cells with one or more SARS-CoV-2 or one or more SARS-CoV-2 deletion DNAs can be propagated in the presence of a drug to test whether further rounds of replication occur. During the recovery period, cells infected with wild type virus (e.g., SARS-CoV-2 infected cells) will be killed, but cells transduced by well-behaving mutants (which do not produce cell-killing trans-factors) will be maintained. In this fashion, mutants can be selected that do not kill their transduced host-cell but that can mobilize during wildtype virus coinfection. Thus, in some cases a subject method includes infecting mammalian cells in culture with members of the library of circularized deletion SARS-CoV-2 viral DNAs at a low multiplicity of infection (MOI), culturing the infected cells in the presence of an inhibitor of viral replication for a period of time ranging from 1 day to 6 days (e.g., from 1 day to 5 days, from 1 day to 4 days, from 1 day to 3 days, or from 1 day to 2 days), infecting the cultured cells with functional SARS-CoV-2 virus at a high MOI, culturing the infected cells for a period of time ranging from 12 hours to 4 days (e.g., 12 hours to 72 hours, 12 hours to 48 hours, or 12 hours to 24 hours), and harvesting virus from the cultured cells.

In some embodiments, a subject method includes (a) inserting a target sequence for a sequence specific DNA endonuclease into a population of circular SARS-CoV-2 viral DNAs, to generate a population of sequence-inserted SARS-CoV-2 DNAs; (b) contacting the population of sequence-inserted SARS-CoV-2 DNAs with the sequence specific DNA endonuclease to generate a population of cleaved linear SARS-CoV-2 DNAs; (c) contacting the population of cleaved linear viral DNAs with an exonuclease to generate a population of SARS-CoV-2 deletion DNAs; (d) circularizing (e.g., via ligation) the SARS-CoV-2 deletion DNAs to generate a library of circularized SARS-CoV-2 deletion DNAs; and (e) sequencing members of the library of circularized SARS-CoV-2 deletion DNAs to identify deletion interfering particles (DIPs). In some cases, the method includes inserting a barcode sequence prior to or simultaneous with step (d).

In some cases, the inserting of step (a) includes inserting a transposon cassette into the population of circular SARS-CoV-2 viral DNAs, wherein the transposon cassette includes the target sequence for the sequence specific DNA endonuclease, and where the generated population of sequence-inserted SARS-CoV-2 DNAs is a population of transposon-inserted viral DNAs. In some cases (e.g., in some cases when using a CRISPR/Cas endonuclease), a subject method does not include step (a), and the first step of the method is instead cleaving members of the library in different locations relative to one another, which step can be followed by the exonuclease step.

### Target Sequence and Sequence Specific DNA Endonucleases

In some cases, a target sequence for a sequence specific DNA endonuclease is inserted into a SARS-CoV-2 DNA, for example, using a transposon cassette. The ‘target sequence’ is also referred to herein as a recognition sequence or recognition site. The term sequence specific endonuclease is used herein to refer to a DNA endonuclease that binds to and/or recognizes the target sequence in a SARS-CoV-2 DNA and cleaves the SARS-CoV-2 DNA. In other words, a sequence specific DNA endonuclease recognizes a specific sequence (a recognition sequence) within a SARS-CoV-2 DNA molecule and cleaves the molecule based on that recognition. In some cases, the sequence specific DNA endonuclease cleaves the SARS-CoV-2 DNA within the recognition sequence and in some cases it cleaves outside of the recognition sequence (e.g., in the case of type US restriction endonucleases).

The term sequence specific DNA endonuclease encompasses can include, for example, restriction enzymes, meganucleases, and programmable genome editing nucleases. Examples of sequence specific endonucleases include but are not limited to: restriction endonucleases such as EcoRI, EcoRV, BamHI, etc.; meganucleases such as LAGLI DADG meganucleases (LMNs), 1-Sce1, 1-Ceu1, 1-Cre1, 1-Dmo1, 1-Chu1, 1-Dir1, 1-Flmu1, 1-Flmu11, 1-Ani1, 1-Sce1V, 1-Csm1, 1-Pan1, 1-Pan11, 1-PanMI, 1-Sce11, 1-Ppo1, 1-Sce111, 1-Ltr1, 1-Gpi1, 1-GZe1, 1-Onu1, 1-HjeMI, 1-Mso1, 1-Tev1, 1-Tev11, 1-Tev111, P1-Mle1, P1-Mtu1, P1-Psp1, PI-TIi I, PI-TIi II, P1-SceV, and the like; and programmable gene editing endonucleases such as Zinc Finger Nucleases (ZFNs), transcription activator like effector nuclease (TALENs), and CRISPR/Cas endonucleases. In some cases, the sequence specific endonuclease of a subject composition and/or method is selected from: a meganuclease and a programmable gene editing endonuclease. In some cases, the sequence specific endonuclease of a subject composition and/or method is selected from: a meganuclease, a ZFN, a TALEN, and a CRISPR/Cas endonuclease (e.g., Cas9, Cpf1, and the like).

In some cases, the sequence specific endonuclease of a subject composition and/or method is a meganuclease. In some cases the meganuclease is selected from: LAGLIDADG meganucleases (LMNs), 1-Sce1, 1-Ceu1, 1-Cre1, 1-Dmo1, 1-Chu1, 1-Dir1, 1-Flmu1, 1-Flmu11, 1-Ani1, I-Sce1V, 1-Csm1, 1-Pan1, 1-Pan11, 1-PanMI, 1-Sce11, 1-Ppo1, 1-Sce111, 1-Ltr1, 1-Gpi1, 1-GZe1, 1-Onu1, I-HjeMI, 1-Mso1, 1-Tev1, 1-Tev11, 1-Tev111, P1-Mle1, P1-Mtu1, P1-Psp1, PI-TIi I, PI-TIi II, and P1-SceV. In some cases, the meganuclease 1-Sce1 is used. In some cases, the meganuclease 1-Ceu1 is used. In some cases, the meganucleases 1-Sce1 and 1-Ceu1 are used.

In some cases, the sequence specific DNA endonuclease is a programmable genome editing nuclease. The term “programmable genome editing nuclease” is used herein to refer to endonucleases that can be targeted to different sites (recognition sequences) within a SARS-CoV-2 DNA. Examples of suitable programmable genome editing nucleases include but are not limited to zinc finger nucleases (ZFNs), TAL-effector DNA binding domain-nuclease fusion proteins (transcription activator-like effector nucleases (TALENs)), and CRISPR/Cas endonucleases (e.g., class 2 CRISPR/Cas endonucleases such as a type II, type V, or type VI CRISPR/Cas endonucleases). Thus, in some embodiments, a programmable genome editing nuclease is selected from: a ZFN, a TALEN, and a CRISPR/Cas endonuclease (e.g., a class 2 CRISPR/Cas endonuclease such as a type II, type V, or type VI CRISPR/Cas endonuclease). In some cases, the sequence specific endonuclease of a subject composition and/or method is a CRISPR/Cas endonuclease (e.g., Cas9, Cpf1, and the like). In some cases, the sequence specific endonuclease of a subject composition and/or method is selected from: a meganuclease, a ZFN, and a TALEN.

Information related to class 2 type II CRISPR/Cas endonuclease Cas9 proteins and Cas9 guide RNAs (as well as methods of their delivery) (as well as information regarding requirements related to protospacer adjacent motif (PAM) sequences present in SARS-CoV-2 nucleic acids) can be found, for example, in the following Jinek et al., Science. 2012 Aug. 17; 337(6096):816-21; Chylinski et al., RNA Biol. 2013 May; 10(5): 726-37; Ma et al., Biomed Res Int. 2013; 2013:270805; Hou et al., Proc Natl Acad Sci USA. 2013 Sep. 24; 1 10(39): 15644-9; Jinek et al., Elife. 2013; 2:e00471; Pattanayak et al., Nat Biotechnol. 2013 September; 31 (9):839-43; Qi et al, Cell. 2013 Feb. 28; 152(5): 1173-83; Wang et al., Cell. 2013 May 9; 153(4):910-8; Auer et. al., Genome Res. 2013 Oct. 31; Chen et. al., Nucleic Acids Res. 2013 Nov. 1; 41 (20):e19; Cheng et. al., Cell Res. 2013 October; 23(10): 1 163-71; Cho et. al., Genetics. 2013 November; 195(3): 1 177-80; DiCarlo et al., Nucleic Acids Res. 2013 April; 41 (7):4336-43; Dickinson et. al., Nat Methods. 2013 October; 10(10): 1028-34; Ebina et. al., Sci Rep. 2013; 3:2510; Fujii et. al, Nucleic Acids Res. 2013 Nov. 1; 41 (20):e187; Hu et. al., Cell Res. 2013 November; 23(1 1): 1322-5; Jiang et. al., Nucleic Acids Res. 2013 Nov. 1; 41 (20):e188; Larson et. al., Nat Protoc. 2013 November; 8(1 1):2180-96; Mali et. at., Nat Methods. 2013 October; 10(10):957-63; Nakayama et. al., Genesis. 2013 December; 51 (12):835-43; Ran et. al., Nat Protoc. 2013 November; 8(1 1):2281-308; Ran et. al., Cell. 2013 Sep. 12; 154(6): 1380-9; Upadhyay et. al., G3 (Bethesda). 2013 Dec. 9; 3(12):2233-8; Walsh et. al., Proc Natl Acad Sci USA. 2013 Sep. 24; 110(39): 15514-5; Xie et. al., Mol Plant. 2013 Oct. 9; Yang et. al., Cell. 2013 Sep. 12; 154(6): 1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety. Examples and guidance related to type V CRISPR/Cas endonucleases (e.g., Cpf1) or type VI CRISPR/Cas endonucleases and guide RNAs (as well as information regarding requirements related to protospacer adjacent motif (PAM) sequences present in SARS-CoV-2 nucleic acids) can be found in the art, for example, see Zetsche et al, Cell. 2015 Oct. 22; 163(3):759-71; Makarova et al, Nat Rev Microbiol. 2015 November; 13(11):722-36; and Shmakov et al., Mol Cell. 2015 Nov. 5; 60(3):385-97.

Useful designer zinc finger modules include those that recognize various GNN and ANN triplets (Dreier, et al., (2001) J Biol Chem 276:29466-78; Dreier, et al., (2000) J Mol Biol 303:489-502; Liu, et al., (2002) J Biol Chem 277:3850-6), as well as those that recognize various CNN or TNN triplets (Dreier, et al., (2005) J Biol Chem 280:35588-97; Jamieson, et al., (2003) Nature Rev Drug Discov 2:361-8). See also, Durai, et al., (2005) Nucleic Acids Res 33:5978-90; Segal, (2002) Methods 26:76-83; Porteus and Carroll, (2005) Nat Biotechnol 23:967-73; Pabo, et al., (2001) Ann Rev Biochem 70:313-40; Wolfe, et al., (2000) Ann Rev Biophys Biomol Struct 29: 183-212; Segal and Barbas, (2001) Curr Opin Biotechnol 12:632-7; Segal, et al., (2003) Biochemistry 42:2137-48; Beerli and Barbas, (2002) Nat Biotechnol 20: 135-41; Carroll, et al., (2006) Nature Protocols 1:1329; Ordiz, et al., (2002) Proc Natl Acad Sci USA 99: 13290-5; Guan, et al., (2002) Proc Natl Acad Sci USA 99: 13296-301.

For more information on ZFNs and TALENs (as well as methods of their delivery), refer to Sanjana et al., Nat Protoc. 2012 Jan. 5; 7(1): 171-92 as well as international patent applications WO2002099084; WO00/42219, WO02/42459; WO2003062455; WO03/080809; WO05/014791; WO05/084190; WO08/021207; WO09/042186; WO09/054985; WO10/079430; and WO10/065123; U.S. Pat. Nos. 8,685,737; 6,140,466; 6,511,808; and 6,453,242; and US Patent Application Nos. 2011/0145940, 2003/0059767, and 2003/0108880; all of which are hereby incorporated by reference in their entirety.

In some cases (e.g., in the case of restriction enzymes), the recognition sequence is a constant (does not change) for the given protein (e.g., the recognition sequence for the BamHI restriction enzyme is G\hat{ }GATCC). In some cases, the sequence specific DNA endonuclease is ‘programmable’ in the sense that the protein (or its associated RNA in the case of CRISPR/Cas endonucleases) can be modified/engineered to recognize a desired recognition sequence. In some cases (e.g., in cases where the sequence specific DNA endonuclease is a meganuclease and/or in cases where the sequence specific DNA endonuclease is a CRISPR/Cas endonuclease), the recognition sequence has a length of 14 or more nucleotides (nt) (e.g., 15 or more, 16 or more, 17 or more, 18 or more, 19 or more, or 20 or more nt). In some cases, the recognition sequence has a length in a range of from 14-40 nt (e.g., 14-35, 14-30, 14-25, 15-40, 15-35, 15-30, 15-25, 16-40, 16-35, 16-30, 16-25, 17-40, 17-35, 17-30, or 17-25 nt). In some cases, the recognition sequence has a length of 14 or more base pairs (bp) (e.g., 15 or more, 16 or more, 17 or more, 18 or more, 19 or more, or 20 or more bp). In some cases, the recognition sequence has a length in a range of from 14-40 bp (e.g., 14-35, 14-30, 14-25, 15-40, 15-35, 15-30, 15-25, 16-40, 16-35, 16-30, 16-25, 17-40, 17-35, 17-30, or 17-25 bp).

When referring above to the lengths of a recognition sequence, the double-stranded helix and the recognition sequence can be thought of in terms of base pairs (bp), while in some cases (e.g., in the case of CRISPR/Cas endonucleases) the recognition sequence is recognized in single stranded form (e.g., a guide RNA of a CRISPR/Cas endonuclease can hybridize to the SARS-CoV-2 DNA) and the recognition sequence can be thought of in terms of nucleotides (nt). However, when using ‘bp’ or‘nt’ herein when referring to a recognition sequence, this terminology is not intended to be limiting. As an example, if a particular method or composition described herein encompasses both types of sequence specific DNA endonuclease (those that recognize ‘bp’ and those that recognize ‘nt’), either of the terms ‘nt’ or ‘bp’ can be used without limiting the scope of the sequence specific DNA endonuclease, because one of ordinary skill in the art would readily understand which term (‘nt’ or ‘bp’) would appropriately apply, and would understand that this depends on which protein is chosen. In the case of a length limitation of the recognition sequence, one of ordinary skill in the art would understand that the length limitation being discussed equally applies regardless of whether the term ‘nt’ or‘bp’ is used.

### Chew Back (Exonuclease Digestion)

After the circular SARS-CoV-2 DNAs are cleaved, generating a population of cleaved linear SARS-CoV-2 DNAs, the open ends of the linear SARS-CoV-2 DNAs are digested (chewed back) by exonucleases. Many different exonucleases will be known to one of ordinary skill in the art and any convenient exonuclease can be used. In some cases, a 5′ to 3′ exonuclease is used. In some cases, a 3′ to 5′ exonuclease is used. In some cases, an exonuclease is used that has both 5′ to 3′ and 3′ to 5′ exonuclease activity. In some cases, more than one exonuclease is used (e.g., 2 exonucleases). In some cases, the population of cleaved linear SARS-CoV-2 DNAs is contacted with a 5′ to 3′ exonuclease and a 3′ to 5′ exonuclease (e.g., simultaneously or one before the other).

In some cases, a T4 DNA polymerase is used as a 3′ to 5′ exonuclease (in the absence of dNTPs, T4 DNA polymerase has 3′ to 5′ exonuclease activity). In some cases, RecJ is used as a 5′ to 3′ exonuclease. In some cases, T4 DNA polymerase (in the absence of dNTPs) and RecJ are used. Examples of exonucleases include but are not limited to: DNA polymerase (e.g., T4 DNA polymerase) (in the absence of dNTPs), lambda exonuclease (5′->3′), T5 exonuclease (5′->3′), exonuclease III (3′->5′), exonuclease V (5′->3′ and 3′->5′), T7 exonuclease (5′->3′), exonuclease T, exonuclease VII (truncated) (5′->3′), and RecJ exonuclease (5′->3′).

The rate of DNA digestion (chew back) is sensitive to temperature, thus the size of the desired deletion can be controlled by regulating the temperature during exonuclease digestion. For example, in the examples section below when using T4 DNA polymerase (in the absence of dNTPs) and RecJ as the exonucleases, the double-end digestion rate (chew back rate) proceeded at a rate of 50 bp/min at 37° C. and at a reduced rate at lower temperatures (e.g., as discussed in the examples section below). Thus, temperature can be decreased or increased and/or digestion time can be decreased or increased to control the size of deletion (i.e., the amount of exonuclease digestion). For example, in some cases, the temperature and time are adjusted so that exonuclease digestion causes a deletion in a desired size range. As an illustrative example, if a deletion in a range of from 500-1000 base pairs (bp) is desired, the time and temperature of digestion can be adjusted so that 250-500 nucleotides are removed from each end of the linearized (cut) SARS-CoV-2 DNA, i.e., the size of the deletion is the sum of the number of nucleotides removed from each end of the linearized SARS-CoV-2 DNA. In some cases, the temperature and time are adjusted so that exonuclease digestion causes a deletion having a size in a range of from 20-1000 bp (e.g., from 20-50, 40-80, 20-100, 40-100, 20-200, 40-200, 60-100, 60-200, 80-150, 80-250, 100-250, 150-350, 100-500, 200-500, 200-700, 300-800, 400-800, 500-1000, 700-1000, 20-800, 50-1000, 100-1000, 250-1000, 50-1000, 50-750, 100-1000, or 100-750 bp).

In some cases, contacting with an exonuclease (one or more exonucleases) is performed at a temperature in a range of from room temperature (e.g., 25° C.) to 40° C. (e.g., from 25-37° C., 30-37° C., 32-40° C., or 30-40° C.). In some cases, contacting with an exonuclease is performed at 37° C. In some cases, contacting with an exonuclease is performed at 32° C. In some cases, contacting with an exonuclease is performed at 30° C. In some cases, contacting with an exonuclease is performed at 25° C. In some cases, contacting with an exonuclease is performed at room temperature.

In some cases, the SARS-CoV-2 DNA is contacted with an exonuclease (one or more exonucleases) for a period of time in a range of from 10 seconds to 40 minutes (e.g., from 10 seconds to 30 minutes, 10 seconds to 20 minutes, 10 seconds to 15 minutes, 10 seconds to 10 minutes, 30 seconds to 30 minutes, 30 seconds to 20 minutes, 30 seconds to 15 minutes, 30 seconds to 12 minutes, 30 seconds to 10 minutes, 1 to 40 minutes, 1 to 30 minutes, 1 to 20 minutes, 1 to 15 minutes, 1 to 10 minutes, 3 to 40 minutes, 3 to 30 minutes, 3 to 20 minutes, 3 to 15 minutes, 3 to 12 minutes, or 3 to 10 minutes). In some cases, the contacting is for a period of time in a range of from 20 seconds to 15 minutes.

After DNA digestion (chew back), the remaining overhanging DNA ends can be repaired (e.g., using T4 DNA Polymerase plus dNTPs) or in some cases the single stranded overhangs can be removed (e.g., using a nuclease such as mung bean nuclease that cleaves single stranded DNA but not double stranded DNA). For example, if only a 5′ to 3′ or 3′ to 5′ exonuclease is used, a nuclease specific for single stranded DNA (i.e., that does not cut double stranded DNA) (e.g., mung bean nuclease) can be used to remove the overhang.

The step of contacting with one or more exonucleases (i.e., chew back) can be carried out in the presence or absence of a single strand binding protein (SSB protein). An SSB is a protein that binds to exposed single stranded DNA ends, which can achieve numerous results, including but not limited to: (i) helping stabilize the DNA by preventing nucleases from accessing the DNA, and (ii) preventing hairpin formation within the single stranded DNA. Examples of SSB proteins include but are not limited to a eukaryotic SSB protein (e.g., replication protein A (RPA)); bacterial SSB protein; and viral SSB proteins. In some cases, the step of contacting with one or more exonucleases is performed in the presence of an SSB. In some cases, the step of contacting with one or more exonucleases is performed in the absence of an SSB.

### Barcode

In some embodiments, the members of a library are ‘tagged’ by adding a barcode to the SARS-CoV-2 DNAs after exonuclease digestion (and after remaining overhanging DNA ends are repaired/removed). The addition of a barcode can be performed prior to or simultaneously with re-circularizing (ligation). As used herein, term “barcode” is used to mean a stretch of nucleotides having a sequence that uniquely tags members of the library for future identification. For example, in some cases, a barcode cassette (from a pool of random barcode cassettes) can be added and the library sequenced so that it is known which barcode sequence is associated with which particular member, i.e., with which particular deletion (e.g., a lookup table can be created such that each member of a deletion library has a unique barcode). In this way, members of a deletion library can be tracked and accounted for by virtue of presence of the barcode (instead of having to identify the members by determining the location of deletion). Identifying the presence of a short stretch of nucleotides using any convenient assay can easily be accomplished. Use of such barcodes is easier than isolating and sequencing individual members (in order to determine location of deletion) each time the library is used for a given experiment. For example, one can readily determine which library members are present before an experiment (e.g., before introducing library members into cells to assay for viral infectivity), and compare this to which members are present after the experiment by simply assaying for the presence of the barcode before and after, e.g., using high throughput sequencing, a microarray, PCR, qPCR, or any other method that can detect the presence/absence of a barcode sequence.

In some cases, a barcode is added as a cassette. A barcode cassette is a stretch of nucleotides that have at least one constant region (a region shared by all members receiving the cassette) and a barcode region (i.e., a barcode sequence—a region unique to the members that receive the barcode such that the barcode uniquely marks the members of the library). For example, a barcode cassette can include (i) a constant region that is a primer site, which site is in common among the barcode cassettes used, and (ii) a barcode sequence that is a unique tag, e.g., can be a stretch of random sequence. In some cases, a barcode cassette includes a barcode region flanked by two constant regions (e.g., two different primer sites). As an illustrative example, in some cases a barcode cassette is a 60 bp cassette that includes a 20 bp random barcode flanked by 20 bp primer binding sites (e.g., see FIG. 4).

A barcode sequence can have any convenient length and is preferably long enough so that it uniquely marks the members of a given library of interest. In some cases, the barcode sequence has a length of from 15 bp to 40 bp (e.g., from 15-35 bp, 15-30 bp, 15-25 bp, 17-40 bp, 17-35 bp, 17-30 bp, or 17-25 bp). In some cases, the barcode sequence has a length of 20 bp. Likewise, a barcode cassette can have any convenient length, and this length depends on the length of the barcode sequence plus the length of the constant region(s). In some cases, the barcode cassette has a length of from 40 bp to 100 bp (e.g., from 40-80 bp, 45-100 bp, 45-80 bp, 45-70 bp, 50-100 bp, 50-80 bp, or 50-70 bp). In some cases, the barcode cassette has a length of 60 bp.

A barcode or barcode cassette can be added using any convenient method. For example, a linear SARS-CoV-2 DNA can be recircularized by ligation to a 3′-dT-tailed barcode cassette drawn from a pool of random barcode cassettes. The nicked hemiligation product can then be sealed and transformed into a host cell, e.g., a bacterial cell.

### Generating a Product

In some cases, a subject method includes a step of generating (e.g., from a generated library of circularized SARS-CoV-2 deletion DNAs) at least one of: linear double stranded DNA (dsDNA) products (e.g., via cleavage of the circular DNA, via PCR, etc.), linear single stranded DNA (ssDNA) products (e.g., via transcription and reverse transcription), linear single stranded RNA (ssRNA) products (e.g., via transcription), and linear double stranded RNA (dsRNA) products. If so desired, the linear SARS-CoV-2 products can then be introduced into a cell (e.g., mammalian cell). For example, a common technique for RNA viruses is to perform in vitro transcription from a dsDNA template (circular or linear) to make RNA, and then to introduce this RNA into cells (e.g., via electroporation, chemical methods, etc.) to generate viral stocks.

Also, within the scope of the disclosure are kits. For example, in some cases a subject kit can include one or more of (in any combination): (i) a population of circular SARS-CoV-2 DNAs as described herein, (ii) a transposon cassette as described herein, (iii) a sequence specific DNA endonuclease as described herein, (iv) one or more guide RNAs for a CRISPR/Cas endonuclease as described herein, (v) a population of barcodes and/or barcode cassettes as described herein, and (vi) a population of host cells, e.g., for propagation of the library, for assaying for viral infectivity, etc., as described herein. In some cases, a subject kit can include instructions for use. Kits typically include a label indicating the intended use of the contents of the kit. The term label includes any writing, or recorded material supplied on or with the kit, or which otherwise accompanies the kit.

### SARS-CoV-2 Virus

The SARS-CoV-2 virus has a single-stranded RNA genome with about 29891 nucleotides, that encode about 9860 amino acids. A SARS-CoV-2 selected RNA genome can be copied and made into a DNA by reverse transcription and formation of a cDNA. A linear SARS-CoV-2 DNA can be circularized by ligation of SARS-CoV-2 DNA ends.

A DNA sequence for the SARS-CoV-2 genome, with coding regions, is available as accession number NC_045512.2 from the NCBI website (provided as SEQ ID NO:1 herein).

```
        1 ATTAAAGGTT TATACCTTCC CAGGTAACAA ACCAACCAAC
      41 TTTCGATCTC TTGTAGATCT GTTCTCTAAA CGAACTTTAA
      81 AATCTGTGTG GCTGTCACTC GGCTGCATGC TTAGTGCACT
    121 CACGCAGTAT AATTAATAAC TAATTACTGT CGTTGACAGG
    161 ACACGAGTAA CTCGTCTATC TTCTGCAGGC TGCTTACGGT
    201 TTCGTCCGTG TTGCAGCCGA TCATCAGCAC ATCTAGGTTT
    241 CGTCCGGGTG TGACCGAAAG GTAAGATGGA GAGCCTTGTC
    281 CCTGGTTTCA ACGAGAAAAC ACACGTCCAA CTCAGTTTGC
    321 CTGTTTTACA GGTTCGCGAC GTGCTCGTAC GTGGCTTTGG
    361 AGACTCCGTG GAGGAGGTCT TATCAGAGGC ACGTCAACAT
    401 CTTAAAGATG GCACTTGTGG CTTAGTAGAA GTTGAAAAAG
    441 SCGTTTTGCC TCAACTTGAA CAGCCCTATG TGTTCATCAA
    481 ACGTTCGGAT GCTCGAACTG CACCTCATGG TCATGTTATG
    521 GTTGAGCTGG TAGCAGAACT CGAAGGCATT CAGTACGGTC
    561 GTAGTGGTGA GAGACTTGGT GTCCTTGTCC CTCATGTGGG
    601 CGAAATACCA GTGGCTTACC GCAAGGTTCT TCTTCGTAAG
    641 AACGGTAATA AAGGAGCTGG TGGCCATAGT TACGGCGCCG
    681 ATCTAAAGTC ATTTGACTTA GGCGACGAGC TTGGCACTGA
    721 TCCTTATGAA GATTTTCAAG AAAACTGGAA CACTAAACAT
    761 AGCAGTGGTG TTACCCGTGA ACTCATGCGT GAGCTTAACG
    801 GAGGGGCATA CACTCGCTAT GTCGATAACA ACTTCTGTGG
    841 CCCTGATGGC TACCCTCTTG AGTGCATTAA AGACCTTCTA
    881 GCACGTGCTG GTAAAGCTTC ATGCACTTTG TCCGAACAAC
    921 TGGACTTTAT TGACACTAAG AGGGGTGTAT ACTGCTGCCG
    961 TGAACATGAG CATGAAATTG CTTGGTACAC GGAACGTTCT
  1001 GAAAAGAGCT ATGAATTGCA GACACCTTTT GAAATTAAAT
  1041 TGGCAAAGAA ATTTGACACC TTCAATGGGG AATGTCCAAA
  1081 TTTTGTATTT CCCTTAAATT CCATAATCAA GACTATTCAA
  1121 CCAAGGGTTG AAAAGAAAAA GCTTGATGGC TTTATGGGTA
  1161 GAATTCGATC TGTCTATCCA GTTGCGTCAC CAAATGAATG
  1201 CAACCAAATG TGCCTTTCAA CTCTCATGAA GTGTGATCAT
  1241 TGTGGTGAAA CTTCATGGCA GACGGGCGAT TTTGTTAAAG
  1281 CCACTTGCGA ATTTTGTGGC ACTGAGAATT TGACTAAAGA
  1321 AGGTGCCACT ACTTGTGGTT ACTTACCCCA AAATGCTGTT
  1361 GTTAAAATTT ATTGTCCAGC ATGTCACAAT TCAGAAGTAG
  1401 GACCTGAGCA TAGTCTTGCC GAATACCATA ATGAATCTGG
  1441 CTTGAAAACC ATTCTTCGTA AGGGTGGTCG CACTATTGCC
  1481 TTTGGAGGCT GTGTGTTCTC TTATGTTGGT TGCCATAACA
  1521 AGTGTGCCTA TTGGGTTCCA CGTGCTAGCG CTAACATAGG
  1561 TTGTAACCAT ACAGGTGTTG TTGGAGAAGG TTCCGAAGGT
  1601 CTTAATGACA ACCTTCTTGA AATACTCCAA AAAGAGAAAG
  1641 TCAACATCAA TATTGTTGGT GACTTTAAAC TTAATGAAGA
  1681 GATCGCCATT ATTTTGGCAT CTTTTTCTGC TTCCACAAGT
  1721 GCTTTTGTGG AAACTGTGAA AGGTTTGGAT TATAAAGCAT
  1761 TCAAACAAAT TGTTGAATCC TGTGGTAATT TTAAAGTTAC
  1801 AAAAGGAAAA GCTAAAAAAG GTGCCTGGAA TATTGGTGAA
  1841 CAGAAATCAA TACTGAGTCC TCTTTATGCA TTTGCATCAG
  1881 AGGCTGCTCG TGTTGTACGA TCAATTTTCT CCCGCACTCT
  1921 TGAAACTGCT CAAAATTCTG TGCGTGTTTT ACAGAAGGCC
  1961 GCTATAACAA TACTAGATGG AATTTCACAG TATTCACTGA
  2001 GAGTCATTGA TGCTATGATG TTCACATCTG ATTTGGCTAC
  2041 TAACAATCTA GTTGTAATGG CCTACATTAC AGGTGGTGTT
  2081 GTTCAGTTGA CTTCGCAGTG GCTAACTAAC ATCTTTGGCA
  2121 CTGTTTATGA AAAACTCAAA CCCGTCCTTG ATTGGCTTGA
  2161 AGAGAAGTTT AAGGAAGGTG TAGAGTTTCT TAGAGACGGT
  2201 TGGGAAATTG TTAAATTTAT CTCAACCTGT GCTTGTGAAA
  2241 TTGTCGGTGG ACAAATTGTC ACCTGTGCAA AGGAAATTAA
  2281 GGAGAGTGTT CAGACATTCT TTAAGCTTGT AAATAAATTT
  2321 TTGGCTTTGT GTGCTGACTC TATCATTATT GGTGGAGCTA
  2361 AACTTAAAGC CTTGAATTTA GGTGAAACAT TTGTCACGCA
  2401 CTCAAAGGGA TTGTACAGAA AGTGTGTTAA ATCCAGAGAA
  2441 GAAACTGGCC TACTCATGCC TCTAAAAGCC CCAAAAGAAA
  2481 TTATCTTCTT AGAGGGAGAA ACACTTCCCA CAGAAGTGTT
  2521 AACAGAGGAA GTTGTCTTGA AAACTGGTGA TTTACAACCA
  2561 TTAGAACAAC CTACTAGTGA AGCTGTTGAA GCTCCATTGG
  2601 TTGGTACACC AGTTTGTATT AACGGGCTTA TGTTGCTCGA
  2641 AATCAAAGAC ACAGAAAAGT ACTGTGCCCT TGCACCTAAT
  2681 ATGATGGTAA CAAACAATAC CTTCACACTC AAAGGCGGTG
  2721 CACCAACAAA GGTTACTTTT GGTGATGACA CTGTGATAGA
  2761 AGTGLAAGGT TACAAGAGTG TGAATATCAC TTTTGAACTT
  2801 GATGAAAGGA TTGATAAAGT ACTTAATGAG AAGTGCTCTG
  2841 CCTATACAGT TGAACTCGGT ACAGAAGTAA ATGAGTTCGC
  2881 CTGTGTTGTG GCAGATGCTG TCATAAAAAC TTTGCAACCA
  2921 GTATCTGAAT TACTTACACC ACTGGGCATT GATTTAGATG
  2961 AGTGGAGTAT GGCTACATAC TACTTATTTG ATGAGTCTGG
  3001 TGAGTTTAAA TTGGCTTCAC ATATGTATTG TTCTTTCTAC
  3041 CCTCCAGATG AGGATGAAGA AGAAGGTGAT TGTGAAGAAG
  3081 AAGAGTTTGA GCCATCAACT CAATATGAGT ATGGTACTGA
  3121 AGATGATTAC CAAGGTAAAC CTTTGGAATT TGGTGCCACT
  3161 TCTGCTGCTC TTCAACCTGA AGAAGAGCAA GAAGAAGATT
  3201 GGTTAGATGA TGATAGTCAA CAAACTGTTG GTCAACAAGA
  3241 CGGCAGTGAG GACAATCAGA CAACTACTAT TCAAACAATT
  3281 GTTGAGGTTC AACCTCAATT AGAGATGGAA CTTACACCAG
  3321 TTGTTCAGAC TATTGAAGTG AATAGTTTTA GTGGTTATTT
  3361 AAAACTTACT GACAATGTAT ACATTAAAAA TGCAGACATT
  3401 GTGGAAGAAG CTAAAAAGGT AAAACCAACA GTGGTTGTTA
  3441 ATGCAGCCAA TGTTTACCTT AAACATGGAG GAGGTGTTGC
  3481 AGGAGCCTTA AATAAGGCTA CTAACAATGC CATGCAAGTT
  3521 GAATCTGATG ATTACATAGC TACTAATGGA CCACTTAAAG
  3561 TGGGTGGTAG TTGTGTTTTA AGCGGACACA ATCTTGCTAA
  3601 ACACTGTCTT CATGTTGTCG GCCCAAATGT TAACAAAGGT
  3641 GAAGACATTC AACTTCTTAA GAGTGCTTAT GAAAATTTTA
  3681 ATCAGCACGA AGTTCTACTT GCACCATTAT TATCAGCTGG
  3721 TATTTTTGGT GCTGACCCTA TACATTCTTT AAGAGTTTGT
  3761 GTAGATACTG TTCGCACAAA TGTCTACTTA GCTGTCTTTG
  3801 ATAAAAATCT CTATGACAAA CTTGTTTCAA GCTTTTTGGA
  3841 AATGAAGAGT GAAAAGCAAG TTGAACAAAA GATCGCTGAG
  3881 ATTCCTAAAG AGGAAGTTAA GCCATTTATA ACTGAAAGTA
  3921 AACCTTCAGT TGAACAGAGA AAACAAGATG ATAAGAAAAT
  3961 CAAAGCTTGT GTTGAAGAAG TTACAACAAC TCTGGAAGAA
  4001 ACTAAGTTCC TCACAGAAAA CTTGTTACTT TATATTGACA
  4041 TTAATGGCAA TCTTCATCCA GATTCTGCCA CTCTTGTTAG
  4081 TGACATTGAC ATCACTTTCT TAAAGAAAGA TGCTCCATAT
  4121 ATAGTGGGTG ATGTTGTTCA AGAGGGTGTT TTAACTGCTG
  4161 TGGTTATACC TACTAAAAAG GCTGGTGGCA CTACTGAAAT
  4201 GCTAGCGAAA GCTTTGAGAA AAGTGCCAAC AGACAATTAT
  4241 ATAACCACTT ACCCGGGTCA GGGTTTAAAT GGTTACACTG
  4281 TAGAGGAGEC AAAGACAGTG CTTAAAAAGT GTAAAAGTGC
  4321 CTTTTACATT CTACCATCTA TTATCTCTAA TGAGAAGCAA
  4361 GAAATTCTTG GAACTGTTTC TTGGAATTTG CGAGAAATGC
  4401 TTGCACATGC AGAAGAAACA CGCAAATTAA TGCCTGTCTG
  4441 TGTGGAAACT AAAGCCATAG TTTCAACTAT ACAGCGTAAA
  4481 TATAAGGGTA TTAAAATACA AGAGGGTGTG GTTGATTATG
  4521 GTGCTAGATT TTACTTTTAC ACCAGTAAAA CAACTGTAGC
  4561 GTCACTTATC AACACACTTA ACGATCTAAA TGAAACTCTT
  4601 GTTACAATGC CACTTGGCTA TGTAACACAT GGCTTAAATT
  4641 TGGAAGAAGC TGCTCGGTAT ATGAGATCTC TCAAAGTGCC
  4681 AGCTACAGTT TCTGTTTCTT CACCTGATGC TGTTACAGCG
  4721 TATAATGGTT ATCTTACTTC TTCTTCTAAA ACACCTGAAG
  4761 AACATTTTAT TGAAACCATC TCACTTGCTG GTTCCTATAA
  4801 AGATTGGTCC TATTCTGGAC AATCTACACA ACTAGGTATA
  4841 GAATTTCTTA AGAGAGGTGA TAAAAGTGTA TATTACACTA
  4881 GTAATCCTAC CACATTCCAC CTAGATGGTG AAGTTATCAC
  4921 CTTTGACAAT CTTAAGACAC TTCTTTCTTT GAGAGAAGTG
  4961 AGGAGTATTA AGGTGTTTAG AACAGTAGAC AACATTAACC
  5001 TCCACACGCA AGTTGTGGAC ATGTCAATGA CATATGGACA
  5041 AGAGTTTGGT CCAACTTATT TGGATGGAGC TGATGTTACT
  5081 AAAATAAAAC CTCATAATTC AGATGAAGGT AAAACATTTT
  5121 ATGTTTTAGC TAATGATGAG ACTCTACGTG TTGAGGCTTT
  5161 TGAGTAGTAC CACACAACTG ATCCTAGTTT TCTGGGTAGG
  5201 TACATGTCAG CATTAAATCA CACTAAAAAG TGGAAATACC
  5241 CACAAGTTAA TGGTTTAACT TCTATTAAAT GGGCAGATAA
  5281 CAACTGTTAT CTTGCCACTG CATTGTTAAC ACTCCAACAA
  5321 ATAGAGTTGA AGTTTAATCC ACCTGCTCTA CAAGATGCTT
  5361 ATTACAGAGC AAGGGCTGGT GAAGCTGCTA ACTTTTGTGC
  5401 ACTTATCTTA GCCTACTGTA ATAAGACAGT AGGTGAGTTA
  5441 GGTGATGTTA GAGAAACAAT GAGTTACTTG TTTCAACATG
  5481 CCAATTTAGA TTCTTGCAAA AGAGTCTTGA ACGTGGTGTG
  5521 TAAAACTTGT GGACAACAGC AGACAACCCT TAAGGGTGTA
  5561 GAAGCTGTTA TGTACATGGG CACACTTTCT TATGAACAAT
  5601 TTAAGAAAGG TGTTCAGATA CCTTGTACGT GTGGTAAACA
  5641 AGCTACAAAA TATCTAGTAG AACAGGAGTC ACCTTTTGTT
  5681 ATGATGTCAG CACCACCTGC TCAGTATGAA CTTAAGCATG
  5721 GTACATTTAC TTGTGCTAGT GAGTACACTG GTAATTACCA
  5761 GTGTGGTCAC TATAAACATA TAACTTCTAA AGAAACTTTG
  5801 TATTGCATAG ACGGTGCTTT ACTTACAAAG TCCTCAGAAT
  5841 ACAAAGGTCC TATTACGGAT GTTTTCTACA AAGAAAACAG
  5881 TTACACAACA ACCATAAAAC CAGTTACTTA TAAATTGGAT
  5921 GGTGTTGTTT GTACAGAAAT TGACCCTAAG TTGGACAATT
  5961 ATTATAAGAA AGACAATTCT TATTTCACAG AGCAACCAAT
  6001 TGATCTTGTA CCAAACCAAC CATATCCAAA CGCAAGCTTC
  6041 GATAATTTTA AGTTTGTATG TGATAATATC AAATTTGCTG
  6081 ATGATTTAAA CCAGTTAACT GGTTATAAGA AACCTGCTTC
  6121 AAGAGAGCTT AAAGTTACAT TTTTCCCTGA CTTAAATGGT
  6161 GATGTGGTGG CTATTGATTA TAAACACTAG ACACCCTCTT
  6201 TTAAGAAAGG AGCTAAATTG TTACATAAAC CTATTGTTTG
  6241 GCATGTTAAC AATGCAACTA ATAAAGCCAC GTATAAACCA
  6281 AATACCTGGT GTATACGTTG TCTTTGGAGC ACAAAACGAG
  6321 TTGAAACATC AAATTCGTTT GATGTACTGA AGTCAGAGGA
  6361 CGCGCAGGGA ATGGATAATC TTGCCTGCGA AGATCTAAAA
  6401 CCAGTCTCTG AAGAAGTAGT GGAAAATCCT ACCATACAGA
  6441 AAGACGTTCT TGAGTGTAAT GTGAAAACTA CCGAAGTTGT
  6481 AGGAGALATT ATACTTAAAC CAGCAAATAA TAGTTTAAAA
  6521 ATTACAGAAG AGGTTGGCCA CACAGATCTA ATGGCTGCTT
  6561 ATGTAGACAA TTCTAGTCTT ACTATTAAGA AACCTAATGA
  6601 ATTATCTAGA GTATTAGGTT TGAAAACCCT TGCTACTCAT
  6641 GGTTTAGCTG CTGTTAATAG TGTCCCTTGG GATACTATAG
  6681 CTAATTATGC TAAGCCTTTT CTTAACAAAG TTGTTAGTAC
  6721 AACTACTAAC ATAGTTACAC GGTGTTTAAA CCGTGTTTGT
  6761 ACTAATTATA TGCCTTATTT CTTTACTTTA TTGCTACAAT
  6801 TGTGTACTTT TACTAGAAGT ACAAATTCTA GAATTAAAGC
  6841 ATCTATGCCG ACTACTATAG CAAAGAATAC TGTTAAGAGT
  6881 GTCGGTAAAT TTTGTCTAGA GGCTTCATTT AATTATTTGA
  6921 AGTCACCTAA TTTTTCTAAA CTGATAAATA TTATAATTTG
  6961 GTTTTTACTA TTAAGTGTTT GCCTAGGTTC TTTAATCTAC
  7001 TCAACCGCTG CTTTAGGTGT TTTAATGTCT AATTTAGGCA
  7041 TGCCTTCTTA CTGTACTGGT TACAGAGAAG GCTATTTGAA
  7081 CTCTACTAAT GTCACTATTG CAACCTACTG TACTGGTTCT
  7121 ATACCTTGTA GTGTTTGTCT TAGTGGTTTA GATTCTTTAG
  7161 ACACCTATCC TTCTTTAGAA ACTATACAAA TTACCATTTC
  7201 ATCTTTTAAA TGGGATTTAA CTGCTTTTGG CTTAGTRGCA
  7241 GAGTGGTTTT TGGCATATAT TCTTTTCACT AGGTTTTTCT
  7281 ATGTACTTGG ATTGGCTGCA ATCATGCAAT TGTTTTTCAG
  7321 CTATTTTGCA GTACATTTTA TTAGTAATTC TTGGCTTATG
  7361 TGGTTAATAA TTAATCTTGT ACAAATGGCC CCGATTTCAG
  7401 CTATGGTTAG AATGTACATC TTCTTTGCAT CATTTTATTA
  7441 TGTATGGAAA AGTTATGTGC ATGTTGTAGA CGGTTGTAAT
  7481 TCATCAACTT GTATGATGTG TTACAAACGT AATAGAGCAA
  7521 CAAGAGTCGA ATGTACAACT ATTGTTAATG GTGTTAGAAG
  7561 GTCCTTTTAT GTCTATGCTA ATGGAGGTAA AGGCTTTTGC
  7601 AAACTACACA ATTGGAATTG TGTTAATTGT GATACATTCT
  7641 GTGCTGGTAG TACATTTATT AGTGATGAAG TTGCGAGAGA
  7681 CTTGTCACTA CAGTTTAAAA GACCAATAAA TCCTACTGAC
  7721 CAGTCTTCTT ACATCGTTGA TAGTGTTACA GTGAAGAATG
  7761 GTTCCATCCA TCTTTACTTT GATAAAGCTG GTCAAAAGAC
  7801 TTATGAAAGA CATTCTCTCT CTCATTTTGT TAACTTAGAC
  7841 AACCTGAGAG CTAATAACAC TAAAGGTTCA TTGCCTATTA
  7881 ATGTTATAGT TTTTGATGGT AAATCAAAAT GTGAAGAATC
  7921 ATCTGCAAAA TCAGCGTCTG TTTACTACAG TCAGCTTATG
  7961 TGTCAACCTA TACTGTTACT AGATCAGGCA TTAGTGTCTG
  8001 ATGTTGGTGA TAGTGCGGAA GTTGCAGTTA AAATGTTTGA
  8041 TGCTTACGTT AATACGTTTT CATCAACTTT TAACGTACCA
  8081 ATGGAAAAAC TCAAAACACT AGTTGCAACT GCAGAAGCTG
  8121 AACTTGCAAA GAATGTGTCC TTAGACAATG TCTTATCTAC
  8161 TTTTATTTCA GCAGCTCGGC AAGGGTTTGT TGATTCAGAT
  8201 GTAGAAACTA AAGATGTTGT TGAATGTCTT AAATTGTCAC
  8241 ATCAATCTGA CATAGAAGTT ACTGGCGATA GTTGTAATAA
  8281 CTATATGCTC ACCTATAACA AAGTTGAAAA CATGACACCC
  8321 CGTGACCTTG GTGCTTGTAT TGACTGTAGT GCGCGTCATA
  8361 TTAATGCGCA GGTAGCAAAA AGTCACAACA TTGCTTTGAT
  8401 ATGGAACGTT AAAGATTTCA TGTCATTGTC TGAACAACTA
  8441 CGAAAACAAA TACGTAGTGC TGCTAAAAAG AATAACTTAC
  8481 CTTTTAAGTT GACATGTGCA ACTACTAGAC AAGTTGTTAA
  8321 TGTTGTAACA ACAAAGATAG CACTTAAGGG TGGTAAAATT
  8561 GTTAATAATT GGTTGAAGCA GTTAATTAAA GTTACACTTG
  8601 TGTTCCTTTT TGTTGCTGCT ATTTTCTATT TAATAACACC
  8641 TGTTCATGTC ATGTCTAAAC ATACTGACTT TTCAAGTGAA
  8681 ATCATAGGAT ACAAGGCTAT TGATGGTGGT GTCACTCGTG
  8721 ACATAGCATC TACAGATACT TGTTTTGCTA ACAAACATGC
  8761 TGATTTTGAC ACATGGTTTA GCCAGCGTGG TGGTAGTTAT
  8801 ACTAATGACA AAGCTTGCCC ATTGATTGCT GCAGTCATAA
  8841 CAAGAGAAGT GGGTTTTGTC GTGCCTGGTT TGCCTGGCAC
  8881 GATATTACGC ACAACTAATG GTGACTTTTT GCATTTCTTA
  8921 CCTAGAGTTT TTAGTGCAGT TGGTAACATC TGTTACACAC
  8961 CATCAAAACT TATAGAGTAC ACTGACTTTG CAACATCAGC
  9001 TTGTGTTTTG GCTGCTGAAT GTACAATTTT TAAAGATGCT
  9041 TCTGGTAAGC CAGTACCATA TTGTTATGAT ACCAATGTAC
  9081 TAGAAGGTTC TGTTGCTTAT GAAAGTTTAC GCCCTGACAC
  9121 ACGTTATGTG CTCATGGATG GCTCTATTAT TCAATTTCCT
  9161 AACACCTACC TTGAAGGTTC TGTTAGAGTG GTAACAACTT
  9201 TTGATTCTGA GTACTGTAGG CACGGCACTT GTGAAAGATC
  9241 AGAAGCTGGT GTTTGTGTAT CTACTAGTGG TAGATGGGTA
  9281 CTTAACAATG ATTATTACAG ATCTTTACCA GGAGTTTTCT
  9321 GTGGTGTAGA TGCTGTAAAT TTACTTACTA ATATGTTTAC
  9361 ACCACTAATT CAACCTATTG GTGCTTTGGA CATATCAGCA
  9401 TCTATAGTAG CTGGTGGTAT TGTAGCTATC GTAGTAACAT
  9441 GCCTTGCCTA CTATTTTATG AGGTTTAGAA GAGCTTTTGG
  9481 TGAATACAGT CATGTAGTTG CCTTTAATAC TTTACTATTC
  9521 CTTATGTCAT TCACTGTACT CTGTTTAACA CCAGTTTACT
  9561 cattcttacc TGGTGTTTAT TCTGTTATTT ACTTGTACTT
  9601 GACATTTTAT CTTACTAATG ATGTTTCTTT TTTAGCACAT
  9641 ATTCAGTGGA TGGTTATGTT CACACCTTTA GTACCTTTCT
  9681 GGATAACAAT TGCTTATATC ATTTGTATTT CCACAAAGCA
  9721 TTTCTATTGG TTOTTTAGTA ATTAGGTAAA GAGACGTGTA
  9761 GTCTTTAATG GTGTTTCCTT TAGTACTTTT GAAGAAGCTG
  9801 CGCTGTGCAC CTTTTTGTTA AATAAAGAAA TGTATCTAAA
  9841 GTTGCGTAGT GATGTGCTAT TACCTCTTAC GCAATATAAT
  9881 AGATACTTAG CTCTTTATAA TAAGTACAAG TATTTTAGTG
  9921 GAGCAATGGA TACAACTAGC TACAGAGAAG CTGCTTGTTG
  9961 TCATCTCGCA AAGGCTCTCA ATGACTTCAG TAACTCAGGT
10001 TCTGATGTTC TTTACCAACC ACCACAAACC TCTATCACCT
10041 CAGCTGTTTT GCAGAGTGGT TTTAGAAAAA TGGCATTCCC
10081 ATCTGGTAAA GTTGAGGGTT GTATGGTACA AGTAACTTGT
10121 GGTACAACTA CACTTAACGG TCTTTGGCTT GATGAGGTAG
10161 TTTACTGTCC AAGACATGTG ATCTGCACCT CTGAAGACAT
10201 GCTTAACCCT AATTATGAAG ATTTACTCAT TCGTAAGTCT
10241 AATCATAATT TCTTGGTACA GGCTGGTAAT GTTCAACTCA
10281 GGGTTATTGG ACATTCTATG CAAAATTGTG TACTTAAGCT
10321 TAAGGTTGAT ACAGCCAATC CTAAGACACC TAAGTATAAG
10361 TTTGTTCGCA TTCAACCAGG ACAGACTTTT TCAGTGTTAG
10401 CTTGTTACAA TGGTTCACCA TCTGGTGTTT ACCAATGTGC
10441 TATGAGGCCC AATTTCACTA TTAAGGGTTC ATTCCTTAAT
10481 GGTTCATGTG GTAGTGTTGG TTTTAACATA GATTATGACT
10521 GTGTCTCTTT TTGTTACATG CACCATATGG AATTACCAAC
10561 TGGAGTTCAT GCTGGCACAG ACTTAGAAGG TAACTTTTAT
10601 GGACCTTTTG TTGACAGGCA AACAGCACAA GCAGCTGGTA
10641 CGGACACAAC TATTAGAGTT AATGTTTTAG CTTGGTTGTA
10681 CGCTGCTGTT ATAAATGGAG ACAGGTGGTT TCTCAATCGA
10721 TTTACCACAA CTCTTAATGA CTTTAACCTT GTGGCTATGA
10761 AGTACAATTA TGAACCTCTA ACACAAGACC ATGTTGACAT
10801 ACTAGGACCT CTTTCTGCTC AAACTGGAAT TGCCGTTTTA
10841 GATATGTGTG CTTCATTAAA AGAATTACTG CAAAATGGTA
10881 TGAATGGACG TACCATATTG GGTAGTGCTT TATTAGAAGA
10921 TGAATTTACA CCTTTTGATG TTGTTAGAGA ATGCTCAGGT
10961 GTTACTTTCC AAAGTGCAGT GAAAAGAACA ATCAAGGGTA
11001 CACACCACTG GTTGTTACTC ACAATTTTGA CTTCACTTTT
11041 AGTTTTAGTC CAGAGTACTC AATGGTCTTT GTTCTTTTTT
11081 TTGTATGAAA ATGCCTTTTT ACCTTTTGCT ATGGGTATTA
11121 TTGCTATGTC TGCTTTTGCA ATGATGTTTG TCAAACATAA
11161 GCATGCATTT CTCTGTTTGT TTTTGTTACC TTCTCTTGCC
11201 ACTGTAGCTT ATTTTAATAT GGTCTATATG CCTGCTAGTT
11241 GGGTGATGCG TATTATGACA TGGTTGGATA TGGTTGATAC
11281 TAGTTTGTCT GGTTTTAAGC TAAAAGACTG TGTTATGTAT
11321 GCATCAGCTG TAGTGTTACT AATCCTTATG ACAGCAAGAA
11361 CTGTGTATGA TGATGGTGCT AGGAGAGTGT GGACACTTAT
11401 GAATGTCTTG ACACTCGTTT ATAAAGTTTA TTATGGTAAT
11441 GCTTTAGATC AAGCCATTTC CATGTGGGCT CTTATAATCT
11481 CTGTTACTTC TAACTACTCA GGTGTAGTTA CAACTGTCAT
11521 GTTTTTGGCC AGAGGTATTG TTTTTATGTG TGTTGAGTAT
11561 TGCCCTATTT TCTTCATAAC TGGTAATACA CTTCAGTGTA
11601 TAATGCTAGT TTATTGTTTC TTAGGCTATT TTTGTACTTG
11641 TTACTTTGGC CTCTTTTGTT TACTCAACCG CTACTTTAGA
11681 CTGACTCTTG GTGTTTATGA TTACTTAGTT TCTACACAGG
11721 AGTTTAGATA TATGAATTCA CAGGGACTAG TCCCACCCAA
11761 GAATAGCATA GATGCCTTCA AACTCAACAT TAAATTGTTG
11801 GGTGTTGGTG GCAAACCTTG TATCAAAGTA GCCACTGTAC
11841 AGTCTAAAAT GTCAGATGTA AAGTGCACAT CAGTAGTCTT
11881 ACTCTCAGTT TTGCAACAAC TCAGAGTAGA ATCATCATCT
11921 AAATTGTGGG CTCAATGTGT CCAGTTACAC AATGACATTC
11961 TCTTAGCTAA AGATACTACT GAAGCCTTTG AAAAAATGGT
12001 TTCACTACTT TCTGTTTTGC TTTCCATGCA GGGTGCTGTA
12041 GACATAAACA AGCTTTGTGA AGAAATGCTG GACAACAGGG
12081 CAACCTTACA AGCTATAGCC TCAGAGTTTA GTTCCCTTCC
12121 ATCATATGCA GCTTTTGCTA CTGCTCAAGA AGCTTATGAG
12161 CAGGCTGTTG CTAATGGTGA TTCTGAAGTT GTTCTTAAAA
12201 AGTTGAAGAA GTCTTTGAAT GTGGCTAAAT CTGAATTTGA
12241 CCGTGATGCA GCCATGCAAC GTAAGTTGGA AAAGATGGCT
12281 GATCAAGCTA TGACCCAAAT GTATAAACAG GCTAGATCTG
12321 AGGACAAGAG GGCAAAAGTT ACTAGTGCTA TGCAGACAAT
12361 GCTTTTCACT ATGCTTAGAA AGTTGGATAA TGATGCACTC
12401 AACAACATTA TCAACAATGC AAGAGATGGT TGTGTTCCCT
12441 TGAACATAAT ACCTCTTACA ACAGCAGCCA AACTAATGGT
12481 TGTCATACCA GACTATAACA CATATAAAAA TACGTGTGAT
12521 GGTACAACAT TTACTTATGC ATCAGCATTG TGGGAAATCC
12561 AACAGGTTGT AGATGCAGAT AGTAAAATTG TTCAACTTAG
12601 TGAAATTAGT ATGGACAATT CACCTAATTT AGCATGGCCT
12641 CTTATTGTAA CAGCTTTAAG GGCCAATTCT GCTGTCAAAT
12681 TACAGAATAA TGAGCTTAGT CCTGTTGCAC TACGACAGAT
12721 GTCTTGTGCT GCCGGTACTA CACAAACTGC TTGCACTGAT
12761 GACAATGCGT TAGCTTACTA CAACACAACA AAGGGAGGTA
12801 GGTTTGTACT TGCACTGTTA TCCGATTTAC AGGATTTGAA
12841 ATGGGCTAGA TTCCCTAAGA GTGATGGAAC TGGTACTATC
12881 TATACAGAAC TGGAACCACC TTGTAGGTTT GTTACAGACA
12921 CACCTAAAGG TCCTAAAGTG AAGTATTTAT ACTTTATTAA
12961 AGGATTAAAC AACCTAAATA GAGGTATGGT ACTTGGTAGT
13001 TTAGCTGCCA CAGTACGTCT ACAAGCTGGT AATGCAACAG
13041 AAGTGCCTGC CAATTCAACT GTATTATCTT TCTGTGCTTT
13081 TGCTGTAGAT GCTGCTAAAG CTTACAAAGA TTATCTAGCT
13121 AGTGGGGGAC AACCAATCAC TAATTGTGTT AAGATGTTGT
13161 GTACACACAC TGGTACTGGT CAGGCAATAA CAGTTAGACC
13201 GGAAGCCAAT ATGGATCAAG AATCCTTTGG TGGTGCATCG
13241 TGTTGTCTGT ACTGCCGTTG CCACATAGAT CATCCAAATC
13281 CTAAAGGATT TTGTGACTTA AAAGGTAAGT ATGTACAAAT
13321 ACCTACAACT TGTGCTAATG ACCCTGTGGG TTTTACACTT
13361 AAAAACACAG TCTGTACCGT CTGCGGTATG TGGAAAGGTT
13401 ATGGCTGTAG TTGTGATCAA CTCCGCGAAC CCATGCTTCA
13441 GTCAGCTGAT GCACAATCGT TTTTAAACGG GTTTGCGGTG
13481 TAAGTGCAGC CCGTCTTACA CCGTGCGGCA CAGGCACTAG
13521 TACTGATGTC GTATACAGGG CTTTTGACAT CTACAATGAT
13561 AAAGTAGCTG GTTTTGCTAA ATTCCTAAAA ACTAATTGTT
13601 GTCGCTTCCA AGAAAAGGAC GAAGATGACA ATTTAATTGA
13641 TTCTTACTTT GTAGTTAAGA GACACACTTT CTCTAACTAC
13681 CAACATGAAG AAACAATTTA TAATTTACTT AAGGATTGTC
13721 CAGCTGTTGC TAAACATGAC TTCTTTAAGT TTAGAATAGA
13761 CGGTGACATG GTACCACATA TATCACGTCA ACGTCTTACT
13801 AAATACACAA TGGCAGACCT CGTCTATGCT TTAAGGCATT
13841 TTGATGAAGG TAATTGTGAC ACATTAAAAG AAATACTTGT
13881 CACATACAAT TGTTGTGATG ATGATTATTT CAATAAAAAG
13921 GACTGGTATG ATTTTGTAGA AAACCCAGAT ATATTACGCG
13961 TATACGCCAA CTTAGGTGAA CGTGTACGCC AAGCTTTGTT
14001 AAAAACAGTA CAATTCTGTG ATGCCATGCG AAATGCTGGT
14041 ATTGTTGGTG TACTGACATT AGATAATCAA GATCTCAATG
14081 GTAACTGGTA TGATTTCGGT GATTTCATAC AAACCACGCC
14121 AGGTAGTGGA GTTCCTGTTG TAGATTCTTA TTATTCATTG
14161 TTAATGCCTA TATTAACCTT GACCAGGGCT TTAACTGCAG
14201 AGTCACATGT TGACACTGAC TTAACAAAGC CTTACATTAA
14241 GTGGGATTTG TTAAAATATG ACTTCACGGA AGAGAGGTTA
14281 AAACTCTTTG ACCGTTATTT TAAATATTGG GATCAGACAT
14321 ACCACCCAAA TTGTGTTAAC TGTTTGGATG ACAGATGCAT
14361 TCTGCATTGT GCAAACTTTA ATGTTTTACT CTCTACAGTG
14401 TTCCCACCTA CAAGTTTTGG ACCACTAGTG AGAAAAATAT
14441 TTGTTGATGG TGTTCCATTT GTAGTTTCAA CTGGATACCA
14481 CTTCAGAGAG CTAGGTGTTG TACATAATCA GGATGTAAAC
14521 TTACATAGCT CTAGACTTAG TTTTAAGGAA TTACTTGTGT
14561 ATGCTGCTGA CCCTGCTATG CACGCTGCTT CTGGTAATCT
14601 ATTACTAGAT AAACGCACTA CGTGCTTTTC AGTAGCTGCA
14641 CTTACTAACA ATCTTGCTTT TCAAACTGTC AAACCCGGTA
14681 ATTTTAACAA AGACTTCTAT GACTTTGCTG TGTCTAAGGG
14721 TTTCTTTAAG GAAGGAAGTT CTGTTGAATT AAAACACTTC
14761 TTCTTTGCTC AGGATGGTAA TGCTGCTATC AGCGATTATG
14801 ACTAGTATCG TTATAATCTA CCAACAATGT GTGATATCAG
14841 ACAACTACTA TTTGTAGTTG AAGTTGTTGA TAAGTACTTT
14881 GATTGTTACG ATGGTGGCTG TATTAATGCT AACCAAGTCA
14921 TCGTCAACAA CCTAGACAAA TCAGCTGGTT TTCCATTTAA
14961 TAAATGGGGT AAGGCTAGAC TTTATTATGA TTCAATGAGT
15001 TATGAGGATC AAGATGCACT TTTCGCATAT ACAAAACGTA
15041 ATGTCATCCC TACTATAACT CAAATGAATC TTAAGTATGC
15081 CATTAGTGCA AAGAATAGAG CTCGCACCGT AGLTGGTGTC
15121 TCTATCTGTA GTACTATGAC CAATAGACAG TTTCATCAAA
15161 AATTATTGAA ATCAATAGCC GCCACTAGAG GAGCTACTGT
15201 AGTAATTGGA ACAAGCAAAT TCTATGGTGG TTGGCACAAC
15241 ATGTTAAAAA CTGTTTATAG TGATGTAGAA AACCCTCACC
15281 TTATGGGTTG GGATTATCCT AAATGTGATA GAGCCATGCC
15321 TAACATGCTT AGAATTATGG CCTCACTTGT TCTTGCTCGC
15361 AAACATACAA CGTGTTGTAG CTTGTCACAC CGTTTCTATA
15401 GATTAGCTAA TGAGTGTGCT CAAGTATTGA GTGAAATGGT
15441 CATGTGTGGC GGTTCACTAT ATGTTAAACC AGGTGGAACC
15481 TCATCAGGAG ATGCCACAAC TGCTTATGCT AATAGTGTTT
15521 TTAACATTTG TCAAGCTGTC ACGGCCAATG TTAATGCACT
15561 TTTATCTACT GATGGTAACA AAATTGCCGA TAAGTATGTC
15601 CGCAATTTAC AACACAGACT TTATGAGTGT CTCTATAGAA
15641 ATAGAGATGT TGACACAGAC TTTGTGAATG AGTTTTACGC
15681 ATATTTGCGT AAACATTTCT CAATGATGAT ACTCTCTGAC
15721 GATGCTGTTG TGTGTTTCAA TAGCACTTAT GCATCTCAAG
15761 GTCTAGTGGC TAGCATAAAG AACTTTAAGT CAGTTCTTTA
15801 TTATCAAAAC AATGTTTTTA TGTCTGAAGC AAAATGTTGG
15841 ACTGAGACTG ACCTTACTAA AGGACCTCAT GAATTTTGCT
15881 CTCAACATAC AATGCTAGTT AAACAGGGTG ATGATTATGT
15921 GTACCTTCCT TACCCAGATC CATCAAGAAT CCTAGGGGCC
15961 GGCTGTTTTG TAGATGATAT CGTAAAAACA GATGGTACAC
16001 TTATGATTGA ACGGTTCGTG TCTTTAGCTA TAGATGCTTA
16041 CCCACTTACT AAACATCCTA ATCAGGAGTA TGCTGATGTC
16081 TTTCATTTGT ACTTACAATA CATAAGAAAG CTACATGATG
16121 AGTTAACAGG ACACATGTTA GACATGTATT CTGTTATGCT
16161 TAGTAATGAT AACAGTTCAA GGTATTGGGA ACCTGAGTTT
16201 TATGAGGCTA TGTACACACC GCATACAGTC TTACAGGCTG
16241 TTGGGGCTTG TGTTCTTTGC AATTCACAGA CTTCATTAAG
16281 ATGTGGTGCT TGCATACGTA GACCATTCTT ATGTTGTAAA
16321 TGCTGTTACG ACCATGTCAT ATCAACATCA CATAAATTAG
16361 TCTTGTCTGT TAATCCGTAT GTTTGCAATG CTCCAGGTTG
16401 TGATGTCACA GATGTGACTC AACTTTACTT AGGAGGTATG
16441 AGCTATTATT GTAAATGAGA TAAACCACCC ATTAGTTTTC
16481 CATTGTGTGC TAATGGACAA GTTTTTGGTT TATATAAAAA
16521 TACATGTGTT GGTAGCGATA ATGTTACTGA CTTTAATGCA
16561 ATTGLAACAT GTGACTGGAC AAATGCTGGT GATTACATTT
16601 TAGCTAACAC CTGTACTGAA AGACTCAAGC TTTTTGCAGC
16641 AGAAACGCTC AAAGCTAGTG AGGAGACATT TAAACTGTCT
16681 TATGGTATTG CTACTGTACG TGAAGTGCTG TCTGACAGAG
16721 AATTACATCT TTCATGGGAA GTTGGTAAAC CTAGACCACC
16161 ACTTAACCGA AATTATGTCT TTACTGGTTA TCGTGTAACT
16801 AAAAACAGTA AAGTACAAAT AGGAGAGTAC ACCTTTGAAA
16841 AAGGTGAGTA TGGTGATGCT GTTGTTTACC GAGGTACAAC
16881 AACTTACAAA TTAAATGTTG GTGATTATTT TGTGCTGACA
16921 TCACATACAG TAATGCCATT AAGTGCACCT ACACTAGTGC
16961 CACAAGAGCA CTATGTTAGA ATTACTGGCT TATACCCAAC
17001 ACTCAATATC TCAGATGAGT TTTCTAGCAA TGTTGCAAAT
17041 TATCAAAAGG TTGGTATGCA AAAGTATTCT ACACTCCAGG
17081 GACCACCTGG TACTGGTAAG AGTCATTTTG CTATTGGCCT
17121 AGCTCTCTAC TACCCTTCTG CTCGCATAGT GTATACAGCT
17161 TGCTCTCATG CCGCTGTTGA TGCACTATGT GAGAAGGCAT
17201 TAAAATATTT GCCTATAGAT AAATGTAGTA GAATTATACC
17241 TGCACGTGCT CGTGTAGAGT GTTTTGATAA ATTCAAAGTG
17281 AATTCAACAT TAGAACAGTA TGTCTTTTGT ACTGTAAATG
17321 CATTGCCTGA GACGACAGCA GATATAGTTG TCTTTGATGA
17361 AATTTCAATG GCCACAAATT ATGATTTGAG TGTTGTCAAT
17401 GCCAGATTAC GTGCTAAGCA CTATGTGTAC ATTGGCGACC
17441 CTGCTCAATT ACCTGCACCA CGCACATTGC TAACTAAGGG
17481 CACACTAGAA CCAGAATATT TCAATTCAGT GTGTAGACTT
17521 ATGAAAACTA TAGGTCCAGA CATGTTCCTC GGAACTTGTC
17561 GGCGTTGTCC TGCTGAAATT GTTGACACTG TGAGTGCTTT
17601 GGTTTATGAT AATAAGCTTA AAGCACATAA AGACAAATCA
17641 GCTCAATGCT TTAAAATGTT TTATAAGGGT GTTATCACGC
17681 ATGATGTTTC ATCTGCAATT AACAGGCCAC AAATAGGCGT
17721 GGTAAGAGAA TTCCTTACAC GTAACCCTGC TTGGAGAAAA
17761 GCTGTCTTTA TTTCACCTTA TAATTCACAG AATGCTGTAG
17801 CCTCAAAGAT TTTGGGACTA CCAACTCAAA CTGTTGATTC
17841 ATCACAGGGC TCAGAATATG ACTATGTCAT ATTCACTCAA
17881 ACCACTGAAA CAGCTCACTC TTGTAATGTA AACAGATTTA
17921 ATGTTGCTAT TACCAGAGCA AAAGTAGGCA TACTTTGCAT
17961 AATGTCTGAT AGAGAGCTTT ATGACAAGTT GCAATTTACA
18001 AGTCTTGAAA TTCCACGTAG GAATGTGGCA ACTTTACAAG
18041 CTGAAAATGT AACAGGACTC TTTAAAGATT GTAGTAAGGT
18081 AATCACTGGG TTACATCCTA CACAGGCACC TACACACCTC
18121 AGTGTTGACA CTAAATTCAA AACTGAAGGT TTATGTGTTG
18161 ACATACCTGG CATACCTAAG GACATGACCT ATAGAAGACT
18201 CATCTCTATG ATGGGTTTTA AAATGAATTA TCAAGTTAAT
18241 GGTTACCCTA ACATGTTTAT CACCCGCGAA GAAGCTATAA
18281 GACATGTACG TGCATGGATT GGCTTCGATG TCGAGGGGTG
18321 TCATGCTACT AGAGAAGGTG TTGGTACCAA TTTACCTTTA
18361 CAGCTAGGTT TTTCTACAGG TGTTAACCTA GTTGCTGTAC
18401 CTACAGGTTA TGTTGATACA CCTAATAATA CAGATTTTTC
18441 CAGAGTTAGT GCTAAACCAC CGCCTGGAGA TCAATTTAAA
18481 CACCTCATAC CAGTTATGTA CAAAGGACTT CCTTGGAATG
18521 TAGTGCGTAT AAAGATTGTA CAAATGTTAA GTGACACACT
18561 TAAAAATCTC TGTGAGAGAG TCGTATTTGT CTTATGGGCA
18601 CATGGCTTTG AGTTGACATC TATGAAGTAT TTTGTGAAAA
18641 TAGGACCTGA GCGCACCTGT TGTCTATGTG ATAGACGTGC
18681 CACATGCTTT TCCACTGCTT CAGACACTTA TGCCTGTTGG
18721 CATCATTCTA TTGGATTTGA TTACGTCTAT AATCCGTTTA
18761 TGATTGATGT TCAACAATGG GGTTTTACAG GTAACCTACA
18801 AAGCAACCAT GATCTGTATT GTCAAGTCCA TGGTAATGCA
18841 CATGTAGCTA GTTGTGATGC AATCATGACT AGGTGTCTAG
18881 CTGTCCACGA GTGCTTTGTT AAGCGTGTTG ACTGGACTAT
18921 TGAATATCCT ATAATTGGTG ATGAACTGAA GATTAATGCG
18961 GCTTGTAGAA AGGTTCAACA CATGGTTGTT AAAGCTGCAT
19001 TATTAGCAGA CAAATTCCCA GTTCTTCACG ACATTGGTAA
19041 CCCTAAAGCT ATTAAGTGTG TACCTCAAGC TGATGTAGAA
19081 TGGAAGTTCT ATGATGCACA GCCTTGTAGT GACAAAGCTT
19121 ATAAAATAGA AGAATTATTC TATTCTTATG CCACACATTC
19161 TGACAAATTC AGAGATGGTG TATGCCTATT TTGGAATTGC
19201 AATGTCGATA GATATCCTGC TAATTCCATT GTTTGTAGAT
19241 TTGACACTAG AGTGCTATCT AACCTTAACT TGCCTGGTTG
19281 TGATGGTGGC AGTTTGTATG TAAATAAACA TGCATTCCAC
19321 ACACCAGCTT TTGATAAAAG TGCTTTTGTT AATTTAAAAC
19361 AATTACCATT TTTCTATTAC TCTGACAGTC CATGTGAGTC
19401 TCATGGAAAA CAAGTAGTGT GAGATATAGA TTATGTACCA
19441 CTAAAGTCTG CTACGTGTAT AACACGTTGC AATTTAGGTG
19481 GTGCTGTCTG TAGACATCAT GCTAATGAGT ACAGATTGTA
19521 TCTCGATGCT TATAACATGA TGATCTCAGC TGGCTTTAGC
19561 TTGTGGGTTT ACAAACAATT TGATACTTAT AACCTCTGGA
19601 ACACTTTTAG AAGACTTCAG AGTTTAGAAA ATGTGGCTTT
19641 TAATGTTGTA AATAAGGGAC ACTTTGATGG ACAACAGGGT
19681 GAAGTACCAG TTTCTATCAT TAATAACACT GTTTACACAA
19721 AAGTTGATGG TGTTGATGTA GAATTGTTTG AAAATAAAAC
19761 AACATTACCT GTTAATGTAG CATTTGAGCT TTGGGCTAAG
19801 CGCAACATTA AACCAGTACC AGAGGTGAAA ATACTCAATA
19841 ATTTGGGTGT GGACATTGCT GCTAATACTG TGATCTGGGA
19881 CTACAAAAGA GATGCTCCAG CACATATATC TACTATTGGT
19921 GTTTGTTCTA TGACTGACAT AGCCAAGAAA CCAACTGAAA
19961 CGATTTGTGC ACCACTCACT GTCTTTTTTG ATGGTAGAGT
20001 TGATGGTCAA GTAGACTTAT TTAGAAATGC CCGTAATGGT
20041 GTTCTTATTA CAGAAGGTAG TGTTAAAGGT TTACAACCAT
20081 CTGTAGGTCC CAAACAAGCT AGTCTTAATG GAGTGAGATT
20121 AATTGGAGAA GCCGTAAAAA CACAGTTCAA TTATTATAAG
20161 AAAGTTGATG GTGTTGTCCA ACAATTACCT GAAACTTACT
20201 TTACTCAGAG TAGAAATTTA CAAGAATTTA AACCCAGGAG
20241 TCAAATGGAA ATTGATTTCT TAGAATTAGC TATGGATGAA
20281 TTCATTGAAC GGTATAAATT AGAAGGCTAT GCCTTCGAAC
20321 ATATCGTTTA TGGAGATTTT AGTCATAGTC AGTTAGGTGG
20361 TTTACATCTA CTGATTGGAC TAGCTAAACG TTTTAAGGAA
20401 TCACCTTTTG AATTAGAAGA TTTTATTCCT ATGGACAGTA
20441 CAGTTAAAAA CTATTTCATA ACAGATGCGC AAACAGGTTC
20481 ATCTAAGTGT GTGTGTTCTG TTATTGATTT ATTAGTTGAT
20521 GATTTTGTTG AAATAATAAA ATCCCAAGAT TTATCTGTAG
20561 TTTCTAAGGT TGTCAAAGTG ACTATTGACT ATACAGAAAT
20601 TTCATTTATG CTTTGGTGTA AAGATGGCCA TGTAGAAACA
20641 TTTTACCCAA AATTACAATC TAGTCAAGCG TGGCAACCGG
20681 GTGTTGCTAT GCCTAATCTT TACAAAATGC AAAGAATGCT
20721 ATTAGAAAAG TGTGACCTTC AAAATTATGG TGATAGTGCA
20761 ACATTACCTA AAGGCATAAT GATGAATGTC GCAAAATATA
20801 CTCAACTGTG TCAATATTTA AACACATTAA CATTAGCTGT
20841 ACCCTATAAT ATGAGAGTTA TACATTTTGG TGCTGGTTCT
20881 GATAAAGGAG TTGCACCAGG TACAGCTGTT TTAAGACAGT
20921 GGTTGCCTAC GGGTACGCTG CTTGTCGATT CAGATCTTAA
20961 TGACTTTGTC TCTGATGCAG ATTCAACTTT GATTGGTGAT
21001 TGTGCAACTG TACATACAGC TAATAAATGG GATCTCATTA
21041 TTAGTGATAT GTACGACCCT AAGACTAAAA ATGTTACAAA
21081 AGAAAATGAC TCTAAAGAGG GTTTTTTCAC TTACATTTGT
21121 GGGTTTATAC AACAAAAGCT AGCTCTTGGA GGTTCCGTGG
21161 CTATAAAGAT AACAGAACAT TCTTGGAATG CTGATCTTTA
21201 TAAGCTCATG GGACACTTCG CATGGTGGAC AGCCTTTGTT
21241 ACTAATGTGA ATGCGTCATC ATCTGAAGCA TTTTTAATTG
21281 GATGTAATTA TCTTGGCAAA CCACGCGAAC AAATAGATGG
21321 TTATGTCATG CATGCAAATT ACATATTTTG GAGGAATACA
21361 AATCCAATTC AGTTGTCTTC CTATTCTTTA TTTGACATGA
21401 GTAAATTTCC CCTTAAATTA AGGGGTACTG CTGTTATGTC
21441 TTTAAAAGAA GGTCAAATCA ATGATATGAT TTTATCTCTT
21481 CTTAGTAAAG GTAGACTTAT AATTAGAGAA AACAACAGAG
21521 TTGTTATTTC TAGTGATGTT CTTGTTAACA ACTAAACGAA
21561 CAATGTTTGT TTTTCTTGTT TTATTGCCAC TAGTCTCTAG
21601 TCAGTGTGTT AATCTTACAA CCAGAACTCA ATTAGCCCCT
21641 GCATACACTA ATTCTTTCAC ACGTGGTGTT TATTACCCTG
21681 ACAAAGTTTT CAGATCCTCA GTTTTAGATT CAACTCAGGA
21721 CTTGTTCTTA CCTTTCTTTT CCAATGTTAC TTGGTTCCAT
21761 GCTATACATG TCTCTGGGAC CAATGGTACT AAGAGGTTTG
21801 ATAACCCTGT CCTACCATTT AATGATGGTG TTTATTTTGC
21841 TTCCACTGAG AAGTCTAACA TAATAAGAGG CTGGATTTTT
21881 GGTACTACTT TAGATTCGAA GACCCAGTCC CTACTTATTG
21921 TTAATAACGC TACTAATGTT GTTATTAAAG TCTGTGAATT
21961 TCAATTTTGT AATGATCCAT TTTTGGGTGT TTATTAGCAC
22001 AAAAACAACA AAAGTTGGAT GGAAAGTGAG TTCAGAGTTT
22041 ATTCTAGTGC GAATAATTGC ACTTTTGAAT ATGTCTCTCA
22081 GCCTTTTCTT ATGGACCTTG AAGGAAAACA GGGTAATTTC
22121 AAAAATCTTA GGGAATTTGT GTTTAAGAAT ATTGATGGTT
22161 ATTTTAAAAT ATATTCTAAG CACACGCCTA TTAATTTAGT
22201 GCGTGATCTC CCTCAGGGTT TTTCGGCTTT AGAACCATTG
22241 GTAGATTTGC CAATAGGTAT TAACATCACT AGGTTTCAAA
22281 CTTTACTTGC TTTACATAGA AGTTATTTGA CTCCTGGTGA
22321 TTCTTCTTCA GGTTGGACAG CTGGTGCTGC AGCTTATTAT
22361 GTGGGTTATC TTCAACCTAG GACTTTTCTA TTAAAATATA
22401 ATGAAAATGG AACCATTACA GATGCTGTAG ACTGTGCACT
22441 TGACCCTCTC TCAGAAACAA AGTGTACGTT GAAATCCTTC
22481 ACTGTAGAAA AAGGAATCTA TCAAACTTCT AACTTTAGAG
22521 TCCAACCAAC AGAATCTATT GTTAGATTTC CTAATATTAC
22561 AAACTTGTGC CCTTTTGGTG AAGTTTTTAA CGCCACCAGA
22601 TTTGCATCTG TTTATGCTTG GAACAGGAAG AGAATCAGCA
22641 ACTGTGTTGC TGATTATTCT GTCCTATATA ATTCCGCATC
22681 ATTTTCCACT TTTAAGTGTT ATGGAGTGTC TCCTACTAAA
22721 TTAAATGATC TCTGCTTTAC TAATGTCTAT GCAGATTCAT
22761 TTGTAATTAG AGGTGATGAA GTCAGACAAA TCGCTCCAGG
22801 GCAAACTGGA AAGATTGCTG ATTATAATTA TAAATTACCA
22841 GATGATTTTA CAGGCTGCGT TATAGCTTGG AATTCTAACA
22881 ATCTTGATTC TAAGGTTGGT GGTAATTATA ATTACCTGTA
22921 TAGATTGTTT AGGAAGTCTA ATCTCAAACC TTTTGAGAGA
22961 GATATTTCAA CTGAAATCTA TCAGGCCGGT AGCACACCTT
23001 GTAATGGTGT TGAAGGTTTT AATTGTTACT TTCCTTTACA
23041 ATCATATGGT TTCCAACCCA CTAATGGTGT TGGTTACCAA
23081 CCATACAGAG TAGTAGTACT TTCTTTTGAA CTTCTACATG
23121 CACCAGCAAC TGTTTGTGGA CCTAAAAAGT CTACTAATTT
23161 GGTTAAAAAC AAATGTGTCA ATTTCAACTT CAATGGTTTA
23201 ACAGGCACAG GTGTTCTTAC TGAGTCTAAC AAAAAGTTTC
23241 TGCCTTTCCA ACAATTTGGC AGAGACATTG CTGACACTAC
23281 TGATGCTGTC CGTGATCCAC AGACACTTGA GATTCTTGAC
23321 ATTAGACCAT GTTCTTTTGG TGGTGTCAGT GTTATAACAC
23361 CAGGAACAAA TACTTCTAAC CAGGTTGCTG TTCTTTATCA
23401 GGATGTTAAC TGCACAGAAG TCCCTGTTGC TATTCATGCA
23441 GATCAACTTA CTCCTACTTG GCGTGTTTAT TCTACAGGTT
23481 CTAATGTTTT TCAAACACGT GCAGGCTGTT TAATAGGGGC
23521 TGAACATGTC AACAACTCAT ATGAGTGTGA CATACCCATT
23561 GGTGCAGGTA TATGCGCTAG TTATCAGACT CAGACTAATT
23601 CTCCTCGGCG GGCACGTAGT GTAGCTAGTC AATCCATCAT
23641 TGCCTACACT ATGTCACTTG GTGCAGAAAA TTCAGTTGCT
23681 TACTCTAATA ACTCTATTGC CATACCCACA AATTTTACTA
23721 TTAGTGTTAC CACAGAAATT CTACCAGTGT CTATGACCAA
23761 GACATCAGTA GATTGTACAA TGTACATTTG TGGTGATTCA
23801 ACTGAATGCA GCAATCTTTT GTTGCAATAT GGCAGTTTTT
23841 GTACACAATT AAACCGTGCT TTAACTGGAA TAGCTGTTGA
23881 ACAAGACAAA AACACCLAAG AAGTTTTTGC ACAAGTCAAA
23921 CAAATTTACA AAACACCACC AATTAAAGAT TTTGGTGGTT
23961 TTAATTTTTC ACAAATATTA CCAGATCCAT CAAAACCAAG
24001 CAAGAGGTCA TTTATTGAAG ATCTACTTTT CAACAAAGTG
24041 ACACTTGCAG ATGCTGGCTT CATCAAACAA TATGGTGATT
24081 GCCTTGGTGA TATTGCTGCT AGAGACCTCA TTTGTGCACA
24121 AAAGTTTAAC GGCCTTACTG TTTTGCCACC TTTGCTCACA
24161 GATGAAATGA TTGCTCAATA CACTTCTGCA CTGTTAGCGG
24201 GTACAATCAC TTCTGGTTGG ACCTTTGGTG CAGGTGCTGC
24241 ATTACAAATA CCATTTGCTA TGCAAATGGC TTATAGGTTT
24281 AATGGTATTG GAGTTAGACA GAATGTTCTC TATGAGAACC
24321 AAAAATTGAT TGCCAACCAA TTTAATAGTG CTATTGGCAA
24361 AATTCAAGAC TCACTTTCTT CCACAGCAAG TGCACTTGGA
24401 AAACTTCAAG ATGTGGTCAA CCAAAATGCA CAAGCTTTAA
24441 ACACGCTTGT TAAACAACTT AGCTCCAATT TTGGTGCAAT
24481 TTCAAGTGTT TTAAATGATA TCCTTTCACG TCTTGACAAA
24521 GTTGAGGCTG AAGTGCAAAT TGATAGGTTG ATCACAGGCA
24561 GACTTCAAAG TTTGCAGACA TATGTGACTC AACAATTAAT
24601 TAGAGCTGCA GAAATCAGAG CTTCTGCTAA TCTTGCTGCT
24641 ACTAAAATGT CAGAGTGTGT ACTTGGACAA TCAAAAAGAG
24681 TTGATTTTTG TGGAAAGGGC TATCATCTTA TGTCCTTCCC
24721 TCAGTCAGCA CCTCATGGTG TAGTCTTCTT GCATGTGACT
24761 TATGTCCCTG CACAAGAAAA GAACTTCACA ACTGCTCCTG
24801 CCATTTGTCA TGATGGAAAA GCACACTTTC CTCGTGAAGG
24841 TGTCTTTGTT TCAAATGGCA CACACTGGTT TGTAACACAA
24881 AGGAATTTTT ATGAACCACA AATCATTACT ACAGACAACA
24921 CATTTGTGTC TGGTAACTGT GATGTTGTAA TAGGAATTGT
24961 CAACAACACA GTTTATGATC CTTTGCAACC TGAATTAGAC
25001 TCATTCAAGG AGGAGTTAGA TAAATATTTT AAGAATCATA
25041 CATCACCAGA TGTTGATTTA GGTGACATCT CTGGCATTAA
25081 TGCTTCAGTT GTAAACATTC AAAAAGAAAT TGACCGCCTC
25121 AATGAGGTTG CCAAGAATTT AAATGAATCT CTCATCGATC
25161 TCCAAGAACT TGGAAAGTAT GAGCAGTATA TAAAATGGCC
25201 ATGGTACATT TGGCTAGGTT TTATAGCTGG CTTGATTGCC
25241 ATAGTAATGG TGACAATTAT GCTTTGCTGT ATGACCAGTT
25281 GCTGTAGTTG TCTCAAGGGC TGTTGTTCTT GTGGATCCTG
25321 CTGCAAATTT GATGAAGACG ACTCTGAGCC AGTGCTCAAA
25361 GGAGTCAAAT TACATTACAC ATAAACGAAC TTATGGATTT
25401 GTTTATGAGA ATCTTCACAA TTGGAACTGT AACTTTGAAG
25441 CAAGGTGAAA TCAAGGATGC TACTCCTTCA GATTTTGTTC
25481 GCGCTACTGC AACGATAGCG ATACAAGCCT CACTCCCTTT
25521 CGGATGGCTT ATTGTTGGCG TTGCACTTCT TGCTGTTTTT
25561 CAGAGCGCTT CCAAAATCAT AACCCTCAAA AAGAGATGGC
25601 AACTAGCACT CTCCAAGGGT GTTCACTTTG TTTGCAACTT
25641 GCTGTTGTTG TTTGTAACAG TTTACTCAGA CCTTTTGCTC
25681 GTTGCTGCTG GCCTTGAAGC CCCTTTTCTC TATCTTTATG
25721 CTTTAGTCTA CTTCTTGCAG AGTATAAACT TTGTAAGAAT
25761 AATAATGAGG CTTTGGCTTT GCTGGAAATG CCGTTCCAAA
25801 AACCCATTAG TTTATGATGC CAACTATTTT CTTTGCTGGC
25841 ATACTAATTG TTACGACTAT TGTATACCTT ACAATAGTGT
25881 AACTTCTTCA ATTGTCATTA CTTCAGGTGA TGGCACAACA
25921 AGTCCTATTT CTGAACATGA CTACCAGATT GGTGGTTATA
25961 CTGAAAAATG GGAATCTGGA GTAAAAGACT GTGTTGTATT
26001 ACACAGTTAC TTCACTTCAG ACTATTACCA GCTGTACTCA
26041 ACTCAATTGA GTACAGACAC TGGTGTTGAA CATGTTACCT
26081 TCTTCATCTA CAATAAAATT GTTGATGAGC CTGAAGAACA
26121 TGTCCAAATT CACACAATCG ACGGTTCATC CGSAGTTGTT
26161 AATCCAGTAA TGGAACCAAT TTATGATGAA CCAACGACGA
26201 CTACTAGCGT GCCTTTGTAA GCACAAGCTG ATAAGTACGA
26241 ACTTATGTAG TCATTCGTTT CGGAAGAGAC AGGTACGTTA
26281 ATAGTTAATA GCGTACTTCT TTTTCTTGCT TTCGTGGTAT
26321 TCTTGCTAGT TACACTAGCC ATCCTTACTG CGCTTCGATT
26361 GTGTGCGTAC TGCTGCAATA TTGTTAACGT GAGTCTTGTA
26401 AAACCTTCTT TTTACGTTTA CTCTCGTGTT AAAAATCTGA
26441 ATTCTTCTAG AGTTCCTGAT CTTCTGGTCT AAACGAACTA
26481 AATATTATAT TAGTTTTTCT GTTTGGAACT TTAATTTTAG
26521 CCATGGCAGA TTCCAACGGT ACTATTACCG TTGAAGAGCT
26561 TAAAAAGCTC CTTGAACAAT GGAACCTAGT AATAGGTTTC
26601 CTATTCCTTA CATGGATTTG TCTTCTACAA TTTGCCTATG
26641 CCAACAGGAA TAGGTTTTTG TATATAATTA AGTTAATTTT
26681 CCTCTGGCTG TTATGGCCAG TAACTTTAGC TTCTTTTGTG
26721 CTTGCTGCTG TTTACAGAAT AAATTGGATC ACCGGTGGAA
26761 TTGCTATCGC AATGGCTTGT CTTGTAGGCT TGATGTGGCT
26801 CAGCTACTTC ATTGCTTCTT TCAGACTGTT TGCGCGTACG
26841 CGTTCCATGT GGTCATTCAA TCCAGAAACT AACATTCTTC
26881 TCAACGTGCC ACTCCATGGC ACTATTCTGA CCAGACCGCT
26921 TCTAGAAAGT GAACTCGTAA TCGGAGCTGT GATCCTTCGT
26961 GGACATCTTC GTATTGCTGG ACACCATCTA GGACGCTGTG
27001 ACATCAAGGA CCTGCCTAAA GAAATCACTG TTGCTACATC
27041 ACGAACGCTT TCTTATTACA AATTGGGAGC TTCGCAGCGT
27081 GTAGCAGGTG ACTCAGGTTT TGCTGCATAC AGTCGCTACA
27121 GGATTGGCAA CTATAAATTA AACACAGACC ATTCCAGTAG
27161 CAGTGACAAT ATTGCTTTGC TTGTACAGTA AGTGACAACA
27201 GATGTTTCAT CTCGTTGACT TTCAGGTTAC TATAGCAGAG
27241 ATATTACTAA TTATTATGAG GACTTTTAAA GTTTCCATTT
27281 GGAATCTTGA TTACATCATA AACCTCATAA TAAAAAATTT
27321 ATCTAAGTCA CTAACTGAGA ATAAATATTC TCAATTAGAT
27361 GAAGAGCAAC CAATGGAGAT TGATTAAACG AACATGAAAA
27401 TTATTCTTTT CTTGGCACTG ATAACACTCG CTACTTGTGA
27441 GCTTTATCAC TAGCAAGAGT GTGTTAGAGG TACAACAGTA
27481 CTTTTAAAAG AACCTTGCTC TTCTGGAACA TACGAGGGCA
27521 ATTCACCATT TCATCCTCTA GCTGATAACA AATTTGCACT
27561 GACTTGCTTT AGGACTCAAT TTGCTTTTGC TTGTCCTGAC
27601 GGCGTAAAAC ACGTCTATCA GTTACGTGCC AGATCAGTTT
27641 CACCTAAACT GTTCATGAGA CAAGAGGAAG TTCAAGAACT
27681 TTACTCTCCA ATTTTTCTTA TTGTTGCGGC AATAGTGTTT
27721 ATAACACTTT GCTTCACACT CAAAAGAAAG ACAGAATGAT
2/iol TGAACTTTCA TTAATTGACT TCTATTTGTG CTTTTTAGCC
27801 TTTCTGCTAT TCCTTGTTTT AATTATGCTT ATTATCTTTT
27841 GGTTCTCACT TGAACTGCAA GATCATAATG AAACTTGTCA
27881 CGCCTAAACG AACATGAAAT TTCTTGTTTT CTTAGGAATC
27921 ATCACAACTG TAGCTGCATT TCACCAAGAA TGTAGTTTAC
27961 AGTCATGTAG TCAACATCAA CCATATGTAG TTGATGACCC
28001 GTGTCCTATT CACTTCTATT CTAAATGGTA TATTAGAGTA
28041 GGAGCTAGAA AATCAGCACC TTTAATTGAA TTGTGCGTGG
28081 ATGAGGCTGG TTCTAAATCA CCCATTCAGT ACATCGATAT
28121 CGGTAATTAT ACAGTTTCCT GTTTACCTTT TAAAATTAAT
28161 TGCCAGGAAC CTAAATTGGG TAGTCTTGTA GTGCGTTGTT
28201 CGTTCTATGA AGACTTTTTA GAGTATCATG ACGTTCGTGT
28241 TGTTTTAGAT TTCATCTAAA CGAACAAACT AAAATGTCTG
28281 ATAATGGACC CCAAAATCAG CGAAATGCAC CCCGCATTAC
28321 GTTTGGTGGA CCCTCAGATT CAACTGGCAG TAACCAGAAT
28361 GGAGAACGCA GTGGGGCGCG ATCAAAACAA CGTCGGCCCC
28401 AAGGTTTACC CAATAATACT GCGTCTTGGT TCACCGCTCT
28441 CACTCAACAT GGCAAGGAAG ACCTTAAATT CCCTCGAGGA
28481 CAAGGCGTTC CAATTAACAC CAATAGCAGT CCAGATGACC
28521 AAATTGGCTA CTAGCGAAGA GCTACCAGAC GAATTCGTGG
28561 TGGTGACGGT AAAATGAAAG ATCTCAGTCC AAGATGGTAT
28601 TTCTACTACC TAGGAACTGG GCCAGAAGCT GGACTTCCCT
28641 ATGGTGCTAA CAAAGACGGC ATCATATGGG TTGCAACTGA
28681 GGGAGCCTTG AATACACCAA AAGATCACAT TGGCACCCGC
28721 AATCCTGCTA ACAATGCTGC AATCGTGCTA CAACTTCCTC
28761 AAGGAACAAC ATTGCCAAAA GGCTTCTACG CAGAAGGGAG
28801 CAGAGGCGGC AGTCAAGCCT CTTCTCGTTC CTCATCACGT
28841 AGTCGCAACA GTTCAAGAAA TTCAACTCCA GGCAGCAGTA
28881 GGGGAACTTC TCCTGCTAGA ATGGCTGGCA ATGGCGGTGA
28921 TGCTGCTCTT GCTTTGCTGC TGCTTGACAG ATTGAACCAG
28961 CTTGAGAGCA AAATGTCTGG TAAAGGCCAA CAACAACAAG
29001 GCCAAACTGT CACTAAGAAA TCTGCTGCTG AGGCTTCTAA
29041 GAAGCCTCGG CAAAAACGTA CTGCCACTAA AGCATACAAT
29081 GTAACACAAG CTTTCGGCAG ACGTGGTCCA GAACAAACCC
29121 AAGGAAATTT TGGGGACCAG GAACTAATCA GACAAGGAAC
29161 TGATTAGAAA CATTGGCCGC AAATTGCACA ATTTGCCCCC
29201 AGCGCTTCAG CGTTCTTCGG AATGTCGCGC ATTGGCATGG
29241 AAGTCACACC TTCGGGAACG TGGTTGACCT ACACAGGTGG
29281 CATCAAATTG GATGACAAAG ATCCAAATTT CAAAGATCAA
29321 GTCATTTTGC TGAATAAGCA TATTGACGCA TACAAAACAT
29361 TCCCACCAAC AGAGCCTAAA AAGGACAAAA AGAAGAAGGC
29401 TGATGAAACT CAAGCCTTAC CGCAGAGACA GAAGAAACAG
29441 CAAACTGTGA CTCTTCTTCC TGCTGCAGAT TTGGATGATT
29481 TCTCCAAACA ATTGCAACAA TCCATGAGCA GTGCTGACTC
29521 AACTCAGGCC TAAACTCATG CAGACCACAC AAGGCAGATG
29561 GGCTATATAA ACGTTTTCGC TTTTCCGTTT ACGATATATA
29601 GTCTACTCTT GTGCAGAATG AATTCTCGTA ACTACATAGC
29641 ACAAGTAGAT GTAGTTAACT TTAATCTCAC ATAGCAATCT
29681 TTAATCAGTG TGTAACATTA GGGAGGACTT GAAAGAGCCA
29721 CCACATTTTC ACCGAGGCCA CGCGGAGTAC GATCGAGTGT
29761 ACAGTGAACA ATGCTAGGGA GAGCTGCCTA TATGGAAGAG
29801 CCCTAATGTG TAAAATTAAT TTTAGTAGTG CTATCCCCAT
29841 GTGATTTTAA TAGCTTCTTA GGAGAATGAC AAAAAAAAAA
29881 AAAAAAAAAA AAAAAAAAAA AAA
```

The SARS-CoV-2 can have a 5′ untranslated region (5′ UTR; also known as a leader sequence or leader RNA) at positions 1-265 of the SEQ CD NO:1 sequence. Such a 5′ UTR can include the region of an mRNA that is directly upstream from the initiation codon. The 5′ UTR and 3′ UTR may also facilitate packaging of SARS-CoV-2.

Similarly, the SARS-CoV-2 can have a 3′ untranslated region (3′ UTR) at positions 29675-29903. In positive strand RNA viruses, the 3′-UTR can play a role in viral RNA replication because the origin of the minus-strand RNA replication intermediate is at the 3′-end of the genome.

The SARS-CoV-2 genome encodes four major structural proteins: the spike (S) protein, nucleocapsid (N) protein, membrane (M) protein, and the envelope (E) protein. Some of these proteins are part of a large polyprotein, which is at positions 266-21555 of the SEQ ID NO:1 sequence, where this open reading frame is referred to as ORFlab polyprotein and has SEQ ID NO:2, shown below.

```
      1 MESLVPGENE KTHVQLSLPV LQVRDVLVRG FGDSVEEVLS
    41 EARQHLKDGT CGLVEVEKGV LPQLEQPYVF IKRSDARTAP
    81 HGHVMVELVA ELEGIQYGRS GETLGVLVPH VGEIPVAYRK
  121 VLLRKNGNKG AGGHSYGADL KSFDLGDELG TDPYEDFQEN
  161 WNTKHSSGVT RELMRELNGG AYTRYVDNNF CGPDGYPLEC
  201 IKDLLARAGK ASCTLSEOLD FIDTKRGVYC CREHEHEIAW
  241 YTERSEKSYE LQTPFEIKLA KKFDTFNGEC PNFVFPLNST
  281 IKTIQPRVEK KKLDGFMGRI RSVYPVASPN ECNQMCLSTL
  321 MKCDHCGETS WQTGDFVKAT CEFCGTENLT KEGATTCGYL
  361 PQNAVVKIYC PACHNSEVGP EHSLAEYHNE SGLKTILRKG
  401 GRTIAFGGCV FSYVGCHNKC AYWVPRASAN IGCNHTGVVG
  441 EGSEGLNDNL LEILQKEKVN INIVGDFKLN EEIAIILASF
  481 SASTSAFVET VKGLDYKAFK QIVESCGNFK VTKGKAKKGA
  521 WNIGEQKSIL SPLYAFASEA ARVVRSIFSR TLETAQNSVR
  561 VLQKAAITIL DGISQYSLRL IDAMMFTSDL ATNNLVVMAY
  601 ITGGVVQLTS QWLTNIFGTV YEKLKPVLDW LEEKFKEGVE
  641 FLRDGWEIVK FISTCACEIV GGQIVTCAKE IKESVQTFFK
  681 LVNKFLALCA DSIIIGGAKL KALNLGETFV THSKGLYRKC
  721 VKSREETGLL MPLKAPKEII FLEGETLPTE VLTEEVVLKT
  761 GDLQPLEQPT SEAVEAPLVG TPVCINGLML LEIKDTEKYC
  801 ALAPNMMVTN NTFTLKGGAP TKVTFGDDTV IEVQGYKSVN
  841 ITFELDERID KVLNEKCSAY TVELGTEVNE FACVVADAVI
  881 KTLQPVSELL TPLGIDLDEW SMATYYLFDE SGEFKLASHM
  921 YCSFYPPDED EEEGDCEEEE FEPSTQYEYG TEDDYQGKPL
  961 EFGATSAALQ PEEEQEEDWL DDDSQQTVGQ QDGSEDNQTT
1001 TIQTIVEVQP QLEMELTPVV QTTEVNSFSG YLKLTDNVYI
1041 KNADIVEEAK KVKPTVVVNA ANVYLKHGGG VAGALNKATN
1081 NAMQVESDDY IATNGPLKVG GSCVLSGHNL AKHCLHVVGP
1121 NVNKGEDIQL LKSAYENFNQ HEVLLAPLLS AGIFGADPIH
1161 SLRVCVDTVR TNVYLAVFDK NLYDKLVSSF LEMKSEKQVE
1201 QKIAEIPKEE VKPFITESKP SVEQRKQDDK KIKACVEEVT
1241 TTLEETKFLT ENLLLYIDIN GNLHPDSATL VSDIDITFLK
1281 KDAPYIVGDV VQEGVLTAVV IPTKKAGGTT EMLAKALRKV
1321 PTDNYITTYP GQGLNGYTVE EAKTVLKKCK SAFYILPSII
1361 SNEKQEILGT VSWNLREMLA HAEETRKLMP VCVETKAIVS
1401 TIQRKYKGIK IQEGVVDYGA RFYFYTSKTT VASLINTLND
1441 LNETLVTMPL GYVTHGLNLE EAARYMRSLK VPATVSVSSP
1481 DAVTAYNGYL TSSSKTPEEH FIETISLAGS YKDWSYSGQS
1521 TQLGIEFLKR GDKSVYYTSN PTTFHLDGEV ITFDNLKTLL
1581 SLREVRTIKV FTTVDNINLH TQVVDMSMTY GQQFGPTYLD
1601 GADVTKIKPH NSHEGKTFYV LPNDDTLRVE AFEYYHTTDP
1641 SFLGRYMSAL NHTKKWKYPQ VNGLTSIKWA DNNCYLATAL
1681 LTLQQIELKF NPPALQDAYY RARAGEAANF CALILAYCNK
1721 TVGELGDVRE TMSYLFQHAN LDSCKRVLNV VCKTCGQQQT
1761 TLKGVEAVMY MGTLSYEQFK KGVQIPCTCG KQATKYLVQQ
1801 ESPFVMMSAP PAQYELKHGT FTCASEYTGN YQCGHYKHIT
1841 SKETLYCIDG ALLTKSSEYK GPITDVFYKE NSYTTTIKPV
1881 TYKLDGVVCT EIDPKLDNYY KKDNSYFTEQ PIDLVPNQPY
1921 PNASFDNFKF VCDNIKFADD LNOLTGYKKP ASRELKVTFF
1961 PDLNGDVVAI DYKHYTPSFK KGAKLLHKPI VWHVNNATNK
2001 ATYKPNTWCI RCLWSTKPVE TSNSFDVLKS EDAQGMDNLA
2041 CEDLKPVSEE VVENPTIQKD VLECNVKTTE VVGDIILKPA
2081 NNSLKITEEV GHTDLMAAYV DNSSLTIKKP NELSRVLGLK
2121 TLATHGLAAV NSVPWDTIAN YAKPFLNKVV STTTNTVTRC
2161 LNRVCTNYMP IFFTLLLQLC TFTRSTNSRI KASMPTTIAK
2201 NTVKSVGKFC LEASFNYLKS PNFSKLINII IWFLLLSVCL
2241 GSLIYSTAAL GVLMSNLGMP SYCTGYREGY LNSTNVTIAT
2281 YCTGSIPCSV CLSGLDSLDT YPSLETIQIT ISSFKWDLTA
2321 FGLVAEWFLA YILFTRFFYV LGLAAIMQLF FSYFAVHFIS
2361 NSWLMWLIIN LVOMAPISAM VRMYIFFASF YYVWKSYVHV
2401 VDGCNSSTCM MCYKRNRATR VECTTIVNGV RRSFYVYANG
2441 GKGFCKLHNW NCVNCDTFCA GSTFISDEVA RDLSLQFKRP
2481 INPTDQSSYI VDSVTVKNGS IHLYFDKAGQ KTYERHSLSH
2521 FVNLDNLRAN NTKGSLPINV IVFDGKSKCE ESSAKSASVY
2561 YSOLMCOPIL LLDQALVSDV GDSAEVAVKM FDAYVNTFSS
2601 TFNVPMEKLK TLVATAEAEL AKNVSLDNVL STFISAARQG
2641 FVDSDVETKD VVECLKLSHQ SDIEVTGDSC NNYMLTYNKV
2481 ENMTPRDLGA CIDCSAREIN AQVAKSHNIA LIWNVKDFMS
2521 LSEQLRKQIR SAAKKNNLPF KLTCATTRQV VNVVTTKIAL
2561 KGGKIVNNWL KQLIKVTLVF LFVAAIFYLI TPVHVMSKHT
2601 DFSSEIIGYK AIDGGVTRDI ASTDTCFANK HADFDTWFSQ
2641 RGGSYTNDKA CPLIAAVITR EVGFVVPGLP GTILRTTNGD
2681 FLHFLPRVFS AVGNICYTPS KLIEYTDFAT SACVLAAECT
2721 IFKDASGKPV PYCYDTNVLE GSVAYESLRP DTRYVLMDGS
2761 IIQFPNTYLE GSVRVVTTFD SEYCRHGTCE RSEAGVCVST
2801 SGRWVLNNDY YRSLPGVFCG VDAVNLLTNM FTPLIQPIGA
2841 LDISASIVAG GIVAIVVTCL AYYFMRFRRA FGEYSHVVAF
2881 NTLLFLMSFT VLCLTPVYSF LPGVYSVIYL YLTFYLTNDV
2921 SFLAHIQWMV MFTPLVPFW1 TIAYIICIST KHFYWFFSNY
2961 LKRRVVFNGV SFSTFEEAAL CTFLLNKEMY LKLRSDVLLP
3001 LTQYNRYLAL YNKYKYFSGA MDTTSYREAA CCHLAKALND
3041 FSNSGSDVLY QPPQTSITSA VLQSGFRKMA FPSGKVEGCM
3081 VQVTCGTTTL NGLWLDDVVY CPRHVICTSE DMLNPNYEDL
3121 LIRKSNHNFL VQAGNVQLRV IGHSMQNCVL KLKVDTANPK
3161 TPKYKFVRIQ PGQTFSVLAC YNGSPSGVYQ CAMRPNFTIK
3201 GSFLNGSCGS VGFNIDYDCV SFCYMHHMEL PTGVHAGTDL
3241 EGNFYGPFVD RQTAQAAGTD TTiTVNVLAW LYAAVINGDK
3281 WFLNRFTTTL NDFNLVAMKY NYEPLTQDHV DILGPLSAQT
3321 GIAVLDMCAS LKELLQNGMN GRTILGSALL EDEFTPFDVV
3361 RQCSGVTFQS AVKRTIKGTH HWLLLTILTS LLVLVQSTQW
3401 SLFFFLYENA FLPFAMGIIA MSAFAMMFVK HKHAFLCLFL
3441 LPSLATVAYE NMVYMPASWV MRIMTWLDMV DTSLSGFKLK
3481 DCVMYASAVV LLILMTARTV YDDGARRVWT LMNVLTLVYK
3521 VYYGNALDQA ISMWALIISV TSNYSGVVTT VMFLARGIVE
3561 MCVEYCPIFF ITGNTLQCIM LVYCFLGYFC TCYFGLFCLL
3601 NRYFRLTLGV YDYLVSTQEF RYMNSQGLLP PKNSIDAFKL
3641 NIKLLGVGGK PCIKVATVOS KMSDVKCTSV VLLSVLQQLR
3681 VESSSKLWAQ CVQLHNDILL AKDTTEAFEK MVSLLSVLLS
3721 MQGAVDINKL CEEMLDNRAT LQAIASEFSS LPSYAAFATA
3761 QEAYEQAVAN GDSEVVLKKL KKSLNVAKSE FDRDAAMQRK
3801 LEKMADQAMT QMYKQARSED KRAKVTSAMQ TMLFTMLRKL
3841 DNDALNNIIN NARDGCVPLN IIPLTTAAKL MVVTPDYNTY
3881 KNTCDGTTFT YASALWEIQQ VVDADSKIVQ LSEISMDNSP
3921 NLAWPLIVTA LRANSAVKLQ NNELSPVALR QMSCAAGTTQ
3961 TACTDDNALA YYNTTKGGRF VLALLSDLOD LKWARFPKSD
4001 GTGTIYTELE PPCRFVTDTP KGPKVKYLYF IKGLNNLNRG
4041 MVLGSLAATV RLQAGNATEV PANSTVLSEC AFAVDAAKAY
4081 KDYLASGGQP ITNCVKMLCT HTGTGQAITV TPEANMDQES
4121 FGGASCCLYC RCHTDHPNPK GFCDLKGKYV QTPTTCANDP
4161 VGFTLKNTVC TVCGMWKGYG CSCDQLREPM LQSADAQSFL
4201 NG FAV
```

In some cases, the constructs and therapeutic interfering particles described herein can have a deletion of the SARS-CoV-2 genome that includes portions of the genome that encode SEQ ID NO:2. Such deletions can inactivate the SEQ ID NO:2 protein.

An RNA-dependent RNA polymerase is encoded at positions 13442-13468 and 13468-16236 of the SARS-CoV-2 SEQ ID NO:1 nucleic acid. This RNA-dependent RNA polymerase has been assigned NCBI accession number YP_009725307 and has the following sequence (SEQ ID NO:3).

```
    1 SADAQSFLNR VCGVSAARLT PCGTGTSTDV VYRAFDIYND
  41 KVAGFAKFLK TNCCRFQEKD EDDNLIDSYF VVKRHTFSNY
  81 QHEETIYNLL KDCPAVAKHD FFKFRIDGDM VPHISRQRLT
121 KYTMADLVYA LRHFDEGNCD TLKEILVTYN CCDDDYFNKK
161 DWYDFVENPD ILRVYANLGE RVRQALLKTV QFCDAMRNAG
201 IVGVLTLDNQ DLNGNWYDFG DFTQTTPGSG VPVVDSYYSL
241 LMPILTLTRA LTAESHVDTD LIKPYIKWDL LKYDFTEERL
281 KLFDRYFKYW DQTYHPNCVN CLDDRCILHC ANFNVLFSTV
321 FPPTSFGPLV RKIFVDGVPF VVSTGYHFRE LGVVHNQDVN
361 LHSSRLSFKE LLVYAADPAM HAASGNLLLD KRTTCFSVAA
401 LTNNVAFQTV KPGNFNKDFY DFAVSKGFFK EGSSVELKHF
441 FFAQDGNAAI SDYDYYRYNL PTMCDIRQLL FVVEVVDKYF
481 DCYDGGC1NA NQVIVNNLDK SAGFPFNKWG KARLYYDSMS
521 YEDODALFAY TKRNVIPTIT QMNLKYAISA KNRARTVAGV
561 SICSTMTNRQ FHQKLLKSIA ATRGATVVIG TSKFYGGWHN
601 MLKTVYSDVE NPHLMGWDYP KCDRAMPNML RIMASLVLAR
641 KHTTCCSLSH RFYRLANECA QVLSEMVMCG GSLYVKPGGT
681 SSGDATTAYA NSVFNICQAV TANVNALLST DGNKIADKYV
721 RNLQHRLYEC LYRNRDVDTD FVNEFYAYLR KHFSMMILSD
761 DAVVCFNSTY ASQGLVASIK NFKSVLYYON NVFMSEAKCW
801 TETDLTKGPH EFCSQHTMLV KQGDDYVYLP YPDPSRILGA
841 GCFVDDIVKT DGTLMIERFV SLAIDAYPLT KHPNQEYADV
881 FHLYLQYIRK LHDELTGHML DMYSVMLTND NTSRYWEPEF
921 YEAMYTPHTV LQ
```

In some cases, the constructs and therapeutic interfering particles described herein can have a deletion of the SARS-CoV-2 genome that includes portions of the genome that encode SEQ ID NO:3. Such deletions can inactivate the SEQ ID NO:3 protein.

A helicase is encoded at positions 16237-18039 of the SARS-CoV-2 SEQ ID NO:1 nucleic acid. This helicase has been assigned NCBI accession number YP_009725308.1 and has the following sequence (SEQ ID NO:4).

```
    1 AVGACVLCNS QTSLRCGACI RRPFLCCKCC YDHVISTSHK
  41 LVLSVNPYVC NAPGCDVTDV TQLYLGGMSY YCKSHKPPIS
  81 FPLCANGQVF GLYKNTCVGS DNVTDFNAIA TCDWTNAGDY
121 ILANTCTERL KLFAAETLKA TEETFKLSYG IATVREVLSD
161 RELHLSWEVG KPRPPLNRNY VFTGYRVTKN SKVQIGEYTE
201 EKGDYGDAVV YRGTTTYKLN VGDYFVLTSH TVMPLSAPTL
241 VPQEHYVRIT GLYPTLNISD EFSSNVANYQ KVGMQKYSTL
281 QGPPGTGKSH FAIGLALYYP SARIVYTAGS HAAVDALCEK
321 ALKYLPIDKC SRIIPARARV ECFDKFKVNS TLEQYVFCTV
361 NALPETTADI VVFDEISMAT NYDLSVVNAR LRAKHYVYIG
401 DPAQLPAPRT LLTKGTLEPE YFNSVCRLMK TIGPDMFLGT
441 CRRCPAEIVD TVSALVYDNK LKAHKDKSAQ CFKMFYKGVI
481 THDVSSAINR PQIGVVREFL TRNPAWRKAV FISPYNSQNA
521 VASKILGLPT QTVDSSQGSE YDYVIFTQTT ETAHSCNVNR
561 FNVAITRAKV GILCIMSDRD LYDKLQFTSL EIPRRNVATL
601 Q
```

In some cases, the constructs and therapeutic interfering particles described herein can have a deletion of the SARS-CoV-2 genome that includes portions of the genome that encode SEQ ID NOA4 Such deletions can inactivate the SEQ ID NOA4 protein.

The SARS-CoV-2 can have an open reading frame at positions 21563-25384 (gene 5) of the SEQ ID NO:1 sequence that can be referred to as GU280_gp02, where this open reading frame encodes a surface glycoprotein or a spike glycoprotein (SEQ ID NO:5, shown below).

| | |
|---|---|
| 1 | MEVFLVLLPL VSSQCVNLTT RTQLPPAYTN SFTRGVYYPD |
| 41 | KVFRSSVLHS TQDLFLPFFS NVTWFHAIHV SGTNGTKRFD |
| 81 | NPVLPFNDGV YFASTEKSNI IRGWIFGTTL DSKTQSLLIV |
| 121 | NNATNVVIKV CEFQFCNDPF LGVYYHKNNK SWMESEFRVY |
| 161 | SSANNCTFEY VSQPFLMDLE GKQGNFKNLR EFVFKNIDGY |
| 201 | FKIYSKHTPI NLVRDLPQGE SALEPLVDLP IGINITRFQT |
| 241 | LLALHRSYLT PGDSSSGWTA GAAAYYVGYL QPRTFLLKYN |
| 281 | ENGTITDAVD CALDPLSETK CTLKSFTVEK GIYQTSNFRV |
| 321 | QPTESIVRFP NITNLCPFGE VFNATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDFT GCVIAWNSNN |
| 441 | LDSKVGGNYN YLYRLFRKSN LKPFERDIST E1YQAGSTPC |
| 481 | NGVEGFNCYF PLQSYGFQPT NGVGYQPYRV VVLSFELLHA |
| 521 | PATVCGPKKS TNLVKNKCVN FNFNGLTGTG VLTESNKKIL |
| 561 | PFQQFGRDIA DTTDAVRDPQ TLEILDITPC SFGGVSVITP |
| 601 | GTNTSNQVAV LYQDVNCTEV PVAIHADQLT PTWRVYSTGS |
| 641 | NVFQTRAGCL IGAEHVNNSY ECDIPIGAGI CASYQTQTNS |
| 681 | PRRARSVASQ SIIAYTMSLG AENSVAYSNN SIAIPTNFTI |
| 721 | SVTTEILPVS MTKTSVDCTM YICGDSTECS NLLLQYGSFC |
| 761 | TQLNRALTGI AVEQDKNTQE VFAQVKQIYK TPPIKDFGGF |
| 801 | NFSQILPDPS KPSKRSFIED LLFNKVTLAD AGFTKQYGDC |
| 841 | LGDIAARDLI CAQKFNGLTV LPPLLTDEMI AQYTSALLAG |
| 881 | TITSGWTFGA GAALQIPFAM QMAYRFNGIG VTQNVLYENQ |
| 921 | KLIANQFNSA IGKIQDSLSS TASALGKLQD VVNQNAQALN |
| 961 | TLVKQLSSNF GAISSVLND1 LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNFTTAPA |
| 1081 | ICHDGKAHFP REGVFVSNGT HWFVTQKNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL HYT |

In some cases, the constructs and therapeutic interfering particles described herein can have a deletion of the SARS-CoV-2 genome that includes portions of the genome that encode SEQ ID NO:5. Such deletions can inactivate the SEQ ID NO:5 protein.

The S or spike protein is responsible for facilitating entry of the SARS-CoV-2 into cells. It is composed of a short intracellular tail, a transmembrane anchor, and a large ectodomain that consists of a receptor binding S1 subunit and a membrane-fusing S2 subunit. The spike receptor binding domain can reside at amino acid positions 330-583 of the SEQ ID NO:5 spike protein (shown below as SEQ ID NO:6).

330 P NITNLCPFGE VFNATRFASV YAWNRKRISN
361 CVADYSVLYN SASFSTEKCY GVSPTKLNDL CFTNVYADSE
401 VIRGDEVRQI APGQTGKIAD YNYKLPDDFT GCVIAWNSNN
441 LDSKVGGNYN YLYRLFRKSN LKPFERDIST EIYQAGSTPC
481 NGVEGFNCYF PLQSYGFQPT NGVGYQPYRV VVLSFELLHA
521 PATVCGPKKS TNLVKNKCVN FNFNGLTGTG VLTESNKKFL
561 PFQQFGRDIA DTTDAVRDPQ TLE

Analysis of this receptor binding motif (RBM) in the spike protein showed that most of the amino acid residues essential for receptor binding were conserved between SARS-CoV and SARS-CoV-2, suggesting that the 2 CoV strains use the same host receptor for cell entry. The entry receptor utilized by SARS-CoV is the angiotensin-converting enzyme 2 (ACE-2).

In some cases, the constructs and therapeutic interfering particles described herein can have a deletion of the SARS-CoV-2 genome that includes portions of the genome that encode SEQ ID NO:6. Such deletions can inactivate the SEQ ID NO:6 protein.

The SARS-CoV-2 spike protein membrane-fusing S2 domain can be at positions 662-1270 of the SEQ ID NO:5 spike protein (shown below as SEQ ID NO:7).

```
  662 CDIPIGAGI CASYQTQTNS
  681 PRRARSVASQ SIIAYTMSLG AENSVAYSNN SIAIPTNFTI
  721 SVTTEILPVS MTKTSVDCTM YICGDSTECS NLLLQYGSFC
  761 TQLNRALTGI AVEQDKNTQE VFAQVKQIYK TPPIKDFGGF
  801 NFSQILPDPS KPSKRSFTED LLFNKVTLAD AGFIKQYGDC
  841 LGDIAARDLI CAQKFNGLTV LPPLLTDEMI AQYTSALLAG
  881 TITSGWTFGA GAALQIPFAM QMAYRFNGIG VTQNVLYENQ
  921 KLIANQFNSA IGKIQDSLSS TASALGKLQD VVNQNAQALN
  961 TLVKQLSSNF GAISSVLNDI LSRLDKVEAE VQIDRLITGR
1001 LQSLQTYVTQ QLIRAAEIRA SANLAATKMS ECVLGQSKRV
1041 DFCGKGYHLM SFPQSAPHGV VFLHVTYVPA QEKNFTTAPA
1081 ICHDGKAHFP REGVFVSNGT HWFVTQRNFY EPQIITTDNT
1121 FVSGNCDVVI GIVNNTVYDP LQPELDSFKE ELDKYFKNHT
1161 SPDVDLGDIS GINASVVNIQ KEIDRLNEVA KNLNESLIDL
1201 QELGKYEQYI KWPWYIWLGF IAGLIAIVMV TIMLCCMTSC
1241 CSCLKGCCSC GSCCKFDEDD SEPVLKGVKL H
```

The SARS-CoV-2 can have an open reading frame at positions 2720-8554 of the SEQ ID NO:1 sequence that can be referred to as nsp3, which includes transmembrane domain 1 (TM1). This nsp3 open reading frame with transmembrane domain 1 has NCBI accession no. YP_009725299.1 and is shown below as SEQ ID NO:8.

| | |
|---|---|
| 1 | APTKVTFGDD TVIEVQGYKS VNITFELDER IDKVLNEKCS |
| 41 | AYTVELGTEV NEFACVVADA VIKTLQPVSE LLTPLGIDLD |
| 81 | EWSMATYYLF DESGEFKLAS HMYCSFYPPD EDEEEGDCEE |
| 121 | EEFEPSTQYE YGTEDDYQGK PLEFGATSAA LQPEEEQEED |
| 161 | WLDDDSQQTV GQQDGSEDNQ TTTIQTIVEV QPQLEMELTP |
| 201 | VVQTIEVNSF SGYLKLTDNV YIKNADIVEE AKKVKPTVVV |
| 241 | NAANVYLKHG GGVAGALNKA TNNAMQVESD DYIATNGPLK |
| 281 | VGGSCVLSGH NLAKHCLHVV GPNVNKGEDI QLLKSAYENF |
| 321 | NQHEVLLAPL LSAGIFGADP IHSLRVCVDT VRTNVYLAVF |
| 361 | DKNLYDKLVS SFLEMKSEKQ VEQKIAEIPK EEVKPFITES |
| 401 | KPSVEQRKQD DKKIKACVEE VTTTLEETKF LTENLLLYID |
| 441 | INGNLHPDSA TLVSDIDITF LKKDAPYIVG DVVQEGVLTA |
| 481 | VVIPTKKAGG TTEMLAKALR KVPTDNYITT YPGQGLNGYT |
| 521 | VEEAKTVLKK CKSAFYILPS IISNEKQEIL GTVSWNLREM |
| 561 | LAHAEETRKL MPVCVETKAI VSTIQRKYKG IKIQEGVVDY |
| 601 | GARFYFYTSK TTVASLINTL NDLNETLVTM PLGYVTHGLN |
| 641 | LEEAARYMRS LKVPATVSVS SPDAVTAYNG YLTSSSKTPE |
| 681 | EHFIETISLA GSYKDWSYSG QSTQLGIEFL KRGDKSVYYT |
| 721 | SNPTTFHLDG EVITFDNLKT LLSLREVRTI KVFTTVDNIN |
| 761 | LHTQVVDMSM TYGQQFGPTY LDGADVTKIK PHNSHEGKTF |
| 801 | YVLPNDDTLR VEAFEYYHTT DPSFLGRYMS ALNHTKKWKY |
| 841 | PQVNGLTSIK WADNNCYLAT ALLTLQQIEL KFNPPALQDA |
| 881 | YYRARAGEAA NFCALILAYC NKTVGELGDV RETMSYLFQH |
| 921 | ANLDSCKRVL NVVCKTCGQQ QTTLKGVEAV MYMGTLSYEQ |
| 961 | FKKGVQIPCT CGKQATKYLV QQESPFVMMS APPAQYELKH |
| 1001 | GTFTCASEYT GNYQCGHYKH ITSKETLYCI DGALLTKSSE |
| 1041 | YKGPITDVFY KENSYTTTIK PVTYKLDGVV CTEIDPKLDN |
| 1081 | YYKKDNSYFT EQPIDLVPNQ PYPNASFDNF KFVCDNIKFA |
| 1121 | DDLNQLTGYK KPASRELKVT FFPDLNGDVV AIDYKHYTPS |
| 1161 | FKKGAKLLHK PIVWHVNNAT NKATYKPNTW CIRCLWSTKP |
| 1201 | VETSNSFDVL KSEDAQGMDN LACEDLKPVS EEVVENPTIQ |
| 1241 | KDVLECNVKT TEVVGDITLK PANNSLKITE EVGHTDLMAA |
| 1281 | YVDNSSLTIK KPNELSRVLG LKTLATHGLA AVNSVPWDTI |
| 1321 | ANYAKPFLNK VVSTTTNIVT RCLNRVCTNY MPYFFTLLLQ |
| 1361 | LCTFTRSTNS RIKASMPTTI AKNTVKSVGK FCLEASFNYL |
| 1401 | KSPNFSKLIN IIIWFLLLSV CLGSLIYSTA ALGVLMSNLG |
| 1441 | MPSYCTGYRE GYLNSTNVTI ATYCTGSIPC SVCLSGLDSL |
| 1481 | DTYPSLETIQ ITISSFKWDL TAFGLVAEWF LAYILFTRFF |
| 1521 | YVLGLAAIMQ LFFSYFAVHF ISNSWLMWLI INLVQMAPIS |
| 1561 | AMVRMYIFFA SFYYVWKSYV HVVDGCNSST CMMCYKRNRA |
| 1601 | TRVECTTIVN GVRRSFYVYA NGGKGFCKLH NWNCVNCDTF |
| 1641 | CAGSTFISDE VARDLSLQFK RPINPTDQSS YIVDSVTVKN |
| 1681 | GSIHLYFDKA GQKTYERHSL SHFVNLDNLR ANNTKGSLPI |
| 1721 | NVIVFDGKSK CEESSAKSAS VYYSQLMCQP ILLLDQALVS |
| 1761 | DVGDSAEVAV KMFDAYVNTF SSTFNVPMEK LKTLVATAEA |
| 1801 | ELAKNVSLDN VLSTFISAAR QGFVDSDVET KDVVECLKLS |
| 1841 | HQSDIEVTGD SCNNYMLTYN KVENMTPRDL GACIDCSARH |
| 1881 | INAQVAKSHN IALIWNVKDF MSLSEQLRKQ IRSAAKKNNL |
| 1921 | PFKLTCATTR QVVNVVTTKI ALKGG |

The nsp3 protein has additional conserved domains including an N-terminal acidic (Ac), a predicted phosphoesterase, a papain-like proteinase, Y-domain, transmembrane domain 1 (TM1), and an adenosine diphosphate-ribose 1″-phosphatase (ADRP).

In some cases, the constructs and therapeutic interfering particles described herein can have a deletion of the SARS-CoV-2 genome that includes portions of the genome that encode SEQ ID NO:8. Such deletions can inactivate the SEQ ID NO:8 protein.

The SARS-CoV-2 can have an open reading frame at positions 8555-10054 of the SEQ ID NO:1 sequence that can be referred to as nsp4B_TM, which includes transmembrane domain 2 (TM2). This nsp4B_TM open reading frame with transmembrane domain 2 has NCBI accession no. YP_009725300 and is shown below as SEQ ID NO:9.

```
    1 KIVNNWLKQL IKVTLVFLEV AAIFYLITPV HVMSKHTDFS
  41 SEIIGYKAID GGVTRDIAST DTCFANKHAD FDTWFSQRGG
  81 SYTNDKACPL IAAVITREVG FVVPGLPGTI LRTTNGDFLH
121 FLPRVFSAVG NICYTPSKLI EYTDFATSAC VLAAECTIFK
161 DASGKPVPYC YDTNVLEGSV AYESLRPDTR YVLMDGSIIQ
201 FPNTYLEGSV RVVTTFDSEY CRHGTCERSE AGVCVSTSGR
241 WVLNNDYYRS LPGVFCGVDA VNLLTNMFTP LIQPIGALDI
281 SASIVAGGIV AIVVTCLAYY FMRFRRAFGE YSHVVAFNTL
321 LFLMSFTVLC LTPVYSFLPG VYSVIYLYLT FYTTNDVSFL
361 AHIQWMVMFT PLVPFWITIA YIICISTKHF YWFFSNYLKR
401 RVVFNGVSFS TFEEAALCTF LLNKEMYLKL RSDVLLPLTQ
441 YNRYLALYNK YKYFSGAMDT TSYREAACCH LAKALNDFSN
481 SGSDVLYQPP QTSITSAVLQ
```

In some cases, the constructs and therapeutic interfering particles described herein can have a deletion of the SARS-CoV-2 genome that includes portions of the genome that encode SEQ ID NO:9. Such deletions can inactivate the SEQ ID NO:9 protein.

The SARS-CoV-2 can have an open reading frame at positions 25393-26220 (ORF3a) of the SEQ ID NO:1 sequence that can be referred to as GU280_gp03 (SEQ ID NO:10, shown below).

```
    1 MDLEMRIFTI GTVTLKQGEI KDATPSDFVR ATATIPIQAS
  41 LPFGWLIVGV ALLAVFQSAS KIITLKKRWQ LALSKGVHFV
  81 CNLLLLFVTV YSHLLLVAAG LEAPFLYLYA LVYFLQSINF
121 VRIIMRLWLC WKCRSKNPLL YDANYFLCWH TNCYDYCIPY
161 NSVTSSIVIT SGDGTTSPIS EHDYQIGGYT EKWESGVKDC
201 VVTHSYFTST YYQLYSTQLS TDTGVEHVTF FIYNKIVDEP
241 EEHVQIHTID GSSGVVNPVM EPIYDEPTTT TSVPL
```

In some cases, the constructs and therapeutic interfering particles described herein can have a deletion of the SARS-CoV-2 genome that includes portions of the genome that encode SEQ ID NO:10. Such deletions can inactivate the SEQ ID NO:10 protein.

The SARS-CoV-2 can have an open reading frame at positions 26245-26472 (gene E) of the SEQ ID NO:1 sequence that can be referred to as GU280_gp04 (SEQ ID NO: 11, shown below).

1 MYSFVSEETG TLIVNSVLLF LAFVVFLLVT LAILTALRLC
41 AYCCNIVNVS LVKPSFYVYS RVKNLNSSRV PDLLV

The SEQ ID NO: 11 protein is a structural protein, for example, an envelope protein. In some cases, the constructs and therapeutic interfering particles described herein can have a deletion of the SARS-CoV-2 genome that includes portions of the genome that encode SEQ ID NO:11. Such deletions can inactivate the SEQ ID NO:11 protein.

The SARS-CoV-2 can have an open reading frame at positions 27202-27191 (M protein gene; ORF5) of the SEQ ID NO:1 sequence that can be referred to as GU280_gp05 (SEQ ID NO:12, shown below).

```
    1 MADSNGTITV EELKKLLEQ WNLVIGFLFLT WICLLQFAYA
  41 NRNRFLYIIK LIFLWLLWP VTLACFVLAAV YRINWITGGI
121 A1AMACLVGL MWLSYFIAS FRLFARTRSMW SFNPETNILL
161 NVPLHGTILT RPLLESELV IGAVILRGHLR IAGHHLGRCD
201 IKDLPKEITV ATSRTLSYY KLGASQRVAGD SGFAAYSRYR
241 IGNYKLNTDH SSSSDNIA
121 LLVQ
```

The SEQ ID NO:12 protein is a structural protein, for example, a membrane glycoprotein. In some cases, the constructs and therapeutic interfering particles described herein can have a deletion of the SARS-CoV-2 genome that includes portions of the genome that encode SEQ ID NO:12. Such deletions can inactivate the SEQ ID NO:12 protein.

The SARS-CoV-2 can have an open reading frame at positions 27202-27387 (ORF6) of the SEQ ID NO:1 sequence that can be referred to as GU280_gp06 (SEQ ID NO:13, shown below).

1 MFHLVDFQVT IAEILLIIMR TFKVSIWNLD YIINLIIKNL
41 SKSLTENKYS QLDEEQPMEI D

In some cases, the constructs and therapeutic interfering particles described herein can have a deletion of the SARS-CoV-2 genome that includes portions of the genome that encode SEQ ID NO:13. Such deletions can inactivate the SEQ ID NO:13 protein.

The SARS-CoV-2 can have an open reading frame at positions 27394-27759 (ORF7a) of the SEQ ID NO:1 sequence that can be referred to as GU280_gp07 (SEQ ID NO:14, shown below).

```
    1 MKIILFLALI TLATCELYHY QECVRGTTVL LKEPCSSGTY
  41 EGNSPFHPLA DNKFALTCFS TQFAFACPDG VKHVYQLRAR
121 SVSPKLFIRQ EEVQELYSPI FLIVAAIVFI TLCFTLKRKT
161 E
```

In some cases, the constructs and therapeutic interfering particles described herein can have a deletion of the SARS-CoV-2 genome that includes portions of the genome that encode SEQ ID NO:14. Such deletions can inactivate the SEQ ID NO:14 protein.

The SARS-CoV-2 can have an open reading frame at positions 27756-27887 (ORF7b) of the SEQ ID NO:1 sequence that can be referred to as GU280_gp08 (SEQ ID NO:15, shown below).

1 MIELSLIDFY LCFLAFLLFL VLIMLIIFWF SLELQDHNET
41 CHA

In some cases, the constructs and therapeutic interfering particles described herein can have a deletion of the SARS-CoV-2 genome that includes portions of the genome that encode SEQ ID NO:15. Such deletions can inactivate the SEQ ID NO:15 protein.

The SARS-CoV-2 can have an open reading frame at positions 27894-28259 (ORF8) of the SEQ ID NO:1 sequence that can be referred to as GU280_gp09 (SEQ ID NO:16, shown below).

```
    1 MKFTVFLGII TTVAAFHQEC SLQSCTQHQP YVVDDPCPIH
  41 FYSKWYIRVG ARKSAPLIEL CVDEAGSKSP IQYIDIGNYT
121 VSCLPFTINC QEPKLGSLVV RCSFYEDFLE YHDVRVVLDF
161
```

In some cases, the constructs and therapeutic interfering particles described herein can have a deletion of the SARS-CoV-2 genome that includes portions of the genome that encode SEQ ID NO:16. Such deletions can inactivate the SEQ ID NO:16 protein.

The SARS-CoV-2 can have an open reading frame at positions 28274-29533 (gene N; ORF9) of the SEQ ID NO:1 sequence that can be referred to as GU280_gp10 (SEQ ID NO:17, shown below).

```
    1 MSDNGPQNQR NAPRITFGGP SDSTGSNQNG ERSGARSKQR
  41 RPQGLPNNTA SWFTALTQHG KEDLKFPRGQ GVPINTNSSP
121 DDQIGYYRRA TRRIRGGDGK MKDLSPRWYF YYLGTGPEAG
161 LPYGANKDGI 1WVATEGALN TPKDHIGTRN PANNAAIVLQ
201 LPQGTTLPKG FYAEGSRGGS QASSRSSSRS RNSSRNSTPG
241 SSRGTSPARM AGNGGDAALA LLLLDRLNQL ESKMSGKGQQ
281 QQGQTVTKKS AAEASKKPRQ KRTATKAYNV TQAFGRRGPE
521 QTQGNFGDQE LIRQGTDYKH WPQIAQFAPS ASAFFGMSRI
561 GMEVTPSGTW LTYTGAIKLD DKDPNFKDQV ILLNKHIDAY
601 KTFPPTEPKK DKKKKADETQ ALPQRQKKQQ TVTLLPAADL
641 DDFSKQLQQS MSSADSTQA
```

The SEQ ID NO 17 protein is a structural protein, for example, a nucleocapsid phosphoprotein. In some cases, the constructs and therapeutic interfering particles described herein can have a deletion of the SARS-CoV-2 genome that includes portions of the genome that encode SEQ ID NO:17. Such deletions can inactivate the SEQ ID NO:17 protein.

The SARS-CoV-2 can have an open reading frame at positions 29558-29674 (ORF10) of the SEQ ID NO:1 sequence that can be referred to as GU280_gp11 (SEQ ID NO:19, shown below).

1 MGYINVFAFP FTIYSLLLCR MNSRNYIAQV DWNFNLT

In some cases, the constructs and therapeutic interfering particles described herein can have a deletion of the SARS-CoV-2 genome that includes portions of the genome that encode SEQ ID NO:19. Such deletions can inactivate the SEQ ID NO:19 protein.

The SARS-CoV-2 can have a stem-loops at positions 29609-29644 and 29629-29657, which is within the encoded GU280_gp11. For example, the SARS-CoV-2 stem-loop at positions 29609-29644 is shown below as SEQ ID NO:20.

29601 TT GTGCAGAATG AATTCTCGTA ACTACATAGC
29641 ACAA

For example, the SARS-CoV-2 stem-loop at positions 29629-29657 is shown below as SEQ ID NO:21.

29629 TA ACTACATAGC ACAAGTAGAT GTAGTTA

In some cases, the constructs and therapeutic interfering particles described herein can have a deletion of the SARS-CoV-2 genome that includes portions of the genome that encode SEQ ID NO:20 and/or 21. Such deletions can inactivate the SEQ ID NO:20 and/or 21 protein.

The SARS-CoV-2 can have an open reading frame at positions 12686-13024 (nsp9) of the SEQ ID NO:1 sequence that encodes a ssRNA-binding protein with NCBI accession number YP_009725305.1, which has the following sequence (SEQ ID NO:22).

1 NNELSPVALR QMSCAAGTTQ TACTDDNALA YYNTTKGGRF
41 VLALLSDLQD LKWARFPKSD GTGTIYTELE PPCRFVTDTP
81 KGPKVKYLYF IKGLNNLNRG MVLGSLAATV RLQ

In some cases, the constructs and therapeutic interfering particles described herein can have a deletion of the SARS-CoV-2 genome that includes portions of the genome that encode SEQ ID NO:22. Such deletions can inactivate the SEQ ID NO:22 protein.

The constructs and/or therapeutic interfering particles described herein can have portions of the SARS-CoV-2 genome, where the deletions of the genome include at least 100, at least 500, at least 1000, at least 1500, at least 2000, at least 2500, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, at least 20,000, at least 21,000, at least 22,000, at least 23,000, at least 24,000, at least 25,000, at least 26,000, at least 27,000, at least 27500, or at least 28000 nucleotides of the SARS-CoV-2 genome.

The foregoing sequences are DNA sequences. The SARS-CoV-2 nucleic acids used in the compositions and methods described herein can be DNA or RNA versions of such sequences. The 3′ SARS-CoV-2 nucleic acids can include extended poly A sequences. For example, the extended poly-A sequences can have at least 100 adenine nucleotides to 250 adenine nucleotides. Such extended poly-A sequences can, for example, extend the half-life of the mRNA.

In addition, the SARS-CoV-2 genome can naturally have structural variations that are reflections of sequence variations. Hence, the SARS-CoV-2 used in the compositions and methods described herein can, for example, have one or more nucleotide or amino acid differences from the sequences shown as SEQ ID NO:1-35. In some cases, the SARS-CoV-2 used in the compositions and methods described herein can, for example, have two, three, four, five, six, seven, eight, nine, ten, fifteen, twenty, twenty-five, thirty, or more nucleotide or amino acid differences from the sequences shown as SEQ ID NO:1-35. Hence, prior to deletion any of the SARS-CoV-2 nucleic acids used in the methods and compositions described herein can be a DNA or RNA with at least 70%, or at least 75%, or at least 80%, or at least 85%, or at least 90%, or at least 95%, or at least 96%, or at least 97%, or at least 98%, or at least 99%, or at least 99.5% sequence identity to any of SEQ ID NO:1-35.

### SARS-CoV-2 Deletion Mutants

The present disclosure provides SARS-CoV-2 deletion mutants, for example, interfering, conditionally replicating, SARS-CoV-2 deletion mutants, and related constructs. For example, the present disclosure provides SARS-CoV-2 deletion mutants have one or more of the deletions relative to the wild type SARS-CoV-2 sequence.

The present disclosure therefore also provides SARS-CoV-2 deletion mutants. Such SARS-CoV-2 deletion mutants can have one or more deletions, for example at any location in SEQ ID NO:1. Such deletions can truncate or eliminate the sequence of any of the encoded polypeptides. For example, such deletions can truncate or delete the amino acid sequences identified by SEQ ID NOs: 2-19 or 22. For example, such deletions of SARS-CoV-2 nucleic acids can reduce or eliminate the expression of any of the polypeptides encoded by the SARS-CoV-2 nucleic acids. However, in some cases certain regions of the SARS-CoV-2 genome should be retained (e.g., portions of the 5′UTR and/or the 3′UTR) and not be deleted.

The present disclosure identifies specific regions of the SARS-CoV-2 genome that should be retained and specific regions of the SARS-CoV-2 genome that can be deleted in order to provide interfering, conditionally replicating, SARS-CoV-2 deletion mutants and related constructs. For example, in order to function as therapeutic interfering particles (TIPs), SARS-CoV-2 deletion mutants can retain cis-acting elements such as, for example, the 5′ UTR and the 3′ UTR. In addition to retaining cis-acting elements, the interfering SARS-CoV-2 particles can, in some cases, retain portions of some of the SARS-CoV-2 proteins, such as the N protein or the spike receptor binding S1 subunit (e.g., SEQ ID NO:6).

Interfering SARS-CoV-2 particles that exhibit interference with wild type SARS-CoV-2 may, for example, compete for structural proteins that mediate viral particle assembly, or produce proteins that inhibit assembly of viral particles. For example, interfering SARS-CoV-2 particles that exhibit interference can have a deletion in the membrane-fusing S2 subunit of the spike protein (e.g., SEQ ID NO:7). In some cases, interfering SARS-CoV-2 particles that exhibit interference can have one or more deletions in the RNA-dependent RNA polymerase (e.g., SEQ ID NO:3). In some cases, interfering SARS-CoV-2 particles that exhibit interference can have one or more deletions in the M protein (membrane glycoprotein)(e.g., SEQ ID NO:12). In some cases, interfering SARS-CoV-2 particles that exhibit interference can have one or more deletions in the ssRNA-binding protein (e.g., SEQ ID NO:22).

Also described herein are methods of generating a variant interfering, conditionally replicating, SARS-CoV-2 construct. The method generally involves: a) introducing an interfering construct as described above into a first host cell population or a first individual; b) obtaining a biological sample from a second cell population or a second individual to whom the interfering construct has been transmitted from the first host cell population or first individual (either directly or via one or more intervening cells/individuals), wherein the construct present in the second cell population or second individual is a variant of the interfering construct introduced into the first host cell population or first individual, and c) cloning the variant construct from the second host cell population or second individual.

The deletion sizes of the SARS-CoV-2 deletion mutants and interfering, conditionally replicating, SARS-CoV-2 construct can vary. For example, the SARS-CoV-2 deletion mutants and interfering, conditionally replicating, SARS-CoV-2 construct can have one or more deletions, where each deletion has at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 15 bp, at least 20 bp, at least 25 bp, at least 30 bp, at least 40 bp of deletion.

In some cases, the deletion size can range, for example, from about 10 bp to about 5000 bp; from about 800 bp to about 2500 bp; from about 900 bp to about 2400 bp; from about 1000 bp to about 2300 bp; from about 1100 bp to about 2200 bp; from about 1200 bp to about 2100 bp; from about 1300 bp to about 2000 bp; from about 1400 bp to about 1900 bp; from about 1500 bp to about 1800 bp; or from about 1600 bp to about 1700 bp.

The present disclosure provides an interfering, conditionally replicating SARS-CoV-2 construct. For simplicity, the interfering, conditionally replicating SARS-CoV-2 constructs are referred to as SARS-CoV-2 “interfering constructs” or “TIPs.” A subject interfering construct can be conditionally replicating. For example, a subject interfering construct, when present in a mammalian host, cannot, in the absence of a wild-type SARS-CoV-2, form infectious particles containing copies of itself. A subject interfering construct can be packaged into an infectious particle in vitro in a laboratory (e.g., in an in vitro cell culture) when the appropriate polypeptides required for packaging are provided. The infectious particle can deliver the interfering construct into a host cell, for example, an in vivo host cell. Once inside an in vivo host cell (a host cell in a mammalian subject), the interfering construct can integrate into the genome of the host cell or the interfering construct can remain cytoplasmic. The interfering construct can in some cases replicate in the in vivo host cell only in the presence of a wildtype SARS-CoV-2. When an in vivo host cell with an interfering construct is infected by a wildtype SARS-CoV-2, the interfering construct can replicate (e.g., is transcribed and packaged). In some cases, the interfering construct can replicate substantially more efficiently than the wildtype SARS-CoV-2, thereby outcompeting the wildtype SARS-CoV-2. As a result, the SARS-CoV-2 viral load is substantially reduced in the individual.

An interfering construct can be an RNA construct, or a DNA construct (e.g., a DNA copy of an RNA).

In some cases, an interfering construct does not include any heterologous nucleotide sequences not derived from SARS-CoV-2. “Heterologous” refers to a nucleotide sequence that is not normally present in a wild-type SARS-CoV-2 in nature. For example, in some cases an interfering construct may not include any heterologous nucleotide sequences that encode a gene product. Gene products include polypeptides and RNA.

In some cases an interfering construct can include heterologous nucleotide sequences not derived from SARS-CoV-2. For example, an interfering construct can include one or more barcode sequences, one or more segments encoding a detectable marker, one or more promoters, one or more RNA transcription or translation initiation sites, one or more termination signals, or a combination thereof. The constructs can also include an origin of replication.

An interfering construct can include SARS-CoV-2 cis-acting elements; and can include an alteration in the SARS-CoV-2 nucleotide sequence such that alteration renders one or more encoded SARS-CoV-2 trans-acting polypeptides non-functional. By “non-functional” is meant that the SARS-CoV-2 trans-activating polypeptide does not carry out its normal function, for example, due to truncation of or internal deletion within the encoded polypeptide, or due to lack of the polypeptide altogether. “Alteration” of a SARS-CoV-2 nucleotide sequence includes deletion of one or more nucleotides and/or substitution of one or more nucleotides.

In some cases, an interfering construct, when present in a host cell (e.g., in a host cell in an individual) that is infected with a wildtype SARS-CoV-2, replicates at a rate that is at least about 10%, at least about 20%, at least about 30%, at least about 40/6, at least about 50%, at least about 75%, at least about 2-fold, at least about 2.5-fold, at least about 5-fold, at least about 10-fold, or greater than 10-fold, higher than the rate of replication of the wildtype SARS-CoV-2 in a host cell of the same type that does not comprise a subject interfering construct.

In some cases, an interfering construct, when present in a host cell (e.g., in a host cell in an individual) that is infected with a wildtype SARS-CoV-2, reduces the amount of wildtype SARS-CoV-2 transcripts in the cell by at least about 20%, at least about 30%, at least about 40%, at least about 50%, at least about 60%, at least about 70%, at least about 80%, or at least about 90%, compared to the amount of wildtype SARS-CoV-2 transcripts in a host cell that is infected with wildtype SARS-CoV-2, but does not comprise a subject interfering construct.

In some cases, an interfering construct, when present in a host cell (e.g., in a host cell in an individual) that is infected with a wildtype SARS-CoV-2, results in production of interfering construct-encoded RNA such that the ratio (by weight, e.g., μg:μg) of interfering construct-encoded RNA to wild-type SARS-CoV-2-encoded RNA in the cytoplasm of the host cell is greater than 1. In some cases, an interfering construct, when present in a host cell (e.g., in a host cell in an individual) that is infected with a wildtype SARS-CoV-2, results in production of interfering construct-encoded RNA such that the ratio (by weight, e.g., μg:μg) of interfering construct-encoded RNA to wild-type SARS-CoV-2-encoded RNA in the cytoplasm of the host cell is from at least about 1.5:1 to at least about 102:1 or greater than 102:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

In some cases, an interfering construct, when present in a host cell (e.g., in a host cell in an individual) that is infected with a wildtype SARS-CoV-2, results in production of interfering construct-encoded RNA such that the ratio (e.g., molar ratio) of interfering construct-encoded RNA to wild-type SARS-CoV-2-encoded RNA in the cytoplasm of the host cell is greater than 1. In some cases, an interfering construct, when present in a host cell (e.g., in a host cell in an individual) that is infected with a wildtype SARS-CoV-2, results in production of interfering construct-encoded RNA such that the ratio (e.g., molar ratio) of interfering construct-encoded RNA to wild-type SARS-CoV-2-encoded RNA in the cytoplasm of the host cell is from at least about 1.5:1 to at least about 102:1 or greater than 102:1, e.g., from about 1.5:1 to about 2:1, from about 2:1 to about 5:1, from about 5:1 to about 10:1, from about 10:1 to about 25:1, from about 25:1 to about 50:1, from about 50:1 to about 75:1, from about 75:1 to about 100:1, or greater than 100:1.

A subject interfering construct can exhibit a basic reproductive ratio (R₀) (also referred to as the “basic reproductive number”) that is greater than 1. R₀ is the number of cases one case generates on average over the course of its infectious period. When R₀ is >1, the infection will be able to spread in a population (of cells or individuals). Thus, a subject interfering construct has the capacity to spread from one cell to another or from one individual to another in a population. In some cases, the subject interfering construct (or a subject interfering particle) has an R₀ from about 2 to about 5, from about 5 to about 7, from about 7 to about 10, from about 10 to about 15, or greater than 15.

Any convenient method can be used to measure the ratio of interfering construct-encoded RNA to wild-type SARS-CoV-2-encoded RNA in the cytoplasm of the host cell. Suitable methods can include, for example, measuring transcript number directly via qRT-PCR (e.g., single-cell qRT-PCR) of both an interfering construct-encoded RNA and a wild-type SARS-CoV-2-encoded RNA; measuring levels of a protein encoded by the interfering construct-encoded RNA and the wild-type SARS-CoV-2-encoded RNA (e.g., via western blot, ELISA, mass spectrometry, etc.); and measuring levels of a detectable label associated with the interfering construct-encoded RNA and the wild-type SARS-CoV-2-encoded RNA (e.g., fluorescence of a fluorescent protein that is encoded by the RNA and is fused to a protein that is translated from the RNA). Such measurements can be performed, for example, after co-transfection, using any convenient cell type.

In some embodiments, the interfering construct-encoded RNA is packaged. In some embodiments, the interfering construct-encoded RNA is unpackaged. In some cases, the interfering construct-encoded RNA includes both packaged and unpackaged RNA.

### Treatment

The present disclosure provides a method of reducing SARS-CoV-2 viral load in an individual. The method generally involves administering to the individual an effective amount of a subject interfering nucleic acid construct, a pharmaceutical formulation comprising a subject interfering nucleic acid construct, a subject interfering particle, or a pharmaceutical formulation comprising a subject interfering particle.

In some cases, a subject method involves administering to an individual in need thereof an effective amount of a SARS-CoV-2 interfering particle, or a pharmaceutical formulation comprising a subject interfering particle. In some cases, an effective amount of a subject interfering particle is an amount that, when administered to an individual in one or more doses, in monotherapy or in combination therapy, is effective to reduce SARS-CoV-2 virus load in the individual by at least about 10%, at least about 20%, at least about 25%, at least about 30%, at least about 40%, at least about 50%, at least about 60%, at least about 70%, at least about 80%, or greater than 80%, compared to the SARS-CoV-2 virus load in the individual in the absence of treatment with the interfering particle.

In some cases, a subject method involves administering to an individual in need thereof an effective amount of a subject interfering particle. In some embodiments, an “effective amount” of a subject interfering particle is an amount that, when administered to an individual in one or more doses, in monotherapy or in combination therapy, is effective to reduce symptoms of SARS-CoV-2 in the individual by at least about 20%, at least about 25%, at least about 30%, at least about 40%, at least about 50%, at least about 60%, at least about 70%, at least about 80%, at least about 2-fold, at least about 2.5-fold, at least about 3-fold, at least about 5-fold, at least about 10-fold, or greater than 10-fold, compared to the individual in the absence of treatment with the interfering particle.

Any of a variety of methods can be used to determine whether a treatment method is effective. For example, determining whether the methods are effective can include evaluating whether the wild type SARS-CoV-2 viral load is reduced, determining whether the infected subject is producing antibodies against SARS-CoV-2, determining whether the infected subject is breathing without assistance, and/or determining whether the temperature of the infected subject is returning to normal. Measuring viral load can be by measuring the amount of SARS-CoV-2 in a biological sample, for example, using a polymerase chain reaction (PCR) with primers specific SARS-CoV-2 polynucleotide sequence; detecting and/or measuring a polypeptide encoded by SARS-CoV-2; using an immunological assay such as an enzyme-linked immunosorbent assay (ELISA) with an antibody specific for a SARS-CoV-2 polypeptide; or a combination thereof.

### Formulations, Dosages, and Routes of Administration

Prior to introduction into a host, an interfering construct or an interfering particle can be formulated into various compositions for use in therapeutic and prophylactic treatment methods. In particular, the interfering construct or interfering particle can be made into a pharmaceutical composition by combination with appropriate pharmaceutically acceptable carriers or diluents and can be formulated to be appropriate for either human or veterinary applications. For simplicity, a subject interfering construct and a subject interfering particle are collectively referred to below as “active agent” or “active ingredient.”

Thus, a composition for use in a subject treatment method can comprise a SARS-CoV-2 interfering construct or SARS-CoV-2 interfering particle in combination with a pharmaceutically acceptable carrier. A variety of pharmaceutically acceptable carriers can be used that are suitable for administration. The choice of carrier will be determined, in part, by the particular vector, as well as by the particular method used to administer the composition. One skilled in the art will also appreciate that various routes of administering a composition are available, and, although more than one route can be used for administration, a particular route can provide a more immediate and more effective reaction than another route. Accordingly, there are a wide variety of suitable formulations of a subject interfering construct composition or a subject interfering particle composition.

A composition a subject interfering construct or subject interfering particle, alone or in combination with other antiviral compounds, can be made into a formulation suitable for parenteral administration. Such a formulation can include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be provided in unit dose or multidose sealed containers, such as ampules and vials, and can be stored in a freeze-dried (lyophilized) condition requiring only the addition of the sterile liquid carrier, for example, water, for injections, immediately prior to use. Injectable solutions and suspensions can be prepared from sterile powders, granules, and tablets, as described herein.

An aerosol formulation suitable for administration via inhalation also can be made. The aerosol formulation can be placed into a pressurized acceptable propellant, such as dichlorodifluoromethane, propane, nitrogen, and the like.

A formulation suitable for oral administration can be a liquid solution, such as an effective amount of a subject interfering construct or a subject interfering particle dissolved in diluents, such as water, saline, or fruit juice; capsules, sachets or tablets, each containing a predetermined amount of the active agent (a subject interfering construct or subject interfering particle), as solid or granules; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

Similarly, a formulation suitable for oral administration can include lozenge forms, that can comprise the active ingredient in a flavor, usually sucrose and acacia or tragacanth; pastilles comprising the active ingredient (a subject interfering construct or subject interfering particle) in an inert base, such as gelatin and glycerin, or sucrose and acacia; and mouthwashes comprising the active agent in a suitable liquid carrier; as well as creams, emulsions, gels, and the like containing, in addition to the active agent, such carriers as are available in the art.

A formulation for rectal administration can be presented as a suppository with a suitable base comprising, for example, cocoa butter or a salicylate. A formulation suitable for vaginal administration can be presented as a pessary, tampon, cream, gel, paste, foam, or spray formula containing, in addition to the active ingredient, such carriers as are known in the art to be appropriate. Similarly, the active ingredient can be combined with a lubricant as a coating on a condom.

The dose administered to an animal, particularly a human, in the context of the present invention should be sufficient to effect a therapeutic response in the infected individual over a reasonable time frame. The dose will be determined by the potency of the particular interfering construct or interfering particle employed for treatment, the severity of the disease state, as well as the body weight and age of the infected individual. The size of the dose also will be determined by the existence of any adverse side effects that can accompany the use of the particular interfering construct or interfering particle employed. It is always desirable, whenever possible, to keep adverse side effects to a minimum.

The dosage can be in unit dosage form, such as a tablet, a capsule, a unit volume of a liquid formulation, etc. The term “unit dosage form” as used herein refers to physically discrete units suitable as unitary dosages for human and animal subjects, each unit containing a predetermined quantity of an interfering construct or an interfering particle, alone or in combination with other antiviral agents, calculated in an amount sufficient to produce the desired effect in association with a pharmaceutically acceptable diluent, carrier, or vehicle. The specifications for the unit dosage forms of the present disclosure depend on the particular construct or particle employed and the effect to be achieved, as well as the pharmacodynamics associated with each construct or particle in the host. The dose administered can be an “antiviral effective amount” or an amount necessary to achieve an “effective level” in the individual patient.

Generally, an amount of a subject interfering construct or a subject interfering particle sufficient to achieve a tissue concentration of the administered construct or particle of from about 50 mg/kg to about 300 mg/kg of body weight per day can be administered, e.g., an amount of from about 100 mg/kg to about 200 mg/kg of body weight per day. In certain applications, e.g., topical, ocular or vaginal applications, multiple daily doses can be administered. Moreover, the number of doses will vary depending on the means of delivery and the particular interfering construct or interfering particle administered.

In some embodiments, a subject interfering construct or interfering particle (or composition comprising same) is administered in combination therapy with one or more additional therapeutic agents. Suitable additional therapeutic agents include agents that inhibit one or more functions of SARS-CoV-2 virus, agents that treat or ameliorate a symptom of SARS-CoV-2 virus infection; agents that treat an infection that may occur secondary to SARS-CoV-2 virus infection; and the like.

### Kits, Containers, Devices, Delivery Systems

Kits are described herein that include unit doses of the active agent (SARS-CoV-2 interfering particles or SARS-CoV-2 deletion nucleic acids). The unit doses can be formulated for nasal, oral, transdermal, or injectable (e.g., for intramuscular, intravenous, or subcutaneous injection) administration. In such kits, in addition to the containers containing the unit doses will be an informational package insert describing the use and attendant benefits of the drugs in treating SARS-CoV-2 infection. Suitable active agents (a subject interfering construct or a subject interfering particle) and unit doses are those described herein above.

In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In some embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

The present invention provides a delivery system comprising an active agent. In some embodiments, the delivery system is a delivery system that provides for injection of a formulation comprising an active agent subcutaneously, intravenously, or intramuscularly. In other embodiments, the delivery system is a vaginal or rectal delivery system.

In some embodiments, an active agent is packaged for oral administration. The present invention provides a packaging unit comprising daily dosage units of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the invention provides an injection delivery device that is pre-loaded with a formulation comprising an effective amount of a subject active agent. For example, a subject delivery device comprises an injection device pre-loaded with a single dose of a subject active agent. A subject injection device can be re-usable or disposable.

Pen injectors are available. Exemplary devices which can be adapted for use in the present methods are any of a variety of pen injectors from Becton Dickinson, e.g., BD™ Pen, BD™ Pen II, BD™ Auto-Injector: a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable.

In some embodiments, a subject delivery system comprises a device for delivery to nasal passages or lungs. For example, the compositions described herein can be formulated for delivery by a nebulizer, an inhaler device, or the like.

Bioadhesive microparticles constitute still another drug delivery system suitable for use in the context of the present disclosure. This system is a multi-phase liquid or semi-solid preparation that preferably does not seep from the nasal passages. The substances can cling to the nasal wall and release the drug over a period of time. Many of these systems were designed for nasal use (e.g. U.S. Pat. No. 4,756,907). The system may comprise microspheres with an active agent; and a surfactant for enhancing uptake of the drug. The microparticles have a diameter of 10-100 μm and can be prepared from starch, gelatin, albumin, collagen, or dextran.

Another system is a container comprising a subject formulation (e.g., a tube) that is adapted for use with an applicator. The active agent is incorporated into liquids, creams, lotions, foams, paste, ointments, and gels which can be applied to the vagina or rectum using an applicator. Processes for preparing pharmaceuticals in cream, lotion, foam, paste, ointment and gel formats can be found throughout the literature. An example of a suitable system is a standard fragrance-free lotion formulation containing glycerol, ceramides, mineral oil, petrolatum, parabens, fragrance and water such as the product sold under the trademark JERGENS™ (Andrew Jergens Co., Cincinnati, Ohio). Suitable nontoxic pharmaceutically acceptable systems for use in the compositions of the present invention will be apparent to those skilled in the art of pharmaceutical formulations and examples are described in Remington's Pharmaceutical Sciences, 19th Edition, A. R. Gennaro, ed., 1995. The choice of suitable carriers will depend on the exact nature of the particular vaginal or rectal dosage form desired, e.g., whether the active ingredient(s) is/are to be formulated into a cream, lotion, foam, ointment, paste, solution, or gel, as well as on the identity of the active ingredient(s). Other suitable delivery devices are those described in U.S. Pat. No. 6,476,079.

### Subjects to be Treated

The methods of the present disclosure are suitable for treating individuals who are suspected of having SARS-CoV-2 infection, and individuals who have SARS-CoV-2 infection, e.g., who have been diagnosed as having SARS-CoV-2 infection. The methods of the present disclosure are also suitable for use in individuals who have not been diagnosed as having SARS-CoV-2 infection (e.g., individuals who have been tested for SARS-CoV-2 and who have tested negative for SARS-CoV-2; and individuals who have not been tested), and who are considered at greater risk than the general population of contracting an SARS-CoV-2 infection (e.g., “at risk” individuals).

The methods of the present disclosure are suitable for treating individuals who are suspected of having SARS-CoV-2 infection, individuals who have SARS-CoV-2 infection (e.g., who have been diagnosed as having SARS-CoV-2 infection), and individuals who are considered at greater risk than the general population of contracting SARS-CoV-2 infection. Such individuals include, but are not limited to, individuals with healthy, intact immune systems, but who are at risk for becoming SARS-CoV-2 infected (“at-risk” individuals). In addition, such individuals include, but are not limited to, individuals that do not appear to have SARS-CoV-2 infection, but who may have reduced immune responses, heart disease, reduced lung capacity or a combination thereof (“at-risk” individuals). At-risk individuals include, but are not limited to, individuals who have a greater likelihood than the general population of becoming SARS-CoV-2 infection infected. Individuals at risk for becoming SARS-CoV-2 infected include, but are not limited to, essential services personnel such as medical personnel, emergency medical personnel, law enforcement, ambulance drivers, and public service drivers. Individuals at risk for becoming SARS-CoV-2 infected include, but are not limited to, older individuals (e.g., older than 65), immunocompromised individuals, individuals with heart disease, obese individuals, and individuals with other viral or bacterial infections. Individuals suitable for treatment therefore include individuals infected with, or at risk of becoming infected with SARS-CoV-2 or any variant thereof.

### Definitions

A “wild-type strain of a virus” is a strain that does not comprise any of the human-made mutations as described herein, i.e., a wild-type virus is any virus that can be isolated from nature (e.g., from a human infected with the virus). A wild-type virus can be cultured in a laboratory, but still, in the absence of any other virus, is capable of producing progeny genomes or virions like those isolated from nature.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of an agent (e.g., a construct, a particle, etc., as described herein) that, when administered to a mammal (e.g., a human) or other subject for treating a disease, is sufficient to effect such treatment for the disease. The “therapeutically effective amount” can vary depending on the compound or the cell, the disease and its severity and the age, weight, etc., of the subject to be treated.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

As used herein, a “pharmaceutical composition” is meant to encompass a composition suitable for administration to a subject, such as a mammal, e.g., a human. In general a “pharmaceutical composition” is sterile and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like.

All numerical designations, for example, temperature, time, concentration, viral load, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that in some cases equivalents may be available in the art.

Also, as used herein, “and/or” refers to and encompasses any and all possible combinations of one or more of the associated listed items, as well as the lack of combinations when interpreted in the alternative (“or”).

It must be noted that as used herein and in the appended claims, the singular forms “a,” “an,” and “the” include plural referents unless the context clearly dictates otherwise. Thus, for example, reference to “an interfering particle” includes a plurality of such particles and reference to “the cis-acting element” includes reference to one or more cis-acting elements and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The following examples illustrate some of the experimental work performed in the development of the invention.

### Example 1: Generating SARS-CoV-2 Random Deletion Libraries (RDLs)

To systematically identify regions of SARS-CoV-2 required for efficient mobilization, a randomized deletion screen was utilized similar to that described by Weinberger and Notton (2017), which created and index random-deletion libraries of HIV NL4-3.

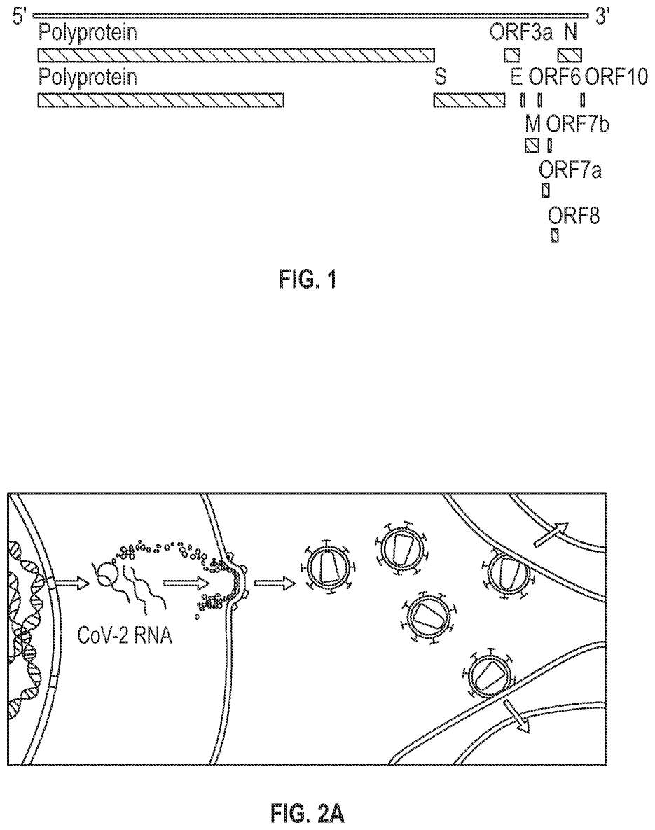

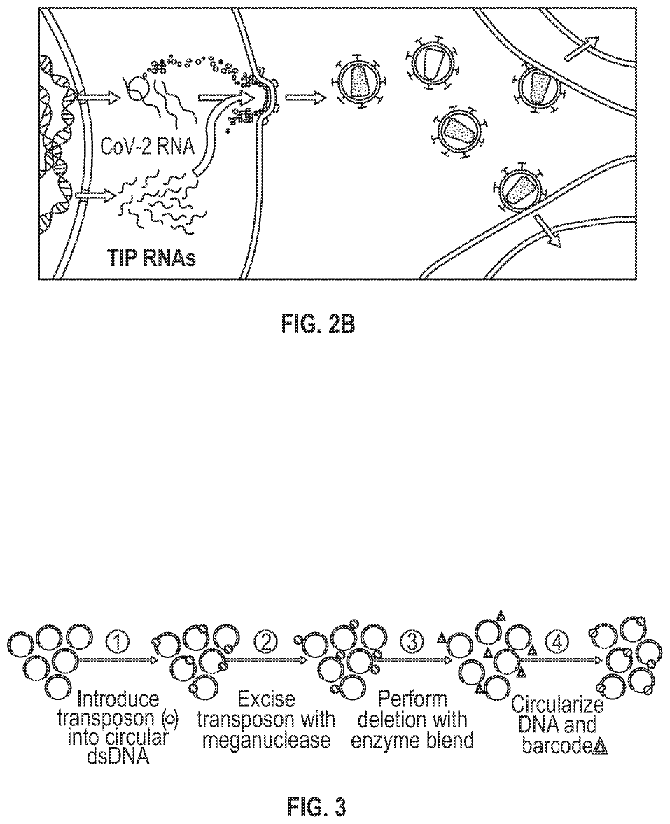

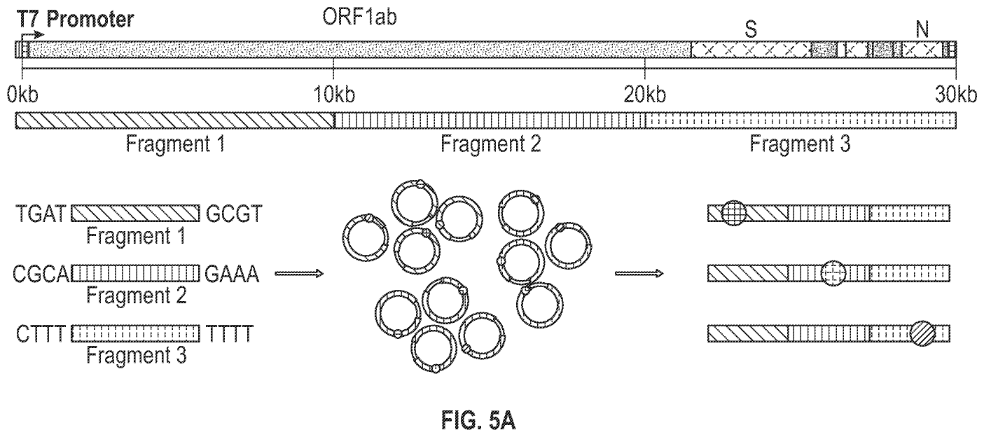

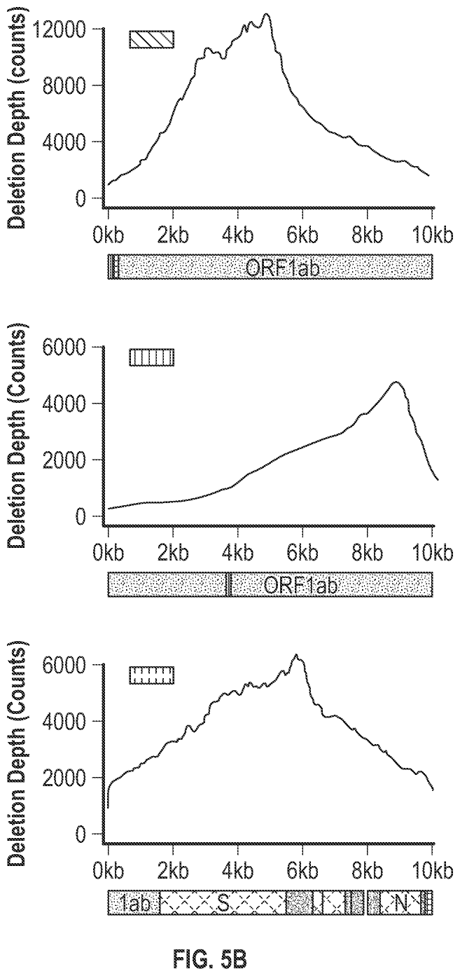

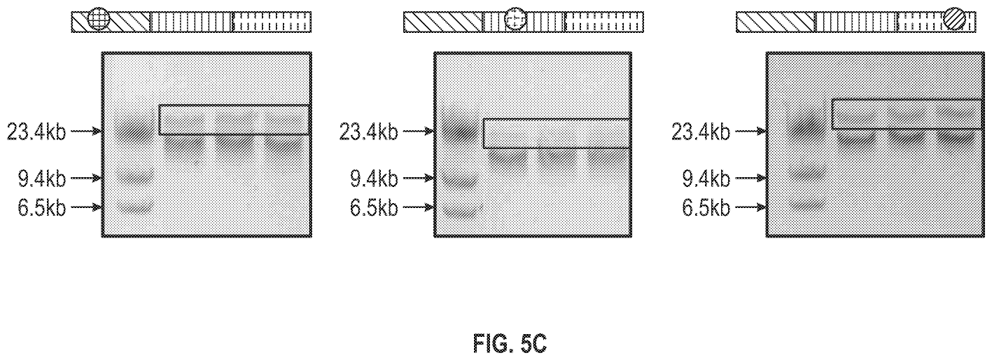

Briefly, plasmid DNA was subjected to transposon-mediated random insertion, followed by excision of the transposon and exonuclease-mediated digestion of the exposed ends to create deletions centered at a random genetic position, each of variable size. The plasmid was then re-ligated together with a cassette containing a 20-nucleotide random DNA barcode to ‘index’ the deletion. Indexing allows a deleted region to be easily identified (by the junction of genomic sequence and the barcode) and tracked/quantified by deep sequencing. This process is schematically illustrated in FIGS. 1-4. FIG. 5A further illustrates this process.

The deletion sites in the members of the libraries were sequenced. Deletion depth plots illustrated in FIG. 5B show that the sub-libraries contained over 587,000 deletions. The sub-libraries were ligated to form full-length libraries, the SARS-CoV-2 inserts were in vitro transcribed into RNA and the RNA was transfected into VeroE6 cells. The transfected cells were then infected with wild-type SARS-CoV-2 virus to test for mobilization of the deletion mutants. After three virus passages, RNA was extracted from cells and the presence of deletion barcodes was analyzed.

### SARS-CoV-2 Viroreactor

A SARS-CoV-2 viroreactor was set up using VeroE6 cells growing on silicone beads in suspension that can be infected with the SARS-CoV-2 deletion mutants, thereby creating a dynamic system to improve infection and ultimately evolution of SARS-CoV-2 therapeutic interfering particles (TIPs). The conditions used for the SARS-CoV-2 viroreactor were adapted from the protocol used to isolate an HIV TIP (described by Weinberger and Notton (2017)).

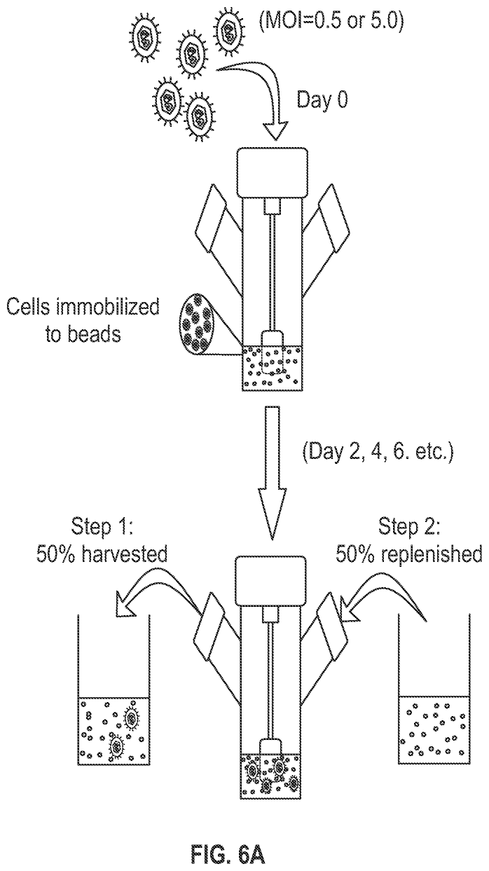

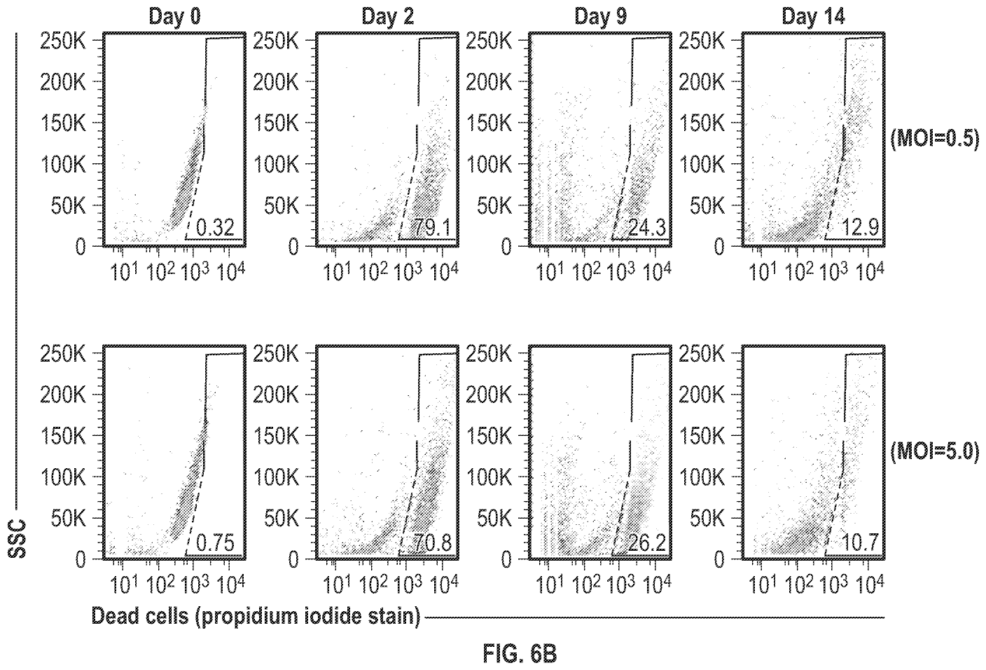

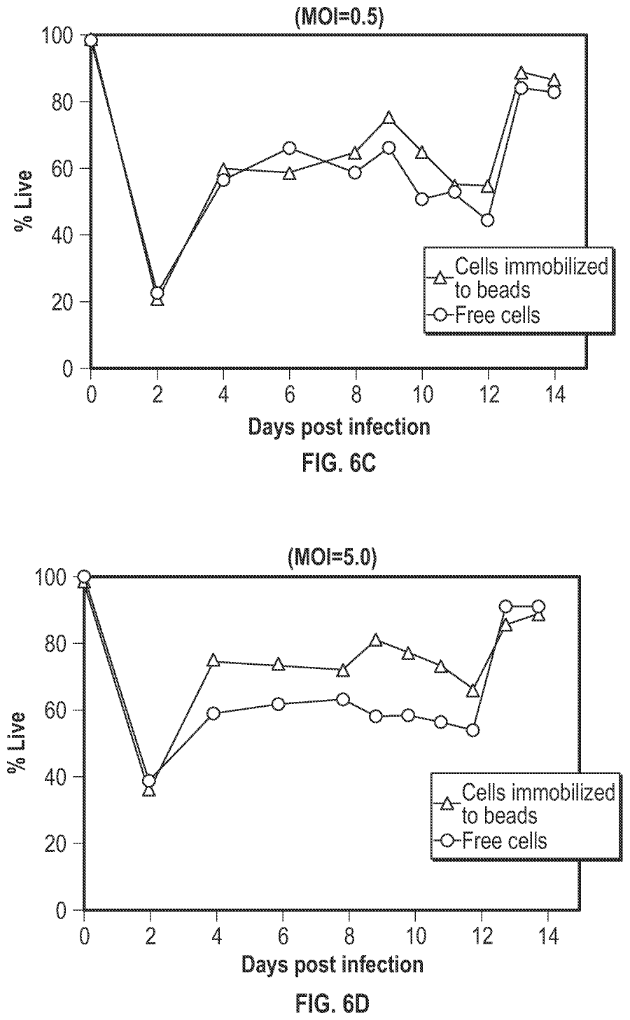

As illustrated in FIG. 6A, when the VeroE6 cells reached steady-state density, they were infected with the SARS-CoV-2 deletion mutants at a MOI of either 0.5 or 5, under gentle agitation. Half of the culture was removed from the reactor every day and replaced with fresh cells and media. Samples removed from the reactor were centrifugated, supernatants were frozen for later analysis and cell viability was measured by flow cytometry using a propidium iodine staining protocol (FIG. 6B). Cell viability was low (35-60%) at 2 days post infection (dpi) (FIG. 6C-6D) but started recovering as soon as 4 days post-infection (dpi) and stayed stable (60-805) until 12 dpi. At day 13, the cultures recovered to over 90% of cell viability.

### Example 2: SARS-CoV-2 Therapeutic Interfering Particles (TIPs)

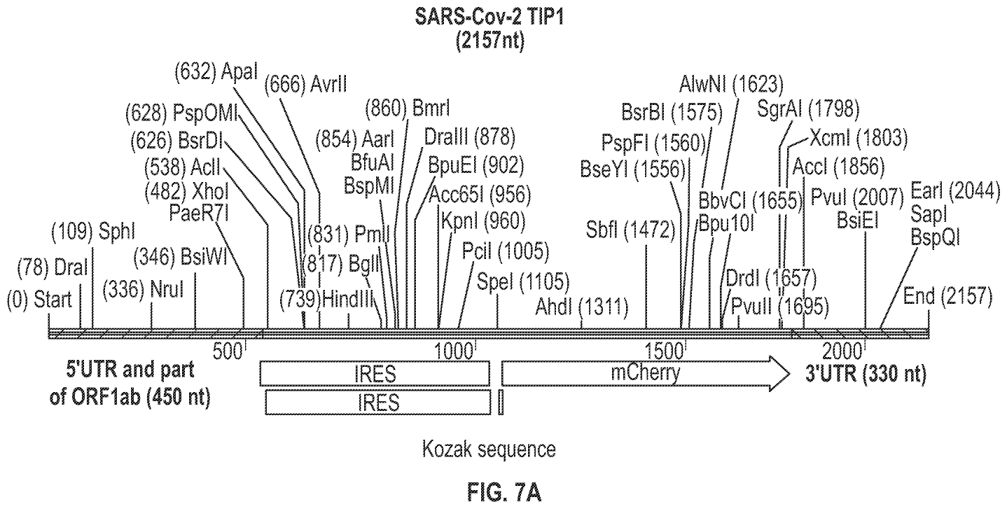

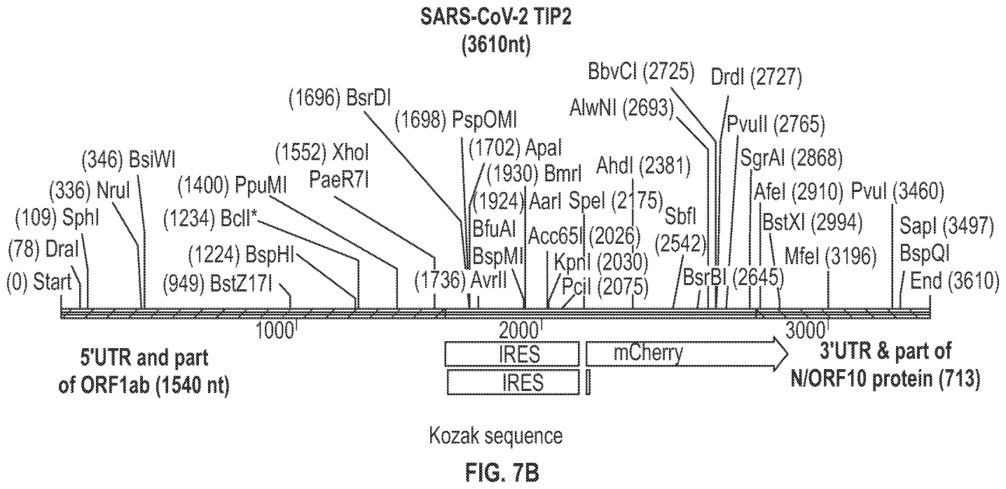

Minimal TIP constructs, TIP1 and TIP2, with the structures shown in FIG. 7A-7B were designed and cloned. The TIP1 and TIP2 constructs encode varying portions of the 5′ and 3′ UTRs of SARS-CoV-2 and express an mCherry reporter protein driven from an IRES. The plasmid constructs were sequence verified.

The 5′ SARS-CoV-2 sequences in TIP1 are as shown below (SEQ ID NO:28).

```
    1 ATTAAAGGTT TATACCTTCC CAGGTAACAA ACCAACCAAC
  41 TTTCGATCTC TTGTAGATCT GTTCTCTAAA CGAACTTTAA
  81 AATCTGTGTG GCTGTCACTC GGCTGCATGC TTAGTGCACT
121 CACGCAGTAT AATTAATAAC TAATTACTGT CGTTGACAGG
161 ACACGAGTAA CTCGTCTATC TTCTGCAGGC TGCTTACGGT
201 TTCGTCCGTG TTGCAGCCGA TCATCAGCAC ATCTAGGTTT
241 CGTCCGGGTG TGAGCGAAAG GTAAGATGGA GAGCCTTGTC
281 CCTGGTTTCA ACGAGAAAAC ACACGTCCAA CTCAGTTTGC
321 CTGTTTTACA GGTTCGCGAC GTGCTCGTAC GTGGCTTTGG
361 AGACTCCGTG GAGGAGGTCT TATCAGAGGC ACGTCAACAT
401 CTTAAAGATG GCACTTGTGG CTTAGTAGAA GTTGAAAAAG
411 GCGTTTTGCC
```

The 3′ SARS-CoV-2 sequences in TIP1 are shown below as SEQ ID NO:29.

```
    1 ATTAAAGGTT TATACCTTCC CAGGTAACAA ACCAACCAAC
  41 TTTCGATCTC TTGTAGATCT GTTCTCTAAA CGAACTTTAA
  81 AATCTGTGTG GCTGTCACTC GGCTGCATGC TTAGTGCACT
121 CACGCAGTAT AATTAATAAC TAATTACTGT CGTTGACAGG
161 ACACGAGTAA CTCGTCTATC TTCTGCAGGC TGCTTACGGT
201 TTCGTCCGTG TTGCAGCCGA TCATCAGCAC ATCTAGGTTT
241 CGTCCGGGTG TGACCGAAAG GTAAGATGGA GAGCCTTGTC
281 CCTGGTTTCA ACGAGAAAAC ACACGTCCAA CTCAGTTTGC
321 CTGTTTTACA GGTTCGCGAC GTGCTCGTAC GTGGCTTTGG
361 AGACTCCGTG GAGGAGGTCT TATCAGAGGC ACGTCAACAT
401 CTTAAAGATG GCACTTGTGG CTTAGTAGAA GTTGAAAAAG
411 GCGTTTTGCC
```

The 5′ SARS-CoV-2 sequences in TIP2 are as shown below (SEQ ID NO:30).

| | |
|---|---|
| 1 | ATTAAAGGTT TATACCTTCC CAGGTAACAA ACCAACCAAC |
| 41 | TTTCGATCTC TTGTAGATCT GTTCTCTAAA CGAACTTTAA |
| 81 | AATCTGTGTG GCTGTCACTC GGCTGCATGC TTAGTGCACT |
| 121 | CACGCAGTAT AATTAATAAC TAATTACTGT CGTTGACAGG |
| 101 | ACACGAGTAA CTCGTCTATC TTCTGCAGGC TGCTTACGGT |
| 201 | TTCGTCCGTG TTGCAGCCGA TCATCAGCAC ATCTAGGTTT |
| 241 | CGTCCGGGTG TGACCGAAAG GTAAGATGGA GAGCCTTGTC |
| 281 | CCTGGTTTCA ACGAGAAAAC ACACGTCCAA CTCAGTTTGC |
| 321 | CTGTTTTACA GGTTCGCGAC GTGCTCGTAC GTGGCTTTGG |
| 361 | AGACTCCGTG GAGGAGGTCT TATCAGAGGC ACGTCAACAT |
| 401 | CTTAAAGATG GCACTTGTGG CTTAGTAGAA GTTGAAAAAG |
| 441 | GCGTTTTGCC TCAACTTGAA CAGCCCTATG TGTTCATCAA |
| 481 | ACGTTCGGAT GCTCGAACTG CACCTCATGG TCATGTTATG |
| 521 | GTTGAGCTGG TAGCAGAACT CGAAGGCATT CAGTACGGTC |
| 561 | GTAGTGGTGA GACACTTGGT GTCCTTGTCC CTCATGTGGG |
| 601 | CGAAATACCA GTGGCTTACC GCAAGGTTCT TCTTCGTAAG |
| 641 | AACGGTAATA AAGGAGCTGG TGGCCATAGT TACGGCGCCG |
| 681 | ATCTAAAGTC ATTTGACTTA GGCGACGAGC TTGGCACTGA |
| 721 | TCCTTATGAA GATTTTCAAG AAAACTGGAA CACTAAACAT |
| 761 | AGCAGTGGTG TTACCCGTGA ACTCATGCGT GAGCTTAACG |
| 801 | GAGGGGCATA CACTCGCTAT GTCGATAACA ACTTCTGTGG |
| 841 | CCCTGATGGC TACCCTCTTG AGTGCATTAA AGACCTTCTA |
| 881 | GCACGTGCTG GTAAAGCTTC ATGCACTTTG TCCGAACAAC |
| 921 | TGGACTTTAT TGACACTAAG AGGGGTGTAT ACTGCTGCCG |
| 961 | TGAACATGAG CATGAAATTG CTTGGTACAC GGAACGTTCT |
| 1001 | GAAAAGAGCT ATGAATTGCA GACACCTTTT GAAATTAAAT |
| 1041 | TGGCAAAGAA ATTTGACACC TTCAATGGGG AATGTCCAAA |
| 1081 | TTTTGTATTT CCCTTAAATT CCATAATCAA GACTATTCAA |
| 1121 | CCAAGGGTTG AAAAGAAAAA GCTTGATGGC TTTATGGGTA |
| 1161 | GAATTCGATC TGTCTATCCA GTTGCGTCAC CAAATGAATG |
| 1201 | CAACCAAATG TGCCTTTCAA CTCTCATGAA GTGTGATCAT |
| 1241 | TGTGGTGAAA CTTCATGGCA GACGGGCGAT tttgttaaag |
| 1281 | CCACTTGCGA ATTTTGTGGC ACTGAGAATT TGACTAAAGA |
| 1321 | AGGTGCCACT ACTTGTGGTT AC1TAGCCCA AAATGCTGTT |
| 1361 | GTTAAAATTT ATTGTCCAGC ATGTCACAAT TCAGAAGTAG |
| 1401 | GACCTGAGCA TAGTCTTGCC GAATACCATA ATGAATCTGG |
| 1441 | CTTGAAAACC ATTCTTCGTA AGGGTGGTCG CACTATTGCC |
| 1481 | TTTGGAGGCT GTGTGTTCTC TTATGTTGGT TGCCATAACA |
| 1521 | AGTGTGCCTA TTGGGTTCCA gaattagatc tctcgaggtt |
| 1561 | aacgaattct gctatacgaa gttatccctc |

The 3′ SARS-CoV-2 sequences in TIP2 are as shown below (SEQ ID NO:31).

```
    1 ATTTGCCCCC AGCGCTTCAG CGTTCTTCGG AATGTCGCGC
  41 ATTGGCATGG AAGTCACACC TTCGGGAACG TGGTTGACCT
  81 ACACAGGTGC CATCAAATTG GATGACAAAG ATCCAAATTT
121 CAAAGATCAA GTCATTTTGC TGAATAAGCA TATTGACGCA
161 TACAAAACAT TCCCACCAAC AGAGCCTAAA AAGGACAAAA
201 AGAAGAAGGC TGATGAAACT CAAGCCTTAC CGUAGAGACA
241 GAAGAAACAG CAAACTGTGA CTCTTCTTCC TGCTGCAGAT
281 TTGGATGATT TCTCCAAACA ATTGCAACAA TCCATGAGCA
321 GTGCTGACTC AACTCAGGCC TAAACTCATG CAGACCACAC
361 AAGGCAGATG GGCTATATAA ACGTTTTCGC TTTTCCGTTT
401 ACGATATATA GTCTACTCTT GTGCAGAATG AATTCTCGTA
441 ACTAGATAGC ACAAGTAGAT GTAGTTAACT TTAATCTCAC
481 ATAGCAATCT TTAATCAGTG TGTAACATTA GGGAGGACTT
521 GAAAGAGCCA CCACATTTTC ACCGAGGCCA CGCGGAGTAC
561 GATCGAGTGT AGAGTGAACA ATGCTAGGGA GAGCTGCCTA
601 TATGGAAGAG CCCTAATGTG TAAAATTAAT TTTAGTAGTG
641 CTATCCCCAT GTGATTTTAA TAGCTTCTTA GGAGAATGAC
681 AAAAAAAAAA AAAAAAAAAA AAAAAAAAAA AAA
```

Two additional TIP variants were also cloned TIP11* and TIP2*, these contain the common C-241-T mutation within the 5′ UTR. This C241T UTR mutation co-transmits across populations together with the spike protein D614G mutation.

Hence, the 5′ SARS-CoV-2 sequences in TIP1* are as shown below (SEQ ID NO:32).

```
    1 ATTAAAGGTT TATACCTTCC CAGGTAACAA ACCAACCAAC
  41 TTTCGATCTC TTGTAGATCT GTTCTCTAAA CGAACTTTAA
  81 AATCTGTGTG GCTGTCACTC GGCTGCATGC TTAGTGCACT
121 CACGCAGTAT AATTAATAAC TAATTACTGT CGTTGACAGG
161 ACACGAGTAA CTCGTCTATC TTCTGCAGGC TGCTTACGGT
201 TTCGTCCGTG TTGCAGCCGA TCATCAGCAC ATCTAGGTTT
241 TGTCCGGGTG TGACCGAAAG GTAAGATGGA GAGCCTTGTC
281 CCTGGTTTCA ACGAGAAAAC ACACGTCCAA CTCAGTTTGC
321 CTGTTTTACA GGTTCGCGAC GTGCTCGTAC GTGGCTTTGG
361 AGACTCCGTG GAGGAGGTCT TATCAGAGGC ACGTCAACAT
401 CTTAAAGATG GCACTTGTGG CTTAGTAGAA GTTGAAAAAG
411 GCGTTTTGCC
```

Similarly, the SARS-CoV-2 sequences in TIP2* are as shown below (SEQ ID NO:33).

```
      1 ATTAAAGGTT TATACCTTCC CAGGTAACAA ACCAACCAAC
  201 TTCGTCCGTG TTGCAGCCGA TCATCAGCAC ATCTAGGTTT
  241 TGTCCGGGTG TGACCGAAAG GTAAGATGGA GAGCCTTGTC
  281 CCTGGTTTCA ACGAGAAAAC ACACGTCCAA CTCAGTTTGC
  321 CTGTTTTACA GGTTCGCGAC GTGCTCGTAC GTGGCTTTGG
  361 AGACTCCGTG GAGGAGGTCT TATCAGAGGC ACGTCAACAT
  401 CTTAAAGATG GCACTTGTGG CTTAGTAGAA GTTGAAAAAG
  441 GCGTTTTGCC 7CAACTTGAA CAGCCCTATG TGTTCATCAA
  481 ACGTTCGGAT GCTCGAACTG CACCTCATGG TCATGTTATG
  521 GTTGAGCTGG TAGCAGAACT CGAAGGCATT CAGTACGGTC
  561 GTAGTGGTGA GACACTTGGT GTCCTTGTCC CTCATGTGGG
  601 CGAAATACCA GTGGCTTACC GCAAGGTTCT TCTTCGTAAG
  641 AACGGTAATA AAGGAGCTGG TGGCCATAGT TACGGCGCCG
  681 ATCTAAAGTC ATTTGACTTA GGCGACGAGC TTGGCACTGA
  721 TCCTTATGAA GATTTTCAAG AAAACTGGAA CACTAAACAT
  761 AGCAGTGGTG TTACCCGTGA ACTCATGCGT GAGCTTAACG
  801 GAGGGGCATA CACTCGCTAT GTCGATAACA ACTTCTGTGG
  841 CCCTGATGGC TACCCTCTTG AGTGCATTAA AGACCTTCTA
  881 GCACGTGCTG GTAAAGCTTC ATGCACTTTG TCCGAACAAC
  921 TGGACTTTAT TGACACTAAG AGGGGTGTAT ACTGCTGCCG
  961 TGAACATGAG CATGAAATTG CTTGGTACAC GGAACGTTCT
1001 GAAAAGAGCT ATGAATTGCA GACACCTTTT GAAATTAAAT
1041 TGGCAAAGAA ATTTGACACC TTCAATGGGG AATGTCCAAA
1081 TTTTGTATTT CCCTTAAATT CCATAATCAA GACTATTCAA
1121 CCAAGGGTTG AAAAGAAAAA GCTTGATGGC TTTATGGGTA
1161 GAATTCGATC TGTCTATCCA GTTGCGTCAC CAAATGAATG
1201 CAACCAAATG TGCCTTTCAA ctctcatgaa GTGTGATCAT
1241 TGTGGTGAAA CTTCATGGCA GACGGGCGAT TTTGTTAAAG
1281 CCACTTGCGA ATTTTGTGGC ACTGAGAATT TGACTAAAGA
1321 AGGTGCCACT ACTTGTGGTT ACTTACCCCA AAATGCTGTT
1361 GTTAAAATTT ATTGTCCAGC ATGTCACAAT TCAGAAGTAG
1401 GACCTGAGCA TAGTCTTGCC GAATACCATA ATGAATCTGG
1441 CTTGAAAACC ATTCTTCGTA AGGGTGGTCG CACTATTGCC
1481 TTTGGAGGCT GTGTGTTCTC TTATGTTGGT TGCCATAACA
1521 AGTGTGCCTA TTGGGTTCCA gaattagatc tctcgaggtt
1561 aacgaattct gctatacgaa gttatccctc
```

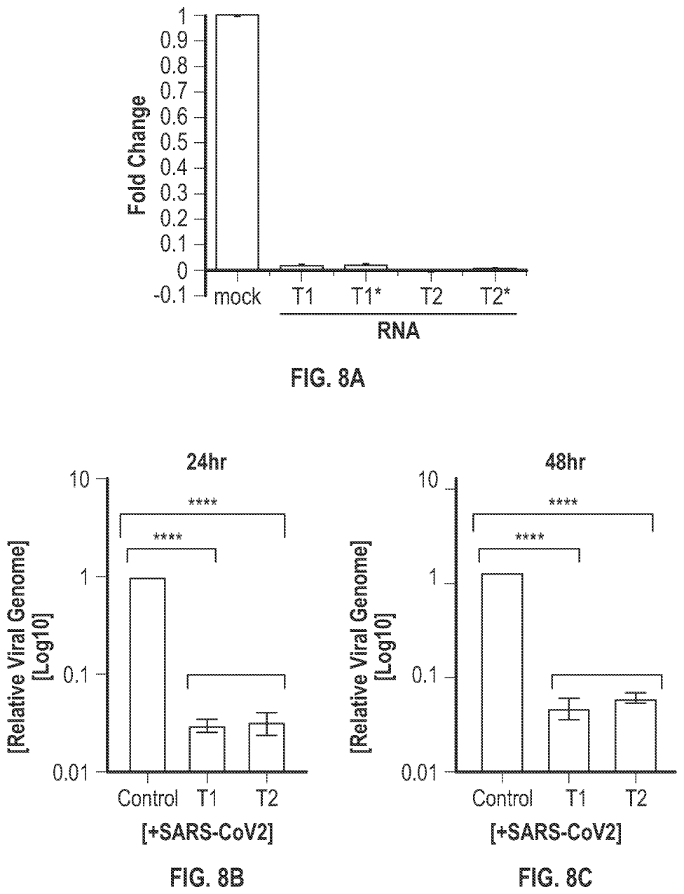

To test whether TIP constructs can reduce SARS-CoV-2 replication, mRNA from the four TIP constructs was generated by in vitro transcription from a T7 promoter operably linked upstream of the TIP in each plasmid. The different preparations of in vitro transcribed TIP mRNA were transfected into Vero E6 cells (TIP1, TIP1, TIP2, or TIP2*), and the cells were infected with SARS-CoV-2 (WA strain) at an MOI=0.005. At 48 hrs post-infection samples were harvested and a yield-reduction assay was conducted (see FIG. 8). Yield-reduction assays were measured by fold-reduction in SARS-CoV-2 mRNA (E gene) at 48 hrs post infection because the SARS-CoV-2 E (envelope) gene does not occur in the TIP sequences.

As shown in FIG. 8, all of the TIP constructs reduced SARS-CoV-2 viral replication but the TIP2 construct exhibited the greatest interference with SARS-CoV-2.

### Example 3: SARS-CoV-2 TIPs are Mobilized by SARS-CoV-2 and Transmit Together with SARS-CoV-2

Supernatant transfer experiments were performed to test the ability of the candidate TIPs to be mobilized by SARS-CoV-2 and transmitted together with SARS-CoV-2.

SARS-CoV-2-infected Vero E6 cells were transfected with various TIP candidates having the structures shown in FIGS. 7A-7B. Analysis for mCherry expression could therefore be used as a measure of TIP replication. Supernatant was collected from this first population of cells at 96 hours post-infection and the supernatant was transferred to a second population of fresh Vero cells. As a first control, supernatant was transferred from naïve uninfected cells to Vero cells, and as a second control supernatant was transferred from SARS-CoV-2 infected cells that were not transfected with TIPs. Flow cytometry was performed to analyze mCherry expression of the second population of cells at 48 hours after supernatant transfer.

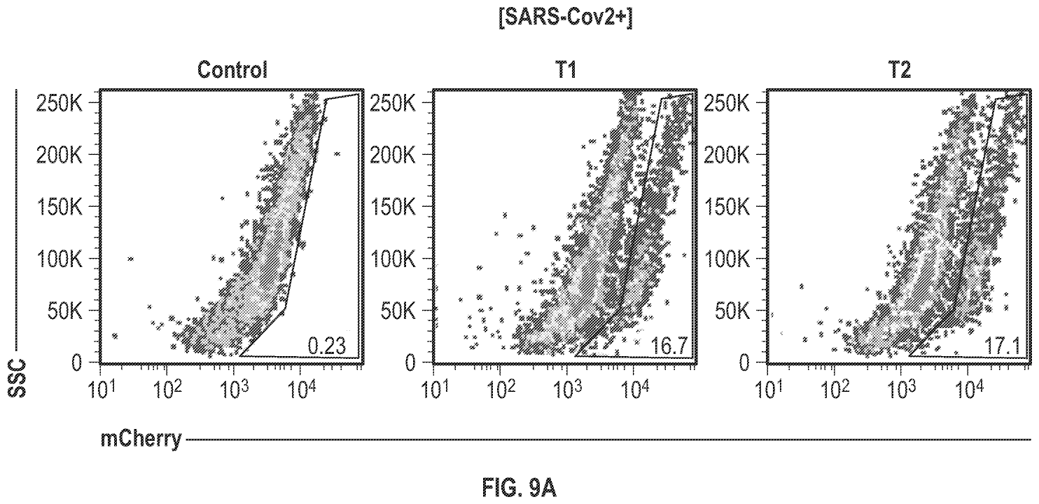

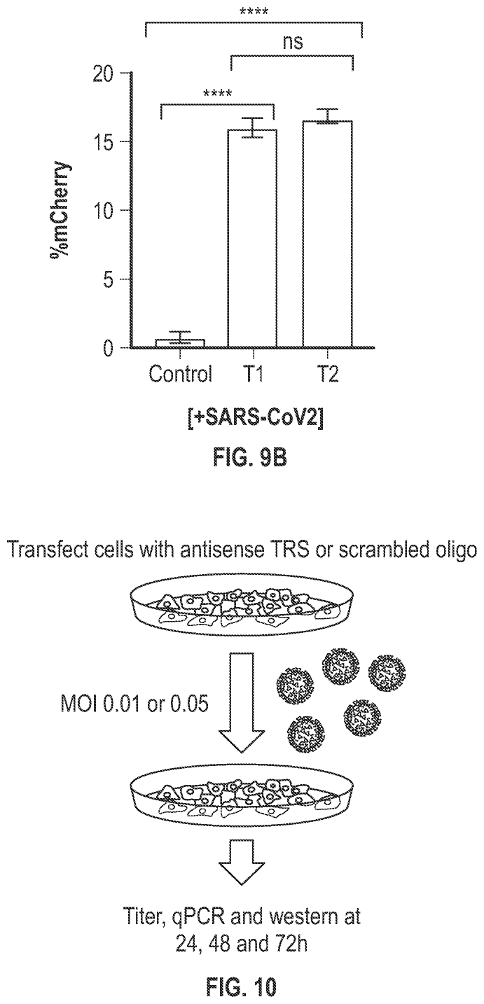

As shown in FIG. 9, the first and second controls showed no mCherry expression (FIG. 9A-9B). However, the supernatant from cells transfected with TIP candidate mRNA and infected with SARS-CoV-2 did generate mCherry producing cells, indicating that functional viral-like particles (VLPs) were being generated by SARS-CoV-2 helper virus (FIG. 9C-9I). In general, we found that mRNA transfection yielded better mobilization (FIG. 9G-9H) than DNA transfection (FIG. 9C-9F). This was consistent with results from the yield reduction assay by RT-qPCR where mRNA transfection also yielded better interference with SARS-CoV-2 than did DNA transfection (not shown).

### Example 4: Transcription Regulating Sequences (TRS) for Antiviral Intervention Against SARS-CoV-2

This Example describes use of antisense RNAs to intervene or interfere with SARS-CoV-2 infection.

Transcription initiation is regulated in coronaviruses by several types of consensus transcription regulating sequences (TRSs): TRS1-L: 5′-cuaaac-3′ (SEQ ID NO:36), TRS2-L: 5′-acgaac-3′ (SEQ ID NO:37), and TRS3-L, 5′-cuaaacgaac-3′ (SEQ ID NO:38).

To evaluate whether transcription can be inhibited from these transcriptional initiation sites, the following antisense TRS RNAs were developed:

TRS1-
(SEQ ID NO: 25)
ACGAACCUAAACACGAACCUAAAC;
TRS2-
(SEQ ID NO: 26)
(ACGAACACGAACACGAACACGAAC;
and
TRS3-
(SEQ ID NO :27)
CUAAACCUAAACCUAAACCUAAAC.

Vero cells were transfected with the antisense TRS RNAs and then infected with SARS-CoV-2 (MOI 0.01 or 0.05). As controls, cells were transfected with a scrambled RNA (instead of a TRS RNA) and then infected with SARS-CoV-2 (MOI 0.01 or 0.05). The titers of SARS-CoV-2 were determined by quantitative PCR and western blots were prepared at 24, 48, and 72 hours.

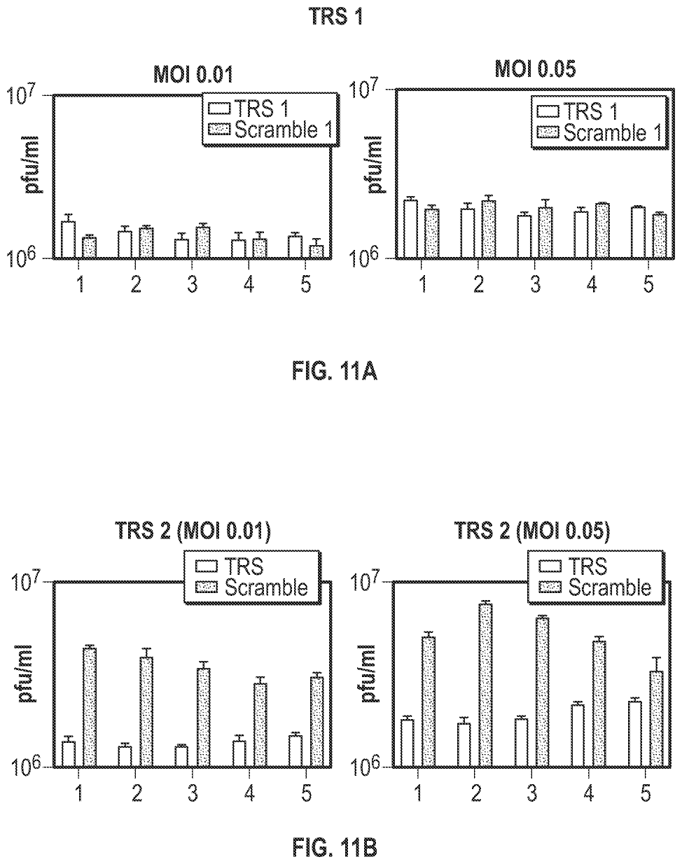

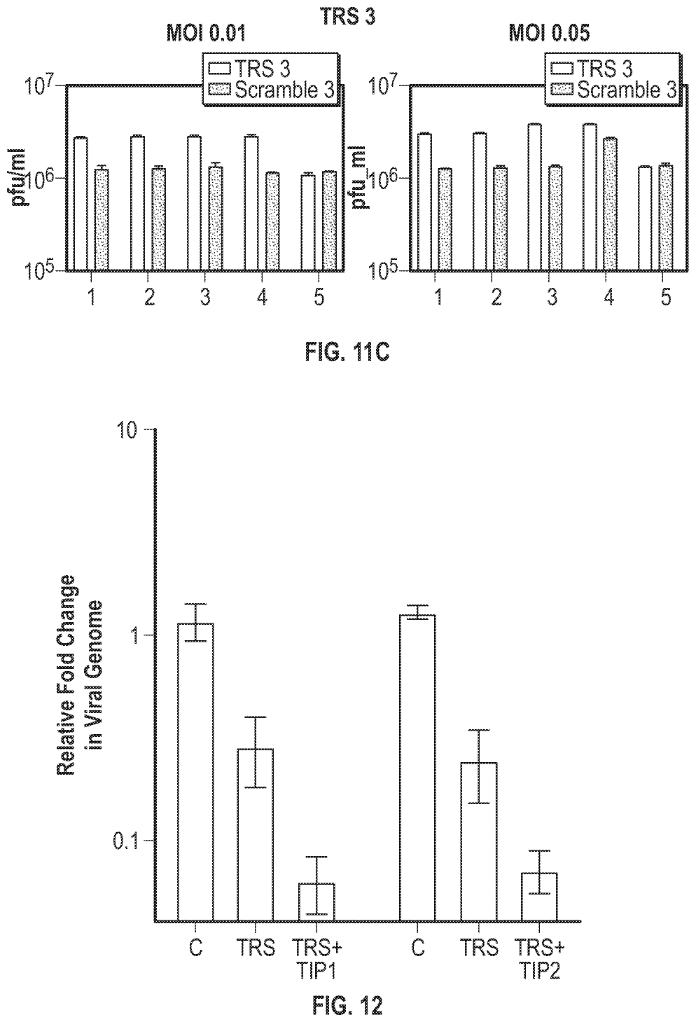

As shown in FIG. 11A-11C, use of the TRS2 antisense reduced SARS-CoV-2 titers to the greatest extent (FIG. 11B).

Vero cells were then incubated with combination of a TRS2 antisense with either TIP1 or TIP2, and then the cells were infected with SARS-CoV-2. The fold changes in SARS-CoV-2 genome numbers were then determined.

As shown in FIG. 12, the combination of the TRS2 antisense with either the TIP1 or TIP2 significantly reduced the SARS-CoV-2 genome numbers compared to the TRS alone.

### Example 5: SARS-CoV-2 TIPs Reduce Replication of Different SARS-CoV-2 Strains

This Example describes use of therapeutic interfering particles (TIP1 and TIP2) to intervene or interfere with different SARS-CoV-2 strains.

Vero cells were pretreated with lipid nanoparticles encapsulating therapeutic interfering particles (TIP1 or TIP2 at 0.3 ng/μl or 0.003 ng/μl), or a control RNA. At two hours post-treatment the cells were infected (MOI 0.005) with one of the following SARS-CoV-2 strains:
The 501Y.V2.HV variant of SARS-CoV-2, colloquially known as a South African variant;
The 501Y.V2.HV delta variant of SARS-CoV-2, colloquially known as a South African variant; and
The B.1.1.7 variant, colloquially known as a U.K. variant.
   Supernatant from the infected cultures was harvested at 48 hours post-infection and the SARS-CoV-2 viral titer was quantified.

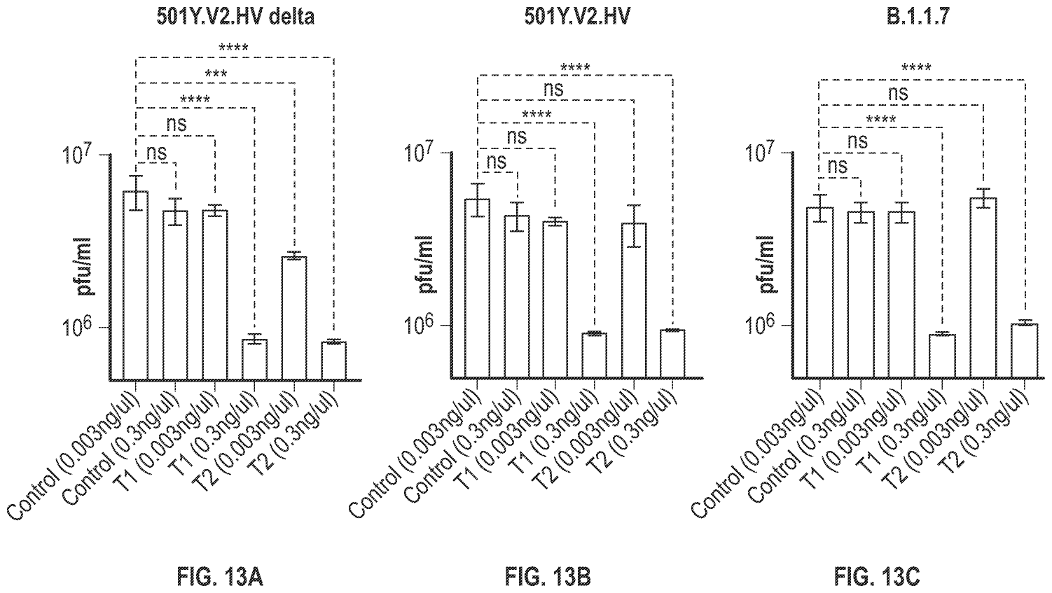

FIG. 13A-13C illustrate that TIP1 and TIP2 significantly reduce the replication of SARS-CoV-2 in a dose-dependent manner.

The following statements provide a summary of some aspects of the inventive nucleic acids and methods described herein.

### Statements:

1. A recombinant SARS-CoV-2 construct, the construct comprising: cis-acting elements comprising at least 100 nucleotides of a SARS-CoV-2 5′ untranslated region (5′ UTR), at least 100 nucleotides of a 3′ untranslated region (3′ UTR), or a combination thereof.
2. The construct of statement 1, which interferes with SARS-CoV-2 replication.
3. The construct of statement 1 or 2, which cannot replicate.
4. The construct of any one of statements 1-3, which replicates in the presence of infective SARS-CoV-2.
5. The construct of any one of statements 1-4, wherein the construct is incapable of replication and production of virus on its own but requires replication-competent SARS-CoV-2 to act as a helper virus.
6. The construct of any one of statements 1-5, which can be transmitted between cells in the presence of infective SARS-CoV-2.
7. The construct of any one of statements 1-6, comprising a packaging signal for SARS-CoV-2.
8. The construct of any one of statements 1-7, comprising deletion of portions of the SARS-CoV-2 genome encoding portions of any of SEQ ID NO:1-22.
9. The construct of statement 8, wherein the portions deleted from the genome comprise at least 10 to at least 27,000 nucleotides.
10. The construct of statement 8 or 9, wherein the portions deleted from the genome comprise at least 100, at least 500, at least 1000, at least 1500, at least 2000, at least 2500, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, at least 20,000, at least 21,000, at least 22,000, at least 23,000, at least 24,000, at least 25,000, at least 26,000, at least 27,000, at least 27500, or at least 28000 nucleotides of the SARS-CoV-2 genome.
11. The construct of any one of statements 1-10, wherein the SARS-CoV-2 construct blocks wild type SARS-CoV-2 cellular entry, competes for structural proteins that mediate viral particle assembly, exhibits reduced reproduction of the SARS-CoV-2 construct in vivo, produces proteins that inhibit assembly of viral particles, or a combination thereof.
12. The construct of statement 11, wherein SARS-CoV-2 genomic nucleic acids with one or more nucleotide sequence alterations compared to a wild type or native SARS-CoV-2 genomic nucleotide sequence;
13. The construct of statement 11 or 12, comprising one or more nucleotide sequence alterations in a spike protein membrane-fusing S2 subunit, an RNA-dependent RNA polymerase, a M protein (membrane glycoprotein), a ssRNA-binding protein, or a combination thereof in the SARS-CoV-2 construct genomic nucleic acids.
14. The construct of any of statements 1-13, wherein the SARS-CoV-2 construct genomic RNA is produced at a higher rate than wild-type SARS-CoV-2 genomic RNA when present in a host cell infected with a wild-type SARS-CoV-2, such that the ratio of the construct SARS-CoV-2 genomic RNA to the wild-type SARS-CoV-2 genomic RNA is greater than 1 in the cell.
15. The construct of any of statements 1-14, wherein the construct has a higher transmission frequency than the wild-type SARS-CoV-2.
16. The construct of any of statements 1-15, wherein the construct has a basic reproductive ratio (R0)>1.
17. The construct of any of statements 1-16, wherein the construct is packaged with the same or a higher efficiency than wild-type SARS-CoV-2 when present in a host cell infected with a wild-type SARS-CoV-2.
18. The construct of any of statements 1-17, wherein the construct comprises at least a 1-20 nucleotide deletion within positions 1-265, 266-805, 806-2719, 2720-8554, 8555-10054, 10055-10972, 10973-11842, 11843-12091, 12092-12685, 12686-13024, 13025-13441, 13442-13468, 13468-16236, 16237-18039, 18040-19620, 19621-20658, 20659-21552, 21563-25384, 266-13483, or a combination thereof, wherein the position numbers are relative to reference SARS-CoV-2 sequence SEQ ID NO:1.
19. The construct of any of statements 1-18, wherein the construct comprises at least a 1-20 nucleotide deletion within positions 21563-25384 of a spike glycoprotein coding region, within positions numbered 13442-16236 of an RNA-dependent RNA polymerase coding region, positions 26523-27191 of an M protein (membrane glycoprotein) coding region, positions 12686-13024 of a ssRNA-binding protein coding region, or a combination thereof, wherein the position numbers are relative to reference SARS-CoV-2 sequence SEQ ID NO:1.
20. The construct of any of statements 1-19, wherein the construct comprises deletion of at least 1, 2, 3, 4, 5, 6, 7, 8, 9, 10, 11, 12, 13, 14, 15, 16, 17, 18, 20, 25, 30, 35, 40, 45, 50, 55, 60, 65, 70, 75, 80, 85, 90, 95, 100, 110, 120, 130, 140, 150, 160 170, 180, 190, 200, 225, 250, 300, 350, 400, 450 or 500 nucleotides.
21. The construct of any of statements 1-20, wherein the construct comprises 5′ SARS-CoV-2 truncated sequences having any of SEQ ID NO:28, 30, 32 or 33.
22. The construct of any of statements 1-21, wherein the construct comprises 3′ SARS-CoV-2 truncated sequences such as any of those with SEQ ID NO:31 or 32.
23. The construct of any of statements 1-22 wherein the construct comprises extended poly A sequences.
24. The construct of statement 23, wherein the extended poly A sequences extend the half-life of the mRNA.
25. The construct of any of statements 23 or 24, wherein the extended poly A sequences, comprise at least 100 adenine nucleotides, at least 120 adenine nucleotides, at least 140 adenine nucleotides, at least 150 adenine nucleotides, at least 170 adenine nucleotides, at least 180 adenine nucleotides, at least 200 adenine nucleotides, at least 225 adenine nucleotides, or at least 250 adenine nucleotides.
26. The construct of any of statements 1-25, wherein the construct comprises a segment encoding a detectable marker.
27. A particle comprising the construct of any one of statements 1-26 and a viral envelope protein.
28. A pharmaceutical composition comprising the construct of any of statements 1-26 or the particle of statement 27 and a pharmaceutically acceptable excipient.
29. An inhibitor of SARS-CoV-2 transcription regulating sequences (TRSs) that can bind to one of more of: TRS1-L: 5′-cuaaac-3′ (SEQ ID NO:36), TRS2-L: 5′-acgaac-3′ (SEQ ID NO:37), TRS3-L, 5′-cuaaacgaac-3′ (SEQ ID NO:38), or a combination thereof.
30. The inhibitor of statement 29, comprising a sequence comprising or consisting essentially of:

TRS1-ACGAACCUAAACACGAACCUAAAC (SEQ ID NO:25);

TRS2-ACGAACACGAACACGAACACGAAC (SEQ ID NO:26);

TRS3-CUAAACCUAAACCUAAACCUAAAC (SEQ ID NO:27); or

a combination thereof.

31. A pharmaceutical composition comprising the inhibitor of statement 29 or 30 and a pharmaceutically acceptable excipient.
32. A pharmaceutical composition comprising a pharmaceutically acceptable excipient and the inhibitor of statement 29 or 30, the construct of any of statements 1-26, the particle of statement 27, or a combination thereof.
33. A method comprising administering the composition of statement 28, 31, or 32 to a subject.
34. The method of statement 33, wherein the subject is an experimental animal infected with SARS-CoV-2.
35. The method of statement 33, wherein the subject is a human.
36. The method of statement 33 or 35, wherein the subject is a human suspected of being infected with SARS-CoV-2, wherein the subject is a human who tested positive for SARS-CoV-2.
37. The method of any one of statements 33-36, wherein the subject has a medical condition, a pre-existing condition, or a condition that reduces heart, lung, brain or immune system function.
38. An isolated cell comprising the construct of any of statements 1-26 or the particle of statement 27.
39. A method of generating a deletion library, comprising:
(a) inserting a transposon cassette comprising a target sequence for a sequence specific DNA endonuclease into a population of circular SARS-CoV-2 DNAs to generate a population of transposon-inserted circular SARS-CoV-2 DNAs;
(b) contacting the population of transposon-inserted circular SARS-CoV-2 DNAs with the sequence specific DNA endonuclease to generate a population of cleaved linear SARS-CoV-2 DNAs;
(c) contacting the population of cleaved linear SARS-CoV-2 DNAs with one or more exonucleases to generate a population of deletion DNAs; and
(d) circularizing the deletion DNAs to generate a library of circularized SARS-CoV-2 deletion DNAs.
   40. The method of statement 39, wherein the circular SARS-CoV-2 DNAs are plasmids that comprise a SARS-CoV-2 genome.
   41. The method of statement 39 or 40, wherein the method further comprises introducing members of the library of circularized SARS-CoV-2 deletion DNAs into mammalian cells and assaying for SARS-CoV-2 viral infectivity.
   42. The method of one any of statements 39-41, wherein the method further comprises sequencing members of the library of circularized deletion DNAs to identify SARS-CoV-2 defective interfering particles (DIPs).
   43. The method of any one of statements 39-42, wherein the sequence specific DNA endonuclease is selected from: a meganuclease, a CRISPR/Cas endonuclease, a zinc finger nuclease, or a TALEN.
   44. The method of any one of statements 39-43 wherein the method comprises inserting a barcode sequence prior to or simultaneous with step (d).
   45. The method of any one of statements 39-44, wherein the one or more exonucleases comprises T4 DNA polymerase.
   46. The method of any one of statements 39-45, wherein the one or more exonucleases comprises a 3′ to 5′ exonuclease and a 5′ to 3′ exonuclease.
   47. The method of any one of statements 39-46, wherein the step of contacting the population of cleaved linear SARS-CoV-2 DNAs with one or more exonucleases is performed in the presence of a single strand binding protein (SSB).
   48. The method of any one of statements 39-47, wherein the transposon cassette comprises a first recognition sequence positioned at or near one end of the transposon cassette and a second recognition sequence positioned at or near the other end of the transposon cassette.
   49. The method of any one of statements 39-48, further comprising, prior to step (a), circularizing a population of SARS-CoV-2 linear DNA molecules to generate said population of circular SARS-CoV-2 DNAs.
   50. The method of statement 49, wherein the population of linear SARS-CoV-2 DNA molecules comprises one or more PCR products, one or more linear viral genomes, and/or one or more restriction digest products.
   51. The method of any one of statements 39-50, further comprising introducing members of the library of circularized SARS-CoV-2 deletion DNAs into mammalian cells.
   52. The method of any one of statements 39-51, further comprising generating from the library of circularized SARS-CoV-2 deletion DNAs, at least one of: linear double stranded DNA (dsDNA) products, linear single stranded DNA (ssDNA) products, linear single stranded RNA (ssRNA) products, and linear double stranded RNA (dsRNA) products.
   53. The method of statement 52, further comprising introducing said linear dsDNA products, linear ssDNA products, linear ssRNA products, and/or linear dsRNA products into mammalian cells.
   54. A method of generating and identifying a defective interfering particle (DIP), comprising:
(a) inserting a target sequence for a sequence specific DNA endonuclease into a population of circular SARS-CoV-2 viral DNAs, each SARS-CoV-2 viral DNA comprising a SARS-CoV-2 viral genome, to generate a population of sequence-inserted viral DNAs;
(b) contacting the population of sequence-inserted viral DNAs with the sequence specific DNA endonuclease to generate a population of cleaved linear viral DNAs;
(c) contacting the population of cleaved linear viral DNAs with an exonuclease to generate a population of deletion DNAs;
(d) circularizing the deletion DNAs to generate a library of circularized deletion viral DNAs; and
(e) sequencing members of the library of circularized deletion viral DNAs to identify deletion interfering particles (DIPs).
   55. The method of statement 54, comprising, prior to step (a), circularizing a population of linear DNA molecules to generate said population of circular SARS-CoV-2 viral DNAs.
   56. The method of statement 54 or 55, wherein the population of linear DNA molecules comprises one or more PCR products, one or more linear viral genomes, and/or one or more restriction digest products.
   57. The method of any one of statements 54-56, wherein the method comprises inserting a barcode sequence prior to or simultaneous with step (d).
   58. The method of any one of statements 54-57, further comprising (i) introducing members of the library of circularized SARS-CoV-2 deletion viral DNAs into mammalian cells; and (ii) assaying for viral infectivity.
   59. The method of any one of statements 54-58, further comprising: generating from the library of circularized SARS-CoV-2 deletion viral DNAs, at least one of: linear double stranded DNA (dsDNA) products, linear single stranded DNA (ssDNA) products, linear single stranded RNA (ssRNA) products, and linear double stranded RNA (dsRNA) products.
   60. The method of statement 59, further comprising:
introducing said linear dsDNA products, linear ssDNA products, linear ssRNA products, and/or linear dsRNA products into mammalian cells; and assaying for viral infectivity.
   61. The method of any of statements 39-60, wherein the inserting of step (a) comprises inserting a transposon cassette into the population of circular SARS-CoV-2 viral DNAs, wherein the transposon cassette comprises the target sequence for the sequence specific DNA endonuclease, and wherein said generated population of sequence-inserted viral DNAs is a population of transposon-inserted viral DNAs.
   62. The method of any one of statements 39-61, wherein the method comprises, after step (d), infecting mammalian cells in culture with members of the library of circularized deletion viral DNAs at a high multiplicity of infection (MOI), culturing the infected cells for a period of time ranging from 12 hours to 2 days, adding naive cells to the to the culture, and harvesting virus from the cells in culture.
   63. The method of any one of statements 39-62, wherein the method comprises, after step (d), infecting mammalian cells in culture with members of the library of circularized deletion viral DNAs at a low multiplicity of infection (MOI), culturing the infected cells in the presence of an inhibitor of viral replication for a period of time ranging from 1 day to 6 days, infecting the cultured cells with functional virus at a high MOI, culturing the infected cells for a period of time ranging from 12 hours to 4 days, and harvesting virus from the cultured cells.
   64. A method of treating an individual suspected of being infected with SARS-CoV-2 virus, the method comprising administering a therapeutically effective amount of the construct of any of statements 1-26, the particle of statement 27, or the pharmaceutical composition of statement 28 to the individual.
   65. The method of statement 64, further comprising administering a therapeutically effective amount of the composition of statement 31 or 32 to the individual.
   66. The method of statement 64 or 65, which reduces SARS-CoV-2 viral load in the individual.
   67. The method of any one of statements 64-66, wherein the individual has been diagnosed with SARS-CoV-2 infection or is considered to be at higher risk than the general population of becoming infected with SARS-CoV-2.
   68. A kit for treating an infection by SARS-CoV-2 virus comprising a container comprising of the construct of any of statements 1-26, the particle of statement 27, the pharmaceutical composition of statement 28, or the composition of statement 31 or 32 to the individual.
   69. The kit of statement 68, wherein the container is a syringe or a devise for administration to lungs or nasal passages.
   70. An isolated biological fluid comprising the construct of one of statements 1-26.
   71. The isolated biological fluid of statement 65, wherein the biological fluid is blood or plasma.
   72. A method of generating a particle, the method comprising transfecting a cell infected with SARS-CoV-2 virus with the construct of any of statements 1-26 and incubating the cell under conditions suitable for packaging the construct in the particle.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Under no circumstances may the patent be interpreted to be limited by any statement made by any Examiner or any other official or employee of the Patent and Trademark Office unless such statement is specifically and without qualification or reservation expressly adopted in a responsive writing by Applicants.

In addition, where the features or aspects of the invention are described in terms of Markush groups, those skilled in the art will recognize that the invention is also thereby described in terms of any individual member or subgroup members of the Markush group.

What is claimed:

1. A recombinant SARS-CoV-2 construct, the construct comprising: cis-acting elements comprising at least 100 nucleotides of a SARS-CoV-2 5′ untranslated region (5′ UTR), at least 100 nucleotides of a 3′ untranslated region (3′ UTR), or a combination thereof.

2. The recombinant SARS-CoV-2 construct of claim 1, which interferes with SARS-CoV-2 replication.

3. The recombinant SARS-CoV-2 construct of claim 1, which cannot replicate in cells.

4. The recombinant SARS-CoV-2 construct of claim 1, which replicates in the presence of infective SARS-CoV-2.

5. The recombinant SARS-CoV-2 construct of claim 1, which can be transmitted between cells in the presence of infective SARS-CoV-2.

6. The recombinant SARS-CoV-2 construct of claim 1, comprising a packaging signal for SARS-CoV-2.

7. The recombinant SARS-CoV-2 construct of claim 1, comprising deletion of portions of the SARS-CoV-2 genome encoding portions of any of SEQ ID NO:1-22.

8. The recombinant SARS-CoV-2 construct of claim 7, wherein the portions deleted from the genome comprise at least 10 to at least 27,000 nucleotides.

9. The recombinant SARS-CoV-2 construct of claim 1, wherein the SARS-CoV-2 construct blocks wild type SARS-CoV-2 cellular entry, competes for structural proteins that mediate viral particle assembly, exhibits reduced reproduction of the SARS-CoV-2 construct in vivo, produces proteins that inhibit assembly of viral particles, or a combination thereof.

10. The recombinant SARS-CoV-2 construct of claim 1, wherein the SARS-CoV-2 construct genomic RNA is produced at a higher rate than wild-type SARS-CoV-2 genomic RNA when present in a host cell infected with a wild-type SARS-CoV-2, such that the ratio of the construct SARS-CoV-2 genomic RNA to the wild-type SARS-CoV-2 genomic RNA is greater than one in the cell.

11. The recombinant SARS-CoV-2 construct of claim 1, wherein the construct has a higher transmission frequency than the wild-type SARS-CoV-2.

12. The recombinant SARS-CoV-2 construct of claim 1, wherein the construct has a basic reproductive ratio (R0)>1.

13. The recombinant SARS-CoV-2 construct of claim 1, wherein the construct is packaged with the same or a higher efficiency than wild-type SARS-CoV-2 when present in a host cell infected with a wild-type SARS-CoV-2.

14. The recombinant SARS-CoV-2 construct of claim 1, wherein the construct comprises 5′ SARS-CoV-2 truncated sequences having any of SEQ ID NO:28, 30, 32 or 33.

15. The recombinant SARS-CoV-2 construct of claim 14, wherein the construct comprises 3′ SARS-CoV-2 truncated sequences such as any of those with SEQ ID NO:31 or 32.

16. The recombinant SARS-CoV-2 construct of claim 1, wherein the construct comprises extended poly A sequences.

17. The recombinant SARS-CoV-2 construct of claim 16, wherein the extended poly A sequences comprise at least 100 adenine nucleotides.

18. The recombinant SARS-CoV-2 construct of claim 1, wherein the construct comprises a segment encoding a detectable marker.

19. A pharmaceutical composition comprising the recombinant SARS-CoV-2 construct of claim 1 and a pharmaceutically acceptable excipient.

20. An inhibitor of one or more SARS-CoV-2 transcription regulating sequences (TRSs) that can bind to one of more of: TRS1-L: 5′-cuaaac-3′ (SEQ ID NO:36), TRS2-L: 5′-acgaac-3′ (SEQ ID NO:37), TRS3-L, 5′-cuaaacgaac-3′ (SEQ ID NO:38), or a combination thereof.

21. The inhibitor of claim 20, comprising a sequence comprising or consisting essentially of:
TRS1-ACGAACCUAAACACGAACCUAAAC (SEQ ID NO:25);
TRS2-ACGAACACGAACACGAACACGAAC (SEQ ID NO:26);
TRS3-CUAAACCUAAACCUAAACCUAAAC (SEQ ID NO:27); or
a combination thereof.

22. A pharmaceutical composition comprising the inhibitor of claim 20 and a pharmaceutically acceptable excipient.

23. The pharmaceutical composition comprising a pharmaceutically acceptable excipient, (a) an inhibitor of SARS-CoV-2 transcription regulating sequences (TRSs) that can bind to one of more of: TRS1-L: 5′-cuaaac-3′ (SEQ ID NO:36), TRS2-L: 5′-acgaac-3′ (SEQ ID NO:37), TRS3-L, 5′-cuaaacgaac-3′ (SEQ ID NO:38), or a combination thereof; and (b) a recombinant SARS-CoV-2 construct, the construct comprising: cis-acting elements comprising at least 100 nucleotides of a SARS-CoV-2 5′ untranslated region (5′ UTR), at least 100 nucleotides of a 3′ untranslated region (3′ UTR), or a combination thereof.

24. A method for generating one or more defective interfering particles (DIPs), comprising:
(a) inserting a target sequence for a sequence specific DNA endonuclease into a population of circular SARS-CoV-2 viral DNAs, each SARS-CoV-2 viral DNA comprising a SARS-CoV-2 viral genome, or a portion of a SARS-CoV-2 viral genome, to generate a population of sequence-inserted viral DNAs;
(b) contacting the population of sequence-inserted viral DNAs with the sequence specific DNA endonuclease to generate a population of cleaved linear viral DNAs;
(c) contacting the population of cleaved linear viral DNAs with an exonuclease to generate a population of deletion DNAs;
(d) circularizing the deletion DNAs to generate a library of circularized deletion viral DNAs; and
(e) sequencing members of the library of circularized deletion viral DNAs to identify defective interfering particles (DIPs).

25. The method of claim 24, comprising, prior to step (a), circularizing a population of linear DNA molecules to generate said population of circular SARS-CoV-2 viral DNAs.

26. The method of claim 24, wherein the inserting step (a) comprises inserting a transposon cassette into the population of circular SARS-CoV-2 viral DNAs, wherein the transposon cassette comprises the target sequence for the sequence specific DNA endonuclease, and wherein said generated population of sequence-inserted viral DNAs is a population of transposon-inserted viral DNAs.

27. The method of claim 24, wherein the method comprises inserting a barcode sequence, an expression cassette encoding a marker, or a combination thereof, prior to or simultaneous with step (d).

28. The method of claim 24, further comprising introducing members of the library of circularized SARS-CoV-2 deletion viral DNAs, or one or more types of defective interfering particles (DIPs) into cultured mammalian cells; and assaying for SARS-CoV-2 viral infectivity.

29. The method of claim 24, further comprising:
transfecting mammalian cells with members of the library of circularized deletion viral DNAs, or with one or more types of defective interfering particles (DIPs);
infecting the mammalian cells with SARS-CoV-2 to generate an assay mixture;
culturing the assay mixture; and
assaying the assay mixture for SARS-CoV-2 viral infectivity, quantifying the circularized deletion viral DNAs or the defective interfering particles (DIPs), or a combination thereof.

30. The method of claim 24, further comprising:
transfecting mammalian cells with members of the library of circularized deletion viral DNAs, or with one or more types of defective interfering particles (DIPs);
infecting the mammalian cells with SARS-CoV-2 to generate an assay mixture;
culturing the assay mixture;
removing supernatant from the cultured mammalian cells;
adding the supernatant to a culture of naïve cells; and
quantifying the infective SARS-CoV-2, the circularized deletion viral DNAs, the defective interfering particles (DIPs), or a combination thereof.

31. A method of generating a particle, comprising transfecting a cell infected with SARS-CoV-2 virus with the construct of claim 1 and incubating the cell under conditions suitable for packaging the construct in the particle.

32. A method comprising administering to a subject a pharmaceutical composition comprising a pharmaceutically acceptable excipient and a therapeutically effective amount of at least one interfering, recombinant SARS-CoV-2 construct, the construct comprising cis-acting elements comprising a SARS-CoV-2 5′ untranslated region (5′ UTR), a SARS-CoV-2 3′ untranslated region (3′ UTR), or a combination thereof, or a particle comprising the interfering, recombinant SARS-CoV-2 construct.

33. The method of claim 32, further comprising administering to a subject an inhibitor of SARS-CoV-2 transcription regulating sequences (TRSs) that can bind to one of more of: TRS1-L: 5′-cuaaac-3′ (SEQ ID NO:36), TRS2-L: 5′-acgaac-3′ (SEQ ID NO:37), TRS3-L, 5′-cuaaacgaac-3′ (SEQ ID NO:38), a combination thereof, or a composition thereof.

34. The method of claim 32, further comprising measuring the SARS-CoV-2 viral load after 2-21 days.

35. The method of claim 32, wherein the subject is an individual or patient who tested positive for SARS-CoV-2 or wherein the subject is suspected of being infected with SARS-CoV-2.

36. The method of claim 32, wherein the subject is an individual or patient who is considered to be at higher risk than the general population of becoming infected with SARS-CoV-2 or has been diagnosed with SARS-CoV-2 infection.

37. Use of a pharmaceutical composition comprising:
a therapeutically effective amount of at least one interfering, recombinant SARS-CoV-2 construct, the construct comprising cis-acting elements comprising a SARS-CoV-2 5′ untranslated region (5′ UTR), a SARS-CoV-2 3′ untranslated region (3′ UTR), or a combination thereof, or a particle comprising the interfering, recombinant SARS-CoV-2 construct, and a pharmaceutically acceptable excipient, in the treatment or prevention of SARS-CoV-2 infection;
a therapeutically effective amount of at least one inhibitor of SARS-CoV-2 transcription regulating sequences (TRSs), wherein the inhibitor can bind to one of more of: TRS1-L: 5′-cuaaac-3′ (SEQ ID NO:36), TRS2-L: 5′-acgaac-3′ (SEQ ID NO:37), TRS3-L, 5′-cuaaacgaac-3′ (SEQ ID NO:38);
or a combination thereof,
in the treatment or inhibition of SARS-CoV-2 infection.

38. A kit for treating an infection by SARS-CoV-2 virus comprising:
a container comprising a therapeutically effective amount of at least one recombinant SARS-CoV-2 construct, the construct comprising cis-acting elements comprising a SARS-CoV-2 5′ untranslated region (5′ UTR), a SARS-CoV-2 3′ untranslated region (3′ UTR), a combination thereof, or a pharmaceutical composition thereof;
a container comprising a composition comprising particles comprising the recombinant SARS-CoV-2 construct;
a container comprising at least one inhibitor of SARS-CoV-2 transcription regulating sequences (TRSs) that can bind to one of more of: TRS1-L: 5′-cuaaac-3′ (SEQ ID NO:36), TRS2-L: 5′-acgaac-3′ (SEQ ID NO:37), TRS3-L, 5′-cuaaacgaac-3′ (SEQ ID NO:38), or a combination thereof;
a container comprising a composition comprising the at least one inhibitor of SARS-CoV-2 transcription regulating sequences; and
instructions for using the recombinant SARS-CoV-2 construct, the at least one inhibitor of SARS-CoV-2 transcription regulating sequences, and the composition(s) thereof.

39. The kit of claim 38, wherein the container is a syringe or a devise for administration to lungs or nasal passages.
